(19) United States
(12) Patent Application Publication
(10) Pub. No.: US 2017/0190781 A1
(43) Pub. Date: Jul. 6, 2017

(54) ANTI-DEATH RECEPTOR 3 (DR3) ANTAGONISTIC ANTIBODIES WITH REDUCED AGONISTIC ACTIVITY

(71) Applicant: KYOWA HAKKO KIRIN CO., LTD, Tokyo (JP)

(72) Inventors: David MILLS, Seattle, WA (US); Kazuma TOMIZUKA, Tokyo (JP); John LAUDENSLAGER, La Jolla, CA (US); Rinpei NIWA, Tokyo (JP); Mami TSUCHIYA, Tokyo (JP)

(73) Assignee: KYOWA HAKKO KIRIN CO., LTD, Tokyo (JP)

(21) Appl. No.: 15/301,676

(22) PCT Filed: Oct. 8, 2015
(86) PCT No.: PCT/JP2015/061298

§ 371 Date: Oct. 3, 2016

Related U.S. Application Data
(60) Provisional application No. 61/975,214, filed on Apr. 4, 2014.

(51) Int. Cl. C07K 16/28

(52) U.S. Cl. CPC C07K 16/2875; C07K 2317/56; C07K 2317/565; C07K 2317/35; C07K 2319/30

(57) ABSTRACT

According to the present invention, anti-death receptor 3 (DR3) antagonistic IgG antibodies and antibody fragments thereof, wherein the antibodies and the antibody fragments thereof display a decreased agonistic activity or no agonistic activity for DR3 through their binding, an antibody compositions and an antibody fragment compositions comprising them, a nucleotide sequence encoding the antibody or the antibody fragment, a vector comprising the nucleotide sequences, an amino acid sequences of the antibodies or the antibody fragments, a method of producing the antibodies or the antibody fragments thereof, and a method of decreasing the agonistic potency of an antibody against DR3 through its binding, are provided.

### TECHNICAL FIELD

The present invention relates to an anti-death receptor 3 (DR3) antagonistic antibodies with reduced agonistic activity.

### BACKGROUND ART

Death receptor 3 (DR3) is a member of a tumor necrosis factor receptor super-family (TNFRSF) and it has known as a tumor necrosis factor receptor super-family 25 (TNFRSF25), lymphocyte-associated receptor of death (LARD), APO-3, TRAMP and WSL-1. DR3 is expressed on mainly T cells and some other cells including endothelial cells, epithelial cells, osteoblasts, B cells, natural killer T (NKT) cells and type 2 innate lymphoid cells (ILC2) (NPL 1). DR3 activation is mediated by a TNF-like ligand, TL1A (TNF super family 15; TNFSF15) which is expressed in endothelial cells as well as lymphocyte, plasma cells, dendritic cells, macrophages, and monocytes (NPL 2). TL1A ligand binding to DR3 elicits proliferation, activation of T cells, and increases the secretion of inflammatory cytokines as interferon-γ (IFN-γ), interleukin (IL)-2, IL-13, tumor necrosis factor alpha (TNF-α), and granulocyte-macrophage colony stimulating factor (GM-CSF) from T cells. DR3 has also been shown to regulate in vivo NKT cell (NPL 3), and ILC2 cytokine production, most notably IL-13. Accordingly, transgene-mediated TL1A overexpression promotes IL-13-dependent intestinal pathology in mice (NPL 4).

Engaging of TL1A and DR3 has been related to several inflammatory diseases as inflammatory diseases such as inflammatory bowel diseases (IBD), ulcerative colitis, Crohn's disease, rheumatoid arthritis, asthma, and multiple sclerosis.

According to anti-DR3 antibodies, Wen et al (NPL 5) disclose anti-DR3 mouse monoclonal antibody F05 which has an agonistic activity for DR3, Novus Biologicals is supplying anti-DR3 mouse monoclonal antibody 1H2, being applicable for ELISA (NPL 6), Yu et al (PTL 1) and Tittle et al (PTL 2) disclose anti-TR3 antibody which inhibits T cell proliferation. On the other hand, Migone et al (PTLs 3, 4) disclose that mouse monoclonal antibody 11H08 binds DR3 and activates the DR3 receptor, anti-DR3 Fab fragment of 11H08 is generated, in order to obtain a monomeric anti-DR3 fragments that can inhibit the activity of TL1A through the DR3 receptor. Further Andersen et al (PTL 5) disclose that antagonistic DR3 ligands, such as a monovalent Fab fragments for DR3 that block binding of TL1A to DR3.

### CITATION LIST

### Patent Literature

PTL 1: U.S. Pat. No. 7,357,927
PTL 2: U.S. Pat. No. 6,994,976
PTL 3: WO2011/106707
PTL 4: US2012/0014950
PTL 5: WO2012/117067

### Non Patent Literature

NPL 1: Meylan et al, Mucosal Immunol., 2013; doi: 10.1038/mi.2013.1141-11
NPL 2: Fang et al, J Exp Med., 2008; 205:1037-1048
NPL 3: Migone at al, Immunity, 2002; 16: 479-492
NPL 4: Meylan et al, Mucosal Immunol., 2010; 4; 172-185
NPL 5: Wen et al, The Journal of Biological Chemistry, 2003; 278: 39251-39258
NPL 6: Data sheet of 1H2 mouse monoclonal antibody, cat. no. H00008718-M08

### SUMMARY OF INVENTION

### Technical Problem

DR3 antagonism can reduce inflammatory responses. However, previously known bivalent anti-DR3 antagonistic antibodies are all reported to have agonistic activity, which may promote deleterious inflammatory responses such as T cell proliferation and cytokine production by T cells. Therefore, all previously known DR3 antagonistic antibodies have been proposed for anti-inflammatory use only in monovalent formats of Fab.

### Solution to Problem

Because IgG antibodies and antibody fragments thereof generally display predictable composition and are easily purified using standardized methodology, the development and production of IgG formats or antibody fragment comprising a Fc region for therapeutic use is reasonably straightforward, affording some advantage over known monovalent formats. We have discovered potently antagonistic IgG antibody and antagonistic antibody fragment thereof for DR3 that decrease agonistic activity/do not display significant agonistic activity.

The present invention relates to the following (1) to (14).

(1) An immunoglobulin G (hereinafter described as IgG) antibody which binds to death receptor 3 (DR3) and antagonizes TL1A induced DR3 activation, wherein the antibody has a decreased or no agonistic activity against DR3 through their binding, or an antibody fragment thereof.

(2) The antibody or the antibody fragment thereof described in the above item (1), which binds to an epitope presented in a cysteine-rich domain (hereinafter described as CRD) of DR3.

(3) The antibody or the antibody fragment thereof described in the above item (1), which binds to an epitope comprising at least one amino acid residue presented in CRD1 or CRD4 of DR3.

(4) The antibody or the antibody fragment thereof described in the above item (1), which is one selected from an IgG2 antibody, an IgG2 antibody variant comprising a hinge domain of IgG2, and a domain exchanged antibody between IgG2 and IgG4, wherein an amino acid residue is Lys at EU numbering position 409.

(5) The antibody or the antibody fragment thereof described in the above item (1), which neutralizes and/or antagonizes an activity of DR3 induced through TL1A ligand binding.

(6) The antibody or the antibody fragment thereof described in any one of the above items (1) to (5), wherein the agonistic activity is at least one selected from the phosphorylation of p65 subunit of NF-kappa B, cytokine release from DR3 expressed cells, the proliferation of DR3 expressed cells, the apoptosis of DR3 expressed cells.

(7) The antibody or the antibody fragment thereof described in any one of the above items (1) to (6), which is at least one antibody selected from (i) to (iii) as described following;
(i) an antibody which competitively binds to DR3 with the anti-DR3 monoclonal antibody 142A2 or 142S38B,
(ii) an antibody which binds to an epitope presented in the epitope recognized by the anti-DR3 monoclonal antibody 142A2 or 142S38B, and
(iii) an antibody which binds to same epitope recognized by the anti-DR3 monoclonal antibody 142A2 or 142S38B.

(8) The antibody or the antibody fragment thereof described in the above item (1), which comprises an amino acid sequence of VH of SEQ ID NO:15 and an amino acid sequence of VL of SEQ ID NO:21, an amino acid sequence of VH of SEQ ID NO:76 and an amino acid sequence of VL of SEQ ID NO:21, an amino acid sequence of VH of SEQ ID NO:15 and an amino acid sequence of VL of SEQ ID NO:77, an amino acid sequence of VH of SEQ ID NO:15 and an amino acid sequence of VL of SEQ ID NO:78, an amino acid sequence of VH of SEQ ID NO:15 and an amino acid sequence of VL of SEQ ID NO:79, an amino acid sequence of VH of SEQ ID NO:76 and an amino acid sequence of VL of SEQ ID NO:79, or an amino acid sequence of VH of SEQ ID NO:27 and an amino acid sequence of VL of SEQ ID NO:33.

(9) The antibody or the antibody fragment thereof described in the above item (1), which comprises an amino acid sequences of CDR1 to CDR3 of VH of SEQ ID NOs:16 to 18, respectively, and an amino acid sequences of CDR1 to CDR3 of VL of SEQ ID NOs:22 to 24, respectively; an amino acid sequences of CDR1 to CDR3 of VH of SEQ ID NOs:16, 17, 80, respectively, and an amino acid sequences of CDR1 to CDR3 of VL of SEQ ID NOs:22-24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 81, 24, respectively; an amino acid sequences of CDR1 to CDR3 of VH of SEQ ID NOs: 16, 17, 80, respectively, and an amino acid sequences of CDR1 to CDR3 of VL of SEQ ID NOs:22, 82, 24, respectively; an amino acid sequences of CDR1 to CDR3 of VH of SEQ ID NOs:16-18, respectively, and an amino acid sequences of CDR1 to CDR3 of VL of SEQ ID NOs:22, 81, 24, respectively; an amino acid sequences of CDR1 to CDR3 of VH of SEQ ID NOs: 16-18, respectively, and an amino acid sequences of CDR1 to CDR3 of VL of SEQ ID NOs:22, 82, 24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 83, 24, respectively; or an amino acid sequences of CDR1 to CDR3 of VH of SEQ ID NOs:28 to 30, respectively, and amino acid sequences of CDR1 to CDR3 of VL of SEQ ID NOs:34 to 36, respectively.

(10) The antibody or the antibody fragment described in the above item (1), wherein the antibody fragment is selected from Fab, Fab′, F(ab′)₂, single chain Fv (scFv), diabody, disulfide stabilized Fv (dsFv), a peptide comprising six CDRs of the antibody and a Fc fusion proteins.

(11) The antibody or the antibody fragment thereof described in the above item (10), wherein the Fe fusion protein is an Fab or scFv fused to a Fc region selected from as following;
(i) a bivalent antibody in that two Fabs or scFvs are fused to Fc region of IgG class,
(ii) a monovalent antibody in that one Fab or scFv is fused to Fc region, and
(iii) a monovalent antibody comprising a H chain and a Fc-fused L-chain (hereinafter described as FL).

(12) The antibody or the antibody fragment thereof described in the above item (11), wherein the Fc region is selected from IgG1, IgG2, IgG4 and a variant thereof.

(13) The antibody or the antibody fragment hereof described in the above item (10), which comprises an amino acid sequence of VH of SEQ ID NO:15 and an amino acid sequence of VL of SEQ ID NO:21, an amino acid sequence of VH of SEQ ID NO:76 and an amino acid sequence of VL of SEQ ID NO:21, an amino acid sequence of VH of SEQ ID NO:15 and an amino acid sequence of VL of SEQ ID NO:77, an amino acid sequence of VH of SEQ ID NO:15 and an amino acid sequence of VL of SEQ ID NO:78, an amino acid sequence of VH of SEQ ID NO:15 and an amino acid sequence of VL of SEQ ID NO:79, an amino acid sequence of VH of SEQ ID NO:76 and an amino acid sequence of VL of SEQ ID NO:79, or an amino acid sequence of VH of SEQ ID NO:27 and an amino acid sequence of VL of SEQ ID NO:33.

(14) The antibody or the antibody fragment hereof described in the above item (10), which comprises an amino acid sequences of CDR1 to CDR3 of VH of SEQ ID NOs:16 to 18, respectively, and amino acid sequences of CDR1 to CDR3 of VL of SEQ ID NOs:22 to 24, respectively; an amino acid sequences of CDR1 to CDR3 of VH of SEQ ID NOs:16, 17, 80, respectively, and an amino acid sequences of CDR1 to CDR3 of VL of SEQ ID NOs:22-24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 81, 24, respectively; an amino acid sequences of CDR1 to CDR3 of VH of SEQ ID NOs: 16, 17, 80, respectively, and an amino acid sequences of CDR1 to CDR3 of VL of SEQ ID NOs:22, 82, 24, respectively; an amino acid sequences of CDR1 to CDR3 of VH of SEQ ID NOs:16-18, respectively, and an amino acid sequences of CDR1 to CDR3 of VL of SEQ ID NOs:22, 81, 24, respectively; an amino acid sequences of CDR1 to CDR3 of VH of SEQ ID NOs: 16-18, respectively, and an amino acid sequences of CDR1 to CDR3 of VL of SEQ ID NOs:22, 82, 24, respectively; an amino acid sequences of CDR to CDR3 of VH of SEQ ID NOs:16, 17, 80, respectively, and an amino acid sequences of CDR1 to CDR3 of VL of SEQ ID NOs:22, 83, 24, respectively; or amino acid sequences of CDR1 to CDR3 of VH of SEQ ID NOs:28 to 30, respectively, and amino acid sequences of CDR1 to CDR3 of VL of SEQ ID NOs:34 to 36, respectively.

### Advantageous Effects of Invention

Since the antibody and antibody fragment of the present invention exhibit decreased or no agonistic activity, they can be used for treating, preventing or ameliorating inflammatory diseases, autoimmune diseases, cancer diseases and symptoms associated with their diseases.

### BRIEF DESCRIPTION OF DRAWINGS

FIG. 1A and FIG. 1B indicate typical Biacore sensorgrams of anti-DR3 monoclonal antibodies 142A2 and 142S38B, respectively. Each line of sensorgram respectively indicates each concentration of Anti-DR3 antibody Fab fragments from 0.375 to 12 nM. The longitudinal axis indicates Fab binding (resonance unit) and the horizontal axis indicates the time after the Fab fragment injection.

FIG. 2A and FIG. 2B indicate comparison of isotype effects on anti-DR3 antibody agonist and antagonist activities, respectively. The effects of indicated 142A2 IgG1, IgG2, and IgG4v versions on PBMCs NF-kB activation were determined. The longitudinal axis indicates p65 phosphorylation-positive cells (%) in PBMCs and the horizontal axis indicates added antibodies. TL1A was added at 40 nM concentration and each antibody was added at 6.67 nM concentration.

FIG. 3A and FIG. 3B indicate an agonism (FIG. 3A) and an antagonism (FIG. 3B) on p65 phosphorylation level of anti-DR3 monoclonal antibodies 142A2 and 142S38B. In FIG. 3A, the longitudinal axis indicates p65 phosphorylation-positive cells (%) in PBMCs and the horizontal axis indicates antibody concentration (μg/mL). In FIG. 3B, the longitudinal axis indicates p65 phosphorylation-positive cells (%) in PBMCs and the horizontal axis indicates antibody concentration (μg/mL). 55% of cells were phosphorylated-p65 positive following TL1A treatment alone.

FIG. 4A and FIG. 4B indicate an agonism (FIG. 4A) and an antagonism (FIG. 4B) of IL-13 production by anti-DR3 monoclonal antibodies 142A2 and 142S38B. In FIG. 4A, the longitudinal axis indicates secreted IL-13 concentration (pg/mL) compared to secretion level by TL1A-flag as a positive control and the horizontal axis indicates antibody concentration (nM). In FIG. 4B, the longitudinal axis indicates secreted IL-13 concentration (pg/mL) in the presence of antibody+TL1A-flag (1 ug/mL) and the horizontal axis indicates antibody concentration (nM). IL-13 production levels were 1300 pg/mL and 750 pg/mL in the presence or absence of 1 μg/mL TL1A, respectively.

FIG. 5A and FIG. 5B indicate an agonism (FIG. 5A) and an antagonism (FIG. 5B) on p65 phosphorylation level of anti-DR3 monovalent antibody mv142A2. In FIG. 5A, the longitudinal axis indicates p65 phosphorylation-positive cells (%) in PBMCs and the horizontal axis indicates antibody concentration (nM). In FIG. 5B, the longitudinal axis indicates p65 phosphorylation-positive cells (%) in PBMCs and the horizontal axis indicates antibody concentration (μg/mL). 69.5% and 2.3% of cells were phosphorylated-p65 positive following TL A treatment alone or medium alone.

FIG. 6A and FIG. 6B indicate an agonism (FIG. 6A) and an antagonism (FIG. 6B) on p65 phosphorylation level of anti-DR3 monovalent antibody mv142S38B. In FIG. 6A, the longitudinal axis indicates p65 phosphorylation-positive cells (%) in PBMCs and the horizontal axis indicates antibody concentration (nM). In FIG. 6B, the longitudinal axis indicates p65 phosphorylation-positive cells (%) in PBMCs and the horizontal axis indicates antibody concentration (μg/mL). 45% and 3% of cells were phosphorylated-p65 positive following TL1A treatment alone or medium alone.

FIG. 7A and FIG. 7B indicate an agonism (FIG. 7A) and an antagonism (FIG. 7B) on p65 phosphorylation level of anti-DR3 antibody IgG2, IgG4 variant, IgG4244 variant and IgG2422 variant. In FIG. 7A, the longitudinal axis indicates p65 phosphorylation-positive cells (%) in PBMCs and the horizontal axis indicates antibody concentration (nM). In FIG. 7B, the longitudinal axis indicates p65 phosphorylation-positive cells (%) in PBMCs and the horizontal axis indicates antibody concentration (μg/mL). 55% and 1.8% of cells were phosphorylated-p65 positive following TL1A treatment alone or medium alone.

FIG. 8 indicates antagonism of IL-13 production by anti-DR3 monoclonal antibody Fabs 142A2 and 142A2-EQR. The longitudinal axis indicates secreted IL-13 concentration (pg/mL) in the presence of antibody+TL1A-flag (1 μg/mL) and the horizontal axis indicates antibody concentration (nM). All samples were costimulated with plate-bound anti-CD3 (10 μg/mL) and soluble anti-CD28 (1 μg/mL). IL-13 production levels were 1846 pg/mL and 974 pg/mL in the presence or absence of 1 μg/mL TL1A, respectively.

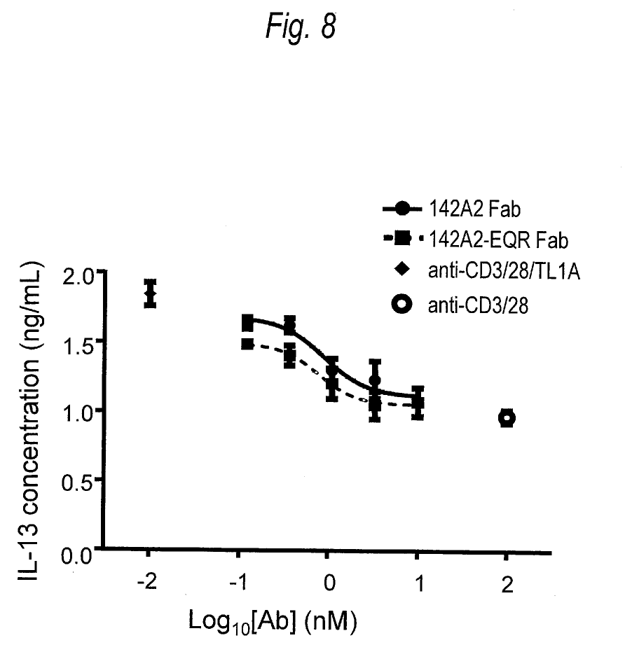

### DESCRIPTION OF EMBODIMENTS

### Definition

The term “antibody” as used herein, includes any antibodies such as a monoclonal antibody, an oligoclonal antibody and a polyclonal antibody.

The term “monoclonal antibody” as used herein, refers to an antibody which is constituted of a uniform amino acid sequence, in other words, a primary structure is the same. Further the monoclonal antibody recognizes only a single epitope (it's also called as a determinant of antigen).

The term “oligoclonal antibody” and “polyclonal antibody” as used herein, mean an antibody composition comprising plural antibodies more than two species of monoclonal antibody.

The term “antibody” as used herein, is also called immunoglobulin (hereinafter, referred to as Ig) and human antibody is classified into the isotypes of IgA1, IgA2, IgD, IgE, IgG1, IgG2, IgG3, IgG4 and IgM, based on the difference in its molecular structure. IgG1, IgG2, IgG3 and IgG4 having relatively high homology in amino acid sequences are generally called as IgG. Further the antibody of the present invention includes any antibody variants comprising amino acid sequences differed from amino acid sequences of a native antibody. The antibody of the present invention is preferably IgG antibody and its variants, more preferably an IgG2, an IgG2 variant comprising at least one domain originated from IgG2, an IgG2 variant comprising a hinge domain from IgG2, an IgG4 antibody variant comprising a hinge domain of IgG2, and an IgG4 antibody variant comprising a hinge domain of IgG2 and Lys is present in EU numbering position 409.

The term “native antibody” as used herein, refers an antibody naturally occurred in animals and have several amino acid sequences defined as allotypes.

The antibody molecule is composed of polypeptides, called a heavy chain (hereinafter, referred to as H chain) and a light chain (hereinafter, referred to as L chain).

Further, the H chain is constituted by regions of an H chain variable region (also referred to as VH) and an H chain constant region (also referred to as CH) from its N-terminus, and the L chain is constituted by regions of an L chain variable region (also referred to as VL) and an L chain constant region (also referred to as CL) from its N-terminus. Regarding CH, α, δ, ε, γ and chains are known for each subclass. Regarding CL, λ and κ are known. IgG antibodies have two heavy chains and two light chains, and form two antigen binding sites constituted of a VH and a VL. Therefore IgG antibodies are bivalent antibody.

A domain refers to a functional structural unit constituting each polypeptide of antibody molecules. Further, Fc and Fc region of the present invention refers to a partial sequence and a partial structure of H chain constant region composed of hinge domain, CH2 domain and CH3 domain.

Further, CH is composed of CH1 domain, hinge domain, CH2 domain and CH3 domain from the N-terminus. The CH1 domain, hinge domain, CH2 domain, CH3 domain, and Fc region in the present invention can be identified by the number of amino acid residues from the N-terminus according to the EU index [Kabat et al., Sequences of Proteins of Immunological Interest, US Dept. Health and Human Services (1991)]. A number of amino acid residue is followed by EU index by Kabat et al and in the present invention, a previous number of amino acid residue indicates original or parent residues of a polypeptide and an after number of amino acid residue indicates a replaced or substituted amino acid residues of the polypeptide.

Specifically, CH1 is identified by the amino acid sequence from positions 118 to 215 of the EU index, the hinge is identified by the amino acid sequence from positions 216 to 230 of the EU index, CH2 is identified by the amino acid sequence from positions 231 to 340 of the EU index, and CH3 is identified by the amino acid sequence from positions 341 to 447 of the EU index, respectively.

The term “recombinant antibody” as used herein, refers to recombinant antibodies produced by a recombination technology as well as monoclonal antibodies obtained from hybridomas. The recombinant antibodies include a chimeric antibody that is prepared by binding a human antibody constant region to a non-human antibody variable region, a humanized antibody (or CDR-grafted antibody) that is prepared by grafting the complementarity determining region (hereinafter, abbreviated to CDR) of H chain and L chain of a non-human antibody variable region into a framework region (hereinafter, abbreviated to FR) of a human antibody variable region, and a human antibody that is prepared by using a human antibody-producing animal, or the like.

The term “chimeric antibody” as used herein, refers to an antibody in which the amino acid sequence of VH and VL of a non-human animal antibody are grafted into the corresponding VH and VL of a human antibody. The chimeric antibody can be produced by obtaining cDNAs encoding VH and VL from a monoclonal antibody-producing hybridoma derived from a non-human animal, inserting them into an expression vector for animal cell having DNA encoding CH and CL of human antibody so as to construct a human chimeric antibody expression vector, and then introducing the vector into an animal cell so as to express the antibody.

The term “a humanized antibody” refers to an antibody in which the amino acid sequence of CDRs of VH and VL of a non-human animal antibody are grafted into the corresponding CDRs of VH and VL of a human antibody. The region other than CDRs of VH and VL is referred to as a framework region (hereinafter, referred to as FR).

The humanized antibody can be produced in the following manner: cDNA encoding an amino acid sequence of VH which consists of an amino acid sequence of CDR of VH of a non-human antibody and an amino acid sequence of FR of VH of any human antibody, and cDNA encoding an amino acid sequence of VL which consists of an amino acid sequence of CDR of VL of a non-human animal antibody and an amino acid sequence of FR of VL of any human antibody are constructed, these cDNAs are inserted respectively into expression vectors for animal cells having DNA encoding CH and CL of a human antibody so as to construct a humanized antibody expression vector, and this vector is introduced into animal cells so as to express the antibody.

The term “human antibody” as used herein, originally refers to an antibody naturally existing in the human body. However, the human antibody also includes antibodies that are obtained from a human antibody phage library, cloning of immortalized human peripheral blood lymphocytes, or human antibody-producing transgenic animals prepared according to the technical advancement in genetic engineering, cell engineering, and development engineering in recent years.

The human antibody can be obtained by immunizing a mouse having human immunoglobulin genes (Tomizuka K. et al., Proc Natl Acad Sci USA. 97, 722-7, 2000) with a desired antigen. In addition, by selecting a human antibody having a desired binding activity using a phage display library which is formed by antibody gene amplification from human B cells, it is possible to obtain human antibodies without performing immunization (Winter G. et al., Annu Rev Immunol. 12: 433-55. 1994).

Moreover, by immortalizing human B cells using an EB virus to prepare human antibody-producing cells having a desired binding activity, it is possible to obtain human antibodies (Rosen A. et al., Nature 267, 52-54. 1977).

The antibody existing in the human body can be purified in the following manner, for example; lymphocytes isolated from the human peripheral blood are immortalized by infection with the EB virus or the like, followed by cloning, whereby lymphocytes producing the antibody can be cultured and the antibody can be purified from the culture.

The human antibody phage library is a library of phages which are caused to express antibody fragments such as Fab and scFv on the surface thereof by insertion of antibody genes prepared from the human B cells into the gene of the phage. From this library, it is possible to recover phages which express antibody fragments having a desired antigen binding activity, by using binding activity with respect to an antigen-immobilized substrate as an index. The antibody fragments can be also converted into a human antibody molecule consisting of two complete H chains and two complete L chains by genetic engineering technique.

The human antibody-producing transgenic animal refers to an animal obtained by integration of the human antibody gene into chromosomes of a host animal. Specifically, the human antibody gene is introduced to mouse ES cells, the ES cells are grafted to the early embryo of another mouse, and then the embryo is developed, whereby the human antibody-producing transgenic animal can be prepared.

As a method of preparing human antibodies from the human antibody-producing transgenic animal, a human antibody-producing hybridoma is obtained by a normal hybridoma preparation method which is implemented using a mammal other than a human being, followed by culture, whereby human antibodies can be produced and accumulated in the culture.

Specifically, it can include amino acid sequences of VH and VL of a non-human animal antibody, a humanized antibody, and a human antibody that are produced by hybridomas or antibody-producing cells.

The amino acid sequence of CL in antibodies of the present invention can be any one of the amino acid sequence of human antibody or the amino acid sequence of non-human animal antibody. The amino acid sequence of Cκ or Cλ of human antibody is preferred.

CH in antibodies of the present invention can be any one belonging to immunoglobulin. Preferably, any of γ1(IgG1), γ2(IgG2), and γ4(IgG4) and their variants belongs to human IgG class, more preferably γ2(IgG2) and its variants can be used.

The term “antibody fragment” as used herein, refers to any antibody fragments which can bind to specific antigen DR3. For example, it includes Fab, Fab′, F(ab′)₂, single chain Fv (scFv), diabody, disulfide stabilized Fv (dsFv), a peptide comprising plural CDRs and a peptide comprising six CDRs of an antibody, further any Fc fusion proteins, such as Fab fused to a Fc region (Cater et al, Nature Med., 2006; 6; 343-357), scFv fused to a Fc region (Carter et al, Nature Med., 2006; 6; 343-357), a monovalent antibody in that one Fab is fused to a Fc region, a monovalent antibody composed of a H chain of an antibody and a L chain fused to a Fc region (hereinafter described as FL fusion polypeptide) (US2007/0105199) or the like (Labrjin et al, Curr. Opin in Immunol., 2008; 20; 479-485).

Fab refers to an antibody fragment having about a half H-chain of the N-terminus and a full L-chain which are bound to each other via a disulfide bond (S—S bond), a molecular weight of about 50000 and an antigen binding activity, among fragments (cleaved at position 224 of the amino acid residue of the H-chain) which are obtained by treating the IgG antibody with a protease papain.

F(ab′)₂ refers to an antibody fragment which is slightly longer than Fab bound to each other via a S—S bond of the hinge region and has a molecular weight of about 100,000 and an antigen binding activity, among fragments (cleaved at position 234 of the amino acid residue of the H-chain) which are obtained by treating IgG with a protease pepsin.

Fab′ is an antibody fragment which is obtained by cleaving the S—S bond of the hinge region of the F(ab′)₂ and has a molecular weight of about 50,000 and an antigen binding activity.

scFv is an antibody fragment having an antigen binding activity, which is a VH-P-VL or VL-P-VH polypeptide obtained by linking one VH to one VL by using an appropriate peptide linker (P), such as a linker peptide prepared by linking an arbitrary number of linker (G4S) consisting of 4 Gly residues and 1 Ser residue.

Diabody is an antibody fragment as a dimer formed of scFvs showing the same or different antigen binding specificity, and this antibody fragment has a divalent antigen binding activity with respect to the same antigen or has 2 types of specific antigen binding activity with respect to different types of antigens.

dsFv is one in which 1 amino acid residue in each of VH and VL is substituted with a cystine residue, and the polypeptides are linked through a S—S bond between these cysteine residues.

The peptide comprising CDR is constituted with at least one or more regions of CDR of VH or VL. In the peptide comprising plural CDRs, the CDRs can be bound to each other directly or via an appropriate peptide linker.

It can be prepared by constructing DNAs encoding CDRs of VH and VL of the antibody of the present invention, inserting these DNAs into an expression vector for prokaryote or eukaryote, and introducing this expression vector into prokaryote or eukaryote for expression. The peptide comprising CDR can be also prepared by chemical synthesis method such as an Fmoc method or a tBoc method.

The Fc fusion protein such as Fab-Fc, scFv-Fc, (Fab)₂-Fc, a monovalent antibody in that one Fab is fused to Fc region and the monovalent antibody composed of the H chain of the antibody and the FL fusion polypeptide can be produced by preparing an amino acid sequence fused a required fragment derived from the antibody with the Fc region, constructing a cDNA encoding the Fc fusion protein into an expression vector and expressing the Fe fusion protein in an appropriate host cells.

The term “variant” as used herein refers to a polypeptide that retains similar or identical activity as DR3 polypeptide, DR3 polypeptide fragment thereof, an antibody or antibody fragment thereof, however has similar amino acid sequence or different amino acid sequence compared to the parent antibody or antibody fragment thereof (or original antibody or antibody fragment thereof). The antibody variant having similar amino acid sequence refers to a polypeptide comprising an amino acid sequence at least 50%, at least 60%, at least 70%, at least 75%, preferably at least 80%, at least 85%, more preferably at least 90%, 91%, 92%, 93%, 94%, 95%, 96%, 97%, 98%, or 99% identical to the amino acid sequence of variable region such as VH or VL, or CDRs of an anti-DR3 antibody or antibody fragment thereof. Furthermore, the variant of the present invention includes any variant that comprises at least one amino acid substitution in a constant region of the antibody. The antibody variant also includes any domain exchanged (or domain swapped) antibody in each domain of a constant region of an antibody such as CH1, hinge, CH2 and CH3 domains including or not including at least one amino acid substitution. More preferably an antibody variant includes an IgG2, an IgG2 variant comprising at least one domain originated from IgG2, an IgG2 variant comprising domains originated from IgG2 and IgG4 antibodies, an IgG2 variant comprising a hinge domain from IgG2 and CH1, CH2 and CH3 domains from IgG4, and an IgG2 variant comprising a hinge domain from IgG2 and CH1, CH2 and CH3 domains from CH3 domains from IgG4 wherein the amino acid residue at EU numbering position 409 in the CH3 domain is Lys.

The term “epitope” as used herein, refers to any amino acid sequences and any three dimensional structures localized on the surface of an antigen recognized and/or bound by an antibody. For example, it is included that a single amino acid sequence recognized and bound by a monoclonal antibody, a conformation of the amino acid sequence, an amino acid sequence bound with a modification residue such as a sugar chain, an amino group, a carboxyl group, phosphate, sulfate or the like, and a conformation of the amino acid sequence bound with the modification residue. The conformation is a naturally occurring three-dimensional structure of a protein, and it refers to a conformation of proteins that are expressed within cells or on plasma membrane of cells.

The epitope of the present invention can be a linear epitope constituted from continuous amino acid sequence, a non-continuous amino acid sequence or a conformational structure of DR3 polypeptide. In one embodiment, the epitope of the present invention is an epitope comprising one or more amino acid residues existed in an extracellular region of DR3 polypeptide. In other embodiment, the epitope is an epitope existed in a molecular surface of DR3 bound to TL1A ligand.

The term “death receptor 3”, “DR3”, “DR3 peptide”, “DR3 protein” or “DR3 polypeptide”, as used herein, refers to a polypeptide comprising an amino acid sequence of SEQ ID NO:4; a polypeptide comprising an amino acid sequence of Uniplot No. Q93038, a polypeptide comprising an amino acid sequence in which one or more amino acid residue(s) is/are deleted, substituted or added in the amino acid sequence represented by SEQ ID NO2: or Uniplot No. Q93038, and having the activity of DR3; a polypeptide comprising an amino acid sequence having at least 60% homology, preferably at least 80% homology, more preferably at least 90% homology, and most preferably at least 95%, 96%, 97%, 98% or 99% homology, with the amino acid sequence represented by SEQ ID NO:3 or Uniplot No. Q93038, and having the activity of DR3; and related polypeptides including SNP variants and the like. The related polypeptides include SNPs variants, splice variants, fragments, substitution, deletion, and insertion, preferably which retain DR3 activities/functions. Tumor necrosis factor receptor super-family 25 (TNFRSF25), lymphocyte-associated receptor of death (LARD), APO-3, TRAMP and WSL-1 are also known as synonyms of DR3, these are necessarily same as DR3.

Further a polypeptide encoded by a nucleotide sequence of SEQ ID NO:3 or NM_003790.2. As the gene encoding DR3, the gene encoding DR3 of the present invention also included a gene containing a DNA comprising a nucleotide sequence having deletion(s), substitution(s) or addition(s) of one or more nucleotides in the nucleotide sequence of SEQ ID NO:3 or NM_003790.2 and also encoding a polypeptide having the function of DR3; a gene containing a DNA consisting of a nucleotide sequence having at least 60% or higher homology, preferably 80% or higher homology, and more preferably 95%, 96%, 97%, 98% or 99%, higher homology, with the nucleotide sequence of SEQ ID NO:3 or NM_003790.2, and also encoding a polypeptide having the function of DR3; a gene consisting of a DNA which hybridizes with a DNA having the nucleotide sequence of SEQ ID NO:3 or NM_003790.2 under stringent conditions and also containing a DNA that encodes a polypeptide having the function of DR3; or the like.

The polypeptide comprising an amino acid sequence in which one or more amino acid residue(s) is/are deleted, substituted and/or added in the amino acid sequence of SEQ ID NO:4 or Uniplot No. Q93038 can be obtained, for example, by introducing a site-specific mutation into DNA encoding a polypeptide comprising the amino acid sequence of SEQ ID NO:4 by site-specific mutagenesis [Molecular Cloning, A Laboratory Manual, Second Edition, Cold Spring Harbor Laboratory Press (1989), Current Protocols in Molecular Biology, John Wiley & Sons (1987-1997), Nucleic Acids Research, 10, 6487 (1982), Proc. Natl. Acad. Sci. USA, 79, 6409 (1982), Gene, 34, 315 (1985), Nucleic Acids Research, 13, 4431 (1985), or Proc. Natl. Acad. Sci. USA, 82, 488 (1985)] or the like. The number of amino acid residues which are deleted, substituted or added is not particularly limited, and the number is preferably, 1 to dozens, such as 1 to 20, and more preferably 1 to several, such as 1 to 5.

The term “the DNA which hybridizes under stringent conditions” as used herein, refers to a DNA which is obtained by colony hybridization, plaque hybridization, Southern blot hybridization, DNA microarray or the like using a DNA having the nucleotide sequence of SEQ ID NO:3 or NM_003790.2 as a probe. A specific example of such DNA is a hybridized colony- or plaque derived DNA which can be identified by performing hybridization at 65° C. in the presence of 0.7 to 1.0 mol/L sodium chloride using a filter or slide glass with the PCR product or oligo DNA having immobilized thereon, and then washing the filter or slide glass at 65° C. with a 0.1 to 2-fold concentration SSC solution (1-fold concentration SSC solution: 150 mmol/L sodium chloride and 15 mmol/L sodium citrate). Hybridization can be carried out according to the methods [Molecular Cloning, A Laboratory Manual, Second Edition, Cold Spring Harbor Lab. Press (1989), Current Protocols in Molecular Biology, John Wiley & Sons (1987-1997); DNA Cloning 1: Core Techniques, A Practical Approach, Second Edition, Oxford University (1995)] and the like. Specifically, the DNA capable of hybridization includes DNA having at least 60% or more homology, preferably 80% or more homology, more preferably 90% or more homology, and most preferably 95%, 96%, 97%, 98% or 99% or more homology to the nucleotide sequence of SEQ ID NO:3 or NM_003790.2.

In the nucleotide sequence of the gene encoding a protein of a eukaryote, genetic polymorphism is often recognized. The DR3 gene used in the present invention also includes a gene in which small modification is generated in the nucleotide sequence by such polymorphism as the gene used in the present invention.

The number of the homology in the present invention can be a number calculated by using a homology search program known by the skilled person, unless otherwise indicated. Regarding the nucleotide sequence, the number can be calculated by using BLAST [J. Mol. Biol., 215, 403 (1990)] with a default parameter or the like, and regarding the amino acid sequence, the number may be calculated by using BLAST2 [Nucleic Acids Res., 25, 3389 (1997); Genome Res., 7, 649 (1997) with a default parameter, http://www.ncbi.nlm.nih.gov/Education/BLASTinfo/information3.html] or the like.

As the default parameter, G (cost to open gap) is 5 for the nucleotide sequence and 11 for the amino acid sequence; -E (cost to extend gap) is 2 for the nucleotide sequence and 1 for the amino acid sequence; -q (penalty for nucleotide mismatch) is -3; -r (reward for nucleotide match) is 1; -e (expect value) is 10; -W (wordsize) is 11 residues for the nucleotide sequence and 3 residues for the amino acid sequence; -y [dropoff (X) for blast extensions in bits] is 20 for blastn and 7 for a program other than blastn; -X (X dropoff value for gapped alignment in bits) is 15; and -Z (final X dropoff value for gapped alignment in bits) is 50 for blastn and 25 for a program other than blastn (http://www.ncbi.nlm.nih.gov/blast/html/blastcgihelp.html).

The polypeptide comprising a partial sequence of the amino acid sequence of SEQ ID NO:4 or Uniplot No. Q93038 can be prepared according to a method known by the skilled person. For example, it can be prepared by deleting a part of DNA encoding the amino acid sequence of SEQ ID NO:2 and culturing a transformant into which an expression vector containing the DNA is introduced. Also, based on the polypeptide or DNA prepared by using the above method, a polypeptide comprising an amino acid sequence in which one or more amino acid(s) is/are deleted, substituted or added in a partial sequence of the amino acid sequence of SEQ ID NO:4 or Uniplot No. Q93038 can be prepared in the same manner as described above. In addition, the polypeptide comprising a partial sequence of the amino acid sequence of SEQ ID NO:4 or Uniplot No. Q93038; or a polypeptide comprising an amino acid sequence in which one or more amino acid(s) is/are deleted, substituted or added in a partial sequence of the amino acid sequence of SEQ ID NO:4 or Uniplot No. Q93038 can be produced by a chemical synthesis method such as fluorenylmethoxycarbonyl (Fmoc) method or t butyloxycarbonyl (tBoc) method.

In the present invention, the extracellular region of DR3 includes, for example, regions predicted from the amino acid sequence of the polypeptide represented by SEQ ID NO:3 by using conventionally known transmembrane region deducing program SOSUI (http://bp.nuap.nagoya-u.ac.jp/SOSUI/SOSUI_submit.), TMHMM ver. 2 (http://www.cbs.dtu.dk/servicesfIMHMM-2.0/), ExPASy Proteomics Server (http://Ca.expasy.org/) or the like.

Examples of the extracellular region of DR3 in the present invention include regions corresponding to positions 25 to 201 in the extracellular domain. DR3 comprises four cysteine rich domains (CRDs) of CRD1 to CRD4 in the extracellular domain, followed transmembrane domain, topological domain and death domain in the intracellular domain.

Each CRD generally contains six cysteine residues that form three disulfide bonds at the interface of the domain.

The activity/function of DR3 refers that DR3 induces the activation and proliferation of T cells, by an engaging of TL1A ligand. DR3 activation also induces cytokine release from T cells, including interleukin (IL)-13, IL-17, GM-CSF and IFN-γ through NF-κB phosphorylation. Further DR3 activation exhibits apoptosis of some cell types.

The term “agonism”, “agonistic activity”, “agonist potency” or “agonistic function” against DR3 refers to activate or stimulate DR3 by a ligand or an antibody bound. Accordingly these binders can induce NF-κB phosphorylation, the activation and proliferation of T cells, apoptosis of DR3 positive cells and cytokine release from T cells such as interleukin (IL)-13, IL-17, GM-CSF and IFN-γ.

The term “antagonism”, “antagonistic activity”, “antagonist potency” or “antagonistic function” against DR3 refers to inhibit or prevent an activity of DR3 caused by TL1A ligand bound. Accordingly antagonists having the property can inhibit phosphorylation of DR3 and NF-κB, the activation, proliferation, infiltration of T cells, apoptosis of DR3 positive cells and cytokine releases from T cells such as interleukin (IL)-13, IL-17, GM-CSF and IFN-γ, caused by TL1A ligand bound.

The term “decreased, lowered, reduced/deleted, canceled, no agonistic activity for DR3” of the present invention refers to antibodies of the present invention display minimum agonism induced by themselves binding, antibodies of the present invention don't essentially activate, stimulate or agonize DR3 by themselves binding or antibodies of the present invention don't activate, stimulate or agonize DR3 by themselves binding. In one particular embodiment of the present invention, it refers that an antibody doesn't essentially induce phosphorylation of NF-κB and/or p65 subunit of NF-κB in peripheral blood mononuclear cells (PBMCs), an antibody doesn't essentially induce cytokine release in DR3 expressed cells or PBMCs, and an antibody doesn't essentially induce T cell proliferation. In one particular and preferable embodiment, it refers that an antibody induces phosphorylation of p65 subunit of NF-κB less than 20%, preferably 10% cells compared to total cells in PBMCs.

### Anti-Death Receptor 3 (DR3) Antibodies

The present invention provides that an anti-DR3 antagonistic IgG antibodies and variants thereof which have decreased or no agonistic activity, a method for producing the antibodies thereof, amino acid sequences of antibodies thereof, a nucleotide sequences encoding antibody, a vector comprising nucleotide sequences encoding antibody, a method of decreasing agonistic activity of antagonistic IgG antibody and a method of treating diseases comprises administration of the antibody. Furthermore, the present invention provides that antibody fragments derived from antibodies described in the above, a method for producing the antibody fragments thereof, amino acid sequences of antibody fragment thereof, a nucleotide sequences encoding antibody fragment, a vector comprising nucleotide sequences encoding antibody fragment, a method of decreasing agonistic activity of antagonistic IgG antibody fragment and a method of treating diseases comprises administration of the antibody fragment.

The anti-DR3 antibody of the present invention can bind to an extracellular region of DR3 polypeptide and block/neutralize an activity of the DR3 polypeptide, inhibit T cell proliferation, because the antibody itself hardly activate DR3 or doesn't activate DR3. Accordingly the antibody of the present invention hardly activate DR3 or doesn't activate DR3 by own binding, in other word, the antibody of the present invention has decreased/no agonistic activity for DR3. In one embodiment, antibodies of the present invention include antibodies that have a neutralizing/blocking activity of DR3 and have decreased or no agonistic activity, antibodies that can bind to the extracellular region of DR3, neutralize the activity of DR3 and have decreased or no agonistic activity. The extracellular region of DR3 is divided into four cysteine-rich domains (CRDs) from N-terminal of a polypeptide, and N-terminal region comprising CRD1 domain has been known as a pre-ligand assembly domain (known as “PLAD”), the PLAD domain plays role to form trimers consisting of three DR3 monomers. Accordingly the anti-DR3 antibody of the invention binds to at least one monomer of DR3, monomers of DR3, and/or trimeric DR3 (trimers).

In one embodiment, the present invention provides antibodies which bind to at least one epitope included in the extracellular region of DR3, inhibit the formation of DR3 trimers and neutralize DR3 activation through TL1A binding, wherein the antibodies exhibit a decreased/on agonistic activity for DR3. In one embodiment, the present invention provides antibodies which bind to at least one epitope included in the extracellular region of DR3, block TL1A bound to DR3 and neutralize DR3 activation through TL1A binding, wherein the antibodies have a decreased/on agonistic activity for DR3. In preferable embodiments of the present invention, antibodies bind to PLAD domain, CRD1 domain, CRD2 domain, CRD3 domain, CRD4 domain or inter domain of CRD3 and CRD4, inhibit the formation of DR3 trimers and neutralize DR3 activation through TL1A binding, wherein the antibodies exhibit a decreased/no agonistic activity for DR3.

In preferable embodiments of the present invention, antibodies or antibody fragments bind to an epitope comprising amino acid residue present in at least one domain selected from PLAD domain, CRD1 domain, CRD2 domain, CRD3 domain, CRD4 domain and inter domain of CRD3 and CRD4, neutralize DR3 activation through TL1A binding, wherein the antibodies have a decreased/no agonistic activity for DR3, antibodies bind to an epitope present in at least one domain selected from PLAD domain, CRD1 domain, CRD2 domain, CRD3 domain, CRD4 domain and inter domain of CRD3 and CRD4, inhibit the formation of DR3 trimers and neutralize DR3 activation through TL1A binding, wherein the antibodies have a decreased/no agonistic activity for DR3.

In other embodiments of the present invention, antibodies bind to a surface of DR3 bound with TL1A, or a surface of DR3 involved in trimerization of DR3, and neutralize DR3 activation through TL1A binding, wherein the antibodies exhibit a decreased/no agonistic activity for DR3. In one preferable embodiment, antibodies or antibody fragments of the present invention include an antibody bound to PLAD domain or CRD1 domain, blocks TL1A binding, and neutralizes DR3 activation through TL1A binding, wherein the antibody has a decreased/no agonistic activity for DR3. In another preferable embodiment, antibodies of the present invention include an antibody bound to CRD4 domain blocks TL1A binding, and neutralizes DR3 activation through TL1A binding, wherein the antibody has a decreased/no agonistic activity for DR3.

In more preferable embodiment, antibodies or antibody fragments of the present invention include an antibody bound to the epitope comprising at least one amino acid residue present in the CRD1 domain, the amino acid sequence of 47 to 71 position of SEQ ID NO:4, or the epitope comprising at least one amino acid residue present in the amino acid sequence of 117 to 123 position and 140 to 170 position of SEQ ID NO:4, wherein the antibody neutralizes DR3 activation through TL1A binding and exhibits a decreased or no agonistic activity for DR3. Further antibodies or antibody fragments of the present invention include an antibody competitively bound to DR3 with an another particular antibody described in the above, an antibody bound to an epitope included in the epitope recognized by an another particular antibody described in the above, and an antibody bound to the same epitope bound by an another particular antibody described in the above.

In one preferable embodiment of the present invention, antibodies or antibody fragments neutralize the activity of DR3 and have decreased or no agonistic activity, wherein the antibodies comprise at least a part of an amino acid sequence of a CH1 domain and/or a hinge domain derived from IgG2 class and variants thereof. The part of the amino acid sequence of the CH1 domain and/or the hinge domain derived from IgG2 subclass in the present invention can include any part of region of them as far as an antibody in that the part of the amino acid sequence of CH1 domain and/or the hinge domain derived from IgG2 subclass is comprised, has a decreased or no agonistic activity for DR3. The part of amino acid sequence can include any amino acid sequences such as a continuous sequence, a sporadic or discontinuous sequence. The antibody in that the CH1 domain and/or the hinge domain derived from IgG2 of the present invention has decreased or no agonistic activity for DR3, even if the antibody can neutralize the activity of DR3 by TL1A binding. Accordingly the antibody in that the CH1 domain and/or the hinge domain derived from IgG2 of the present invention specifically antagonizes, blocks, or neutralizes DR3 activity with decreased or deleted agonism for DR3.

In one preferable embodiment of the present invention, antibodies or antibody fragments neutralize the activity of DR3 and have decreased or no agonistic activity, wherein the antibodies are IgG2 class and variants thereof. The IgG2 class antibody of the present invention has decreased or no agonistic activity for DR3, even if the antibody can neutralize the activity of DR3 by TL1A binding. Accordingly the antibody of the present invention specifically antagonizes, blocks, or neutralizes DR3 activity with decreased or deleted agonism for DR3, although all known IgG antibodies have neutralized DR3 activity and have agonistic activity by own binding. Namely, because the agonism of antibody of the present invention is decreased or canceled, the antibody purely or only has antagonism for DR3. The property of the antibody has great merit in order to treat patients suffered from DR3 related diseases by administering anti-DR3 antibody, because it can be considered that adverse events or lowered therapeutic effects are caused by agonism of the antibody. In another embodiment, an antibody of the present invention includes any Gm allotypes such as G2(n+ or n−) and G2m (23), and the like, all existed in the nature, for human IgG2 (Hougs et al, Immunogenetics, 2001: 52, 242-248, Brusco et al, Imunogenetics, 1995; 42: 414-417).

In another preferable embodiment of the present invention, antibodies or antibody fragments neutralize the activity of DR3 and have decreased or no agonistic activity, wherein the antibodies are IgG2 variants comprising at least one amino acid substitution selected from 234, 235, 237, 250, 300, 309, 339, 331 and 428 positions in the Fc region of the antibody. In more preferable embodiment, antibodies neutralize the activity of DR3 and have decreased or no agonistic activity, wherein the antibody are selected from 1) the antibody comprising at least one amino acid substitution selected from V234A, L235E, G237A, T250Q, F300Y, V309L, T339A, P331S and M428L in the Fc region of human IgG2 antibody, 2) an antibody comprising amino acid substitutions of T250Q and M428L in the Fc region of human IgG2 antibody, 3) an antibody comprising amino acid substitutions of V234A, G237A and P331S in the Fc region of human IgG2 antibody, 4) an antibody comprising amino acid substitutions of F300Y, V309L and T339A in the Fc region of human IgG2 antibody, and 5) an antibody comprising amino acid substitutions of V234A, L235E, G237A, T250Q, F300Y, V309L, T339A, P331S and M428L in the Fc region of human IgG2 antibody or the like.

In another preferable embodiment of the present invention, antibodies or antibody fragments comprises a hinge domain of IgG2 class antibody, antibodies or antibody fragments comprises a hinge domain of IgG2 class antibody, and CH1, CH2 and CH3 domains of IgG4 antibody, and antibodies or antibody fragments comprises a hinge domain of IgG2 class antibody, and CH1, CH2 and CH3 domains of IgG4 antibody, wherein the amino acid residue at EU numbering position 409 in CH3 domain is Lys.

In another preferable embodiment of the present invention, antibodies or antibody fragments comprises an IgG4 antibody variant comprising a hinge domain of IgG2, and an IgG4 antibody variant comprising a hinge domain of IgG2 and Lys is present in EU numbering position 409.

In particular embodiment, antibodies of the present invention include an anti-DR3 monoclonal antibodies 142A2, 142S38B, and their antibody variants, an antibody comprising amino acid sequences of CDR1 to CDR3 of VH of SEQ ID NOs:16-18 and amino acid sequences of CDR1 to CDR3 of VL of SEQ ID NOs:22-24, an antibody comprising amino acid sequences of CDR1 to CDR3 of VH of SEQ ID NOs:28-30 and amino acid sequences of CDR1 to CDR3 of VL of SEQ ID NOs:34-36, an antibody comprising amino acid sequences of CDR1 to CDR3 of VH of SEQ ID NOs:16, 17, 80 and amino acid sequences of CDR1 to CDR3 of VL of SEQ ID NOs:22-24, an antibody comprising amino acid sequences of CDR1 to CDR3 of VH of SEQ ID NOs:16, 17, 80 and amino acid sequences of CDR1 to CDR3 of VL of SEQ ID NOs:22, 81, 24, an antibody comprising amino acid sequences of CDR1 to CDR3 of VH of SEQ ID NOs: 16, 17, 80 and amino acid sequences of CDR1 to CDR3 of VL of SEQ ID NOs:22, 82, 24, an antibody comprising amino acid sequences of CDR1 to CDR3 of VH of SEQ ID NOs:16-18 and amino acid sequences of CDR1 to CDR3 of VL of SEQ ID NOs:22, 81, 24, an antibody comprising amino acid sequences of CDR1 to CDR3 of VH of SEQ ID NOs: 16-18 and amino acid sequences of CDR1 to CDR3 of VL of SEQ ID NOs:22, 82, 24, an antibody comprising amino acid sequences of CDR1 to CDR3 of VH of SEQ ID NOs:16, 17, 80 and amino acid sequences of CDR1 to CDR3 of VL of SEQ ID NOs:22, 83, 24, an antibody comprising an amino acid sequence of VH of SEQ ID NO:15 and an amino acid sequence of VL of SEQ ID NO:21, an antibody comprising an amino acid sequence of VH of SEQ ID NO:76 and an amino acid sequence of VL of SEQ ID NO:21, an antibody comprising an amino acid sequence of VH of SEQ ID NO:15 and an amino acid sequence of VL of SEQ ID NO:77, an antibody comprising an amino acid sequence of VH of SEQ ID NO:15 and an amino acid sequence of VL of SEQ ID NO:78, an antibody comprising an amino acid sequence of VH of SEQ ID NO:15 and an amino acid sequence of VL of SEQ ID NO:79, an antibody comprising an amino acid sequence of VH of SEQ ID NO:76 and an amino acid sequence of VL of SEQ ID NO:79, an antibody comprising an amino acid sequence of VH of SEQ ID NO:27 and an amino acid sequence of VL of SEQ ID NO:33.

In other embodiment, antibodies of the present invention includes an antibody comprising at least 90%, preferably at least 91%, 92%, 93%, 94%, more preferably at least 95%, 96%, 97%, 98% or 99% identical to the each amino acid sequence of VH of SEQ ID NO:15 and VL of SEQ ID NO:21, VH of SEQ ID NO:76 and VL of SEQ ID NO:21, VH of SEQ ID NO:15 and VL of SEQ ID NO:77, VH of SEQ ID NO:15 and VL of SEQ ID NO:78, VH of SEQ ID NO:15 and VL of SEQ ID NO:79, VH of SEQ ID NO:76 and VL of SEQ ID NO:79 or VH of SEQ ID NO:27 and VL of SEQ ID NO:33, and an IgG2 antibody comprising at least 95%, 96%, 97%, 98% or 99% identical to the each amino acid sequences of CDRs of VH of SEQ ID NOs:16-18 and CDRs of VL of SEQ ID NOs:22-24, CDRs of VH of SEQ ID NOs:16, 80, 18 and CDRs of VL of SEQ ID NOs:22-24, CDRs of VH of SEQ ID NOs:16-18 and CDRs of VL of SEQ ID NOs:22, 81, 24, CDRs of VH of SEQ ID NOs:16-18 and CDRs of VL of SEQ ID NOs:22, 82, 24, CDRs of VH of SEQ ID NOs:16-18 and CDRs of VL of SEQ ID NOs:22, 83, 24, CDRs of VH of SEQ ID NOs:16, 80, 18 and CDRs of VL of SEQ ID NOs:22, 81, 24, CDRs of VH of SEQ ID NOs:16, 80, 18 and CDRs of VL of SEQ ID NOs:22, 82, 24, CDRs of VH of SEQ ID NOs:16, 80, 18 and CDRs of VL of SEQ ID NOs:22, 83, 24, or VH of CDRs of SEQ ID NOs:28-30 and CDRs of VL of SEQ ID NOs:34-36. Moreover antibodies of the present invention also include any affinity matured antibody clone being obtained from any kind screening method.

In other embodiment, antibodies of the present invention include an antibody bound to an epitope recognized by an anti-DR3 monoclonal antibodies 142A2, 142S38B or their antibody variants, an antibody comprising amino acid sequences of CDR1 to CDR3 of VH of SEQ ID NOs:16-18 and amino acid sequences of CDR1 to CDR3 of VL of SEQ ID NOs:22-24, an antibody comprising amino acid sequences of CDRs of VH of SEQ ID NOs:16, 80, 18 and CDRs of VL of SEQ ID NOs:22-24, CDRs of VH of SEQ ID NOs:16-18 and CDRs of VL of SEQ ID NOs:22, 81, 24, an antibody comprising amino acid sequences of CDRs of VH of SEQ ID NOs:16-18 and CDRs of VL of SEQ ID NOs:22, 82, 24, an antibody comprising amino acid sequences of CDRs of VH of SEQ ID NOs:16-18 and CDRs of VL of SEQ ID NOs:22, 83, 24, an antibody comprising amino acid sequences of CDRs of VH of SEQ ID NOs:16, 80, 18 and CDRs of VL of SEQ ID NOs:22, 81, 24, an antibody comprising amino acid sequences of CDRs of VH of SEQ ID NOs:16, 80, 18 and CDRs of VL of SEQ ID NOs:22, 82, 24, an antibody comprising amino acid sequences of CDRs of VH of SEQ ID NOs:16, 80, 18 and CDRs of VL of SEQ ID NOs:22, 83, 24, an antibody comprising amino acid sequences of CDR1 to CDR3 of VH of SEQ ID NOs:28-30 and amino acid sequences of CDR1 to CDR3 of VL of SEQ ID NOs:34-36, an antibody comprising an amino acid sequence of VH of SEQ ID NO:15 and an amino acid sequence of VL of SEQ ID NO:21, an antibody comprising amino acid sequences of VH of SEQ ID NO:76 and VL of SEQ ID NO:21, an antibody comprising amino acid sequences of VH of SEQ ID NO:15 and VL of SEQ ID NO:77, an antibody comprising amino acid sequences of VH of SEQ ID NO:15 and VL of SEQ ID NO:78, an antibody comprising amino acid sequences of VH of SEQ ID NO:15 and VL of SEQ ID NO:79, an antibody comprising amino acid sequences of VH of SEQ ID NO:76 and VL of SEQ ID NO:79 or an antibody comprising an amino acid sequence of VH of SEQ ID NO:27 and an amino acid sequence of VL of SEQ ID NO:33. Further antibodies of the present invention include an antibody competitively bound to DR3 with an another particular antibody described in the above.

In more particular embodiment, antibodies of the present invention include an anti-DR3 monoclonal antibodies 142A2, 142S38B and their antibody variants that are rearranged to IgG2 subclass, IgG2 variant comprising a hinge domain of IgG2, IgG2 variant comprising a hinge domain of IgG2 and CH1, CH2 and CH3 domain of IgG4, an IgG2 antibody comprising amino acid sequences of CDR1 to CDR3 of VH of SEQ ID NOs:16-18 and amino acid sequences of CDR1 to CDR3 of VL of SEQ ID NOs:22-24, an antibody comprising amino acid sequences of CDR1 to CDR3 of VH of SEQ ID NOs:28-30 and amino acid sequences of CDR1 to CDR3 of VL of SEQ ID NOs:34-36, an IgG2 antibody comprising an amino acid sequence of VH of SEQ ID NO:15 and an amino acid sequence of VL of SEQ ID NO:21, an IgG2 antibody comprising an amino acid sequence of VH of SEQ ID NO:27 and an amino acid sequence of VL of SEQ ID NO:33. In other embodiment, antibodies of the present invention includes an IgG2 antibody comprising at least 90%, preferably at least 91%, 92%, 93%, 94%, more preferably at least 95%, 96%, 97%, 98% or 99% identical to the each amino acid sequence of VH of SEQ ID NO:15 and VL of SEQ ID NO:21, or VH of SEQ ID NO:27 and VL of SEQ ID NO:33, and an IgG2 antibody comprising at least 95%, 96%, 97%, 98% or 99% identical to the each amino acid sequences of CDRs of VH of SEQ ID NOs:16-18 and CDRs of VL of SEQ ID NOs:22-24, or VH of CDRs of SEQ ID NOs:28-30 and CDRs of VL of SEQ ID NOs:34-36. Moreover antibodies of the present invention also include any affinity matured antibody clone being obtained from any kind screening method. For example, an antibody comprising amino acid sequences of CDRs of VH of SEQ ID NOs:16, 80, 18 and CDRs of VL of SEQ ID NOs:22-24, CDRs of VH of SEQ ID NOs:16-18 and CDRs of VL of SEQ ID NOs:22, 81, 24, an antibody comprising amino acid sequences of CDRs of VH of SEQ ID NOs:16-18 and CDRs of VL of SEQ ID NOs:22, 82, 24, an antibody comprising amino acid sequences of CDRs of VH of SEQ ID NOs:16-18 and CDRs of VL of SEQ ID NOs:22, 83, 24, an antibody comprising amino acid sequences of CDRs of VH of SEQ ID NOs:16, 80, 18 and CDRs of VL of SEQ ID NOs:22, 81, 24, an antibody comprising amino acid sequences of CDRs of VH of SEQ ID NOs:16, 80, 18 and CDRs of VL of SEQ ID NOs:22, 82, 24, an antibody comprising amino acid sequences of CDRs of VH of SEQ ID NOs:16, 80, 18 and CDRs of VL of SEQ ID NOs:22, 83, 24, an antibody comprising amino acid sequences of VH of SEQ ID NO:76 and VL of SEQ ID NO:21, an antibody comprising amino acid sequences of VH of SEQ ID NO:15 and VL of SEQ ID NO:77, an antibody comprising amino acid sequences of VH of SEQ ID NO:15 and VL of SEQ ID NO:78, an antibody comprising amino acid sequences of VH of SEQ ID NO:15 and VL of SEQ ID NO:79, and an antibody comprising amino acid sequences of VH of SEQ ID NO:76 and VL of SEQ ID NO:79 are included in the present invention.

In other embodiment, antibodies of the present invention include an IgG2 antibody and IgG2 variant bound to an epitope recognized by an anti-DR3 monoclonal antibodies 142A2, 142S38B, an IgG2 antibody comprising amino acid sequences of CDR1 to CDR3 of VH of SEQ ID NOs:16-18 and amino acid sequences of CDR1 to CDR3 of VL of SEQ ID NOs:22-24, an IgG2 antibody comprising amino acid sequences of CDR1 to CDR3 of VH of SEQ ID NOs:28-30 and amino acid sequences of CDR1 to CDR3 of VL of SEQ ID NOs:34-36, an IgG2 antibody comprising an amino acid sequence of VH of SEQ ID NO:15 and an amino acid sequence of VL of SEQ ID NO:21, or an IgG2 antibody comprising an amino acid sequence of VH of SEQ ID NO:27 and an amino acid sequence of VL of SEQ ID NO:33. Further antibodies of the present invention include an IgG2 antibody competitively bound to DR3 with another particular antibody described in the above.

In one embodiment, antibody fragments of the present invention include Fab, Fab′, F(ab′)₂, single chain Fv (scFv), diabody, disulfide stabilized Fv (dsFv), a peptide comprising plural CDRs, a peptide comprising six CDRs of an antibody and Fc fusion proteins such as the monovalent antibody in that one Fab is fused to a Fc region (Fab-Fc), the bivalent antibody in that two Fab are fused to a Fc region [(Fab)₂-Fc], the monovalent antibody in that one scFv is fused to a Fc region (scFv-Fc), the bivalent antibody in that two scFv are fused to a Fc region [(scFv)₂-Fc], the monovalent antibody composed of a H chain of an antibody and Fc fused L chain (hereinafter described as “FL”) or the like. The antibody fragments of the present invention can antagonize TL1A induced DR3 activity, wherein the antibody fragment has decreased or no agonistic activity.

In one preferable embodiment of the present invention, antibody fragments include monovalent antibodies and bivalent antibodies that have one or two antigen binding domain composed of VH and VL, such as Fab or scFv. In one more preferable embodiment of the present invention, monovalent antibodies include the monovalent antibody in that one Fab is fused to a Fc region (Fab-Fc), the monovalent antibody composed of one H chain, one light chain and one Fc region, the monovalent antibody in that one scFv is fused to a Fc region (scFv-Fc), the monovalent antibody composed of one scFv-Fc and one Fc region, the monovalent antibody composed of a H chain of an antibody and FL fusion polypeptide, wherein the monovalent antibodies can antagonize TL1A induced DR3 activity with decreased or no agonistic activity.

In another more preferable embodiment of the present invention, the antibody fragments include the bivalent antibody in that two Fabs are fused to a IgG2-Fc region [(Fab)₂-IgG2Fc], the bivalent antibody in that two scFvs are fused to a IgG2Fc region [(scFv)₂-IgG2Fc], wherein the bivalent antibodies can antagonize TL1A induced DR3 activity with decreased or no agonistic activity.

In one preferable embodiment, monovalent antibodies of the present invention include the monovalent antibody comprising 6 CDR sequences of the anti-DR3 monoclonal antibody 142A2 or 142S38B, monovalent antibodies comprising amino acid sequences of CDR1 to CDR3 of VH of SEQ ID NOs:16-18 and amino acid sequences of CDR1 to CDR3 of VL of SEQ ID NOs:22-24, and monovalent antibodies comprising amino acid sequences of CDR1 to CDR3 of VH of SEQ ID NOs:28-30 and amino acid sequences of CDR1 to CDR3 of VL of SEQ ID NOs:34-36. In another preferable embodiment, the bivalent IgG2 antibodies of the present invention include the bivalent IgG2 antibody comprising 6 CDR sequences of the anti-DR3 monoclonal antibody 142A2 or 142S38B, bivalent IgG2 antibodies comprising amino acid sequences of CDR1 to CDR3 of VH of SEQ ID NOs:16-18 and amino acid sequences of CDR1 to CDR3 of VL of SEQ ID NOs:22-24, and bivalent IgG2 antibodies comprising amino acid sequences of CDR1 to CDR3 of VH of SEQ ID NOs:28-30 and amino acid sequences of CDR1 to CDR3 of VL of SEQ ID NOs:34-36.

In one preferable embodiment of the present invention, the monovalent antibodies include monovalent antibodies comprising 6 CDR sequences of an anti-DR3 monoclonal antibody 142A2 or 142S38B, monovalent antibodies comprising amino acid sequences of CDR1 to CDR3 of VH of SEQ ID NOs:16-18 and amino acid sequences of CDR1 to CDR3 of VL of SEQ ID NOs:22-24, and monovalent antibodies comprising amino acid sequences of CDR1 to CDR3 of VH of SEQ ID NOs:28-30 and amino acid sequences of CDR1 to CDR3 of VL of SEQ ID NOs:34-36.

In another preferable embodiment of the present invention, the monovalent antibodies also includes monovalent antibodies comprising each 6CDR sequences described below; CDRs of VH of SEQ ID NOs:16, 80, 18 and CDRs of VL of SEQ ID NOs:22-24, CDRs of VH of SEQ ID NOs:16-18 and CDRs of VL of SEQ ID NOs:22, 81, 24, CDRs of VH of SEQ ID NOs:16-18 and CDRs of VL of SEQ ID NOs:22, 82, 24, CDRs of VH of SEQ ID NOs:16-18 and CDRs of VL of SEQ ID NOs:22, 83, 24, CDRs of VH of SEQ ID NOs:16, 80, 18 and CDRs of VL of SEQ ID NOs:22, 81, 24, CDRs of VH of SEQ ID NOs: 16, 80, 18 and CDRs of VL of SEQ ID NOs:22, 82, 24, or CDRs of VH of SEQ ID NOs:16, 80, 18 and CDRs of VL of SEQ ID NOs:22, 83, 24.

In one embodiment, antibodies or antibody fragments of the present invention include an antibody in that any post translational modified amino acid residues are included. An antibody is well known in a post-translational modification like lysine residue is deficient at the C-terminal of a heavy chain (hereinafter, so called as “lysine clipping”) and glutamine residue is modified to pyro-glutamine at the N-terminal of polypeptide (pyroGlu) (Beck et al, Analytical Chemistry, 2013; 85:715-736). Accordingly, in one particular embodiment of the present invention, antibodies comprise pyroGlu and/or Lys clipping at N/C terminal of each polypeptide. These modifications don't essentially influence on an activity of antibodies and antibodies of the present invention have equivalent activities as antibodies without these modifications.

The present invention also provides an antibody composition or antibody fragment composition comprising antibodies or antibody fragments described in the above. In one embodiment, antibody compositions of the present invention include an antibody composition comprising antibodies that neutralize an activity of DR3 and have decreased or no agonistic activity, an antibody composition comprising antibodies that block TL1A binding, neutralize an activity of DR3 and have decreased or no agonistic activity, an antibody composition comprising inhibit the formation of DR3 trimers and neutralize DR3 activation through TL1A binding, wherein the antibodies have a decreased/no agonistic activity for DR3. In one embodiment of the present invention, the antibody composition can comprise variable antibody molecules such as the above post translational modified antibody molecules including pyroGlu and/or Lys clipping at N/C terminal of each polypeptide, glycoform variants, and the like.

### Regulation of a Property of Antibody

In the present invention, an antibody which has a Fc region can exhibit several effector activities through Fc receptor binding or complement binding.

The effector activity refers to an antibody-dependent activity that is mediated by the Fc region of an antibody. As the effector activity, antibody-dependent cellular cytotoxicity activity (ADCC activity), complement-dependent cytotoxicity activity (CDC activity), and antibody-dependent phagocytosis (ADP activity) caused by phagocytes such as macrophages, dendritic cells or the like are known. In the present invention, the ADCC and CDC activities can be measured using known measurement methods [Cancer Immunol. Immunother., 36, 373 (1933)].

The ADCC activity refers to an activity in which an antibody bound to an antigen on a target cell binds to an Fc receptor of an immunocyte via the Fc region of the antibody, thereby activating the immunocyte (a natural killer cell or the like) and damaging the target cell.

The Fc receptor (hereinafter, referred to as FcR in some cases) refers to a receptor binding to the Fc region of an antibody, and induces various types of effector activity due to the binding of an antibody.

FcR corresponds to antibody subclasses, and IgG, IgE, IgA, and IgM specifically bind to FcγR, FcεR, FcαR, and FcμR respectively. FcγR has subtypes including FcγRI(CD64), FcγRII(CD32) and FcγRIII(CD16), and the subtypes respectively have isoforms including FcγRIA, FcγRIB, FcγRIC, FcγRIIA, FcγRIIB, FcγRIIC, FcγRIIIA, FcγRIIIB. These different types of FcγR exist on different cells [Annu Rev. Immunol. 9:457-492 (1991)]. In human beings, FcγRIIIB is specifically expressed in neutrophils, and FcγRIIIA is expressed in monocytes, Natural Killer cells (NK cells), and a portion of T cells. The antibody binding caused via FcγRIIIA induces NK cell-dependent ADCC activity.

The CDC activity refers to an activity in which an antibody bound to an antigen on a target cell activates a series of cascades (complement activation pathways) consisting of a group of complement-related proteins in the blood, thereby damaging the target cell. By the protein fragments generated due to the complement activation, it is possible to induce migration and activation of immunocytes. When C1q having a binding domain for the Fc region of an antibody binds to the Fc region, and C1r and C1s as two serine proteases bind thereto, a C1 complex is formed, whereby the cascade of CDC activity begins.

The method for controlling the effector activity of the antibody of the present invention can be exemplified as follows. Examples of methods of controlling the effector activity of the antibody can include a method of controlling the amount of fucose (also referred to as core fucose) which forms α1, 6-bound to N-acetylglucosamine (GlcNAc) present in a reducing end of a complex type N-linked sugar chain (hereinafter, simply abbreviated to complex sugar chain in some cases) bound to Asn at position 297 of the EU index using the amino acid sequence of Fec of IgG1 subclass as Fc of the antibody of the present invention (WO 2005/035586, WO 2002/31140, WO 00/61739), or a method of controlling the activity by substituting amino acid residues of Fc region of the antibody.

1) Regulation by Modification of Sugar Chains

The effector activity of the antibody can be increased or decreased by controlling the content of fucose that is added to N-acetylglucosamine in the reducing end of the complex sugar chain bound to the Fc region of the antibody.

The method for decreasing the content of fucose binding to the complex-type N-linked sugar chain bound to the Fc region of the antibody is to obtain the antibody with no fucose binding thereto by expressing an antibody using CHO cell from which α1,6-fucosyltransferase gene (FUT8) is deleted. The antibody with no fucose binding thereto has high ADCC activity.

On the other hand, the method for increasing the content of fucose binding to the complex-type N-linked sugar chain bound to the Fc region of the antibody is to obtain the antibody with fucose binding thereto by expressing the antibody using a host cell in which α1,6-fucosyltransferase gene is introduced. The antibody with fucose binding thereto has lower ADCC activity than the antibody with no fucose binding thereto.

In the Fc region of the antibody of the present invention, the N-linked sugar chain is bound to the Asn residue at position 297 of the EU index, but there is no report that sugar chain is bound to the Asn residue of other Fc region. Therefore, two N-glycoside linked sugar chains are typically bound to one molecule of the antibody.

The known N-linked sugar chains are high mannose type, complex type and hybrid type sugar chains. As long as the N-linked sugar chain has no fucose binding thereto, it has higher ADCC activity than the sugar chain with fucose binding thereto.

The complex-type sugar chain bound to the Fc region of the antibody of the present invention can include a sugar chain in which one or more of N-acetylglucosamine (GlcNAc) or galactose-N-acetylglucosamine (hereinafter, referred to as GlcNAc or Gal-GlcNAc) are α1-2- or α1-4-linked to mannose (Man) at the non-reducing end of the core structure (tri-mannosyl core structure). It can also include a complex-type sugar chain having sialic acid, bisecting N-acetylglucosamine (hereinafter, referred to as bisecting GlcNAc), or the like at the non-reducing end of Gal-GlcNAc.

In the present invention, the core-fucose or α1,6-fucose refers to a sugar chain structure in which the 1-position of fucose (hereinafter, referred to as Fuc in some cases) is bound to the 6-position of N-acetylglucosamine (hereinafter, referred to as GlcNAc in some cases) in the reducing end through α-bond of a complex type N-glycoside-linked sugar chain. Further, those having no core fucose bound to N-acetylglucosamine in the reducing end of the complex type N-glycoside-linked sugar chain are simply referred to as sugar chains with no fucose or no core fucose.

In the present invention, the core structure or the tri-mannosyl core structure refers to a Manα1-6(Manα1-3)Manβ1-4GlcNAcβ1-4GlcNAc structure.

As the sugar chain bound to the antibody of the present invention, a biantennary N-glycoside linked complex sugar chain (also called biantennary complex sugar chain) is represented by the following Chemical Formula.

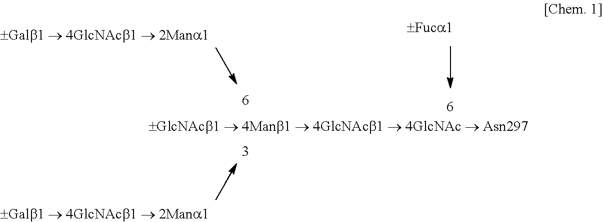

The antibody composition of the present invention is an antibody molecule having the Fc region in which the complex-type sugar chain is bound to Asn at position 297 of the antibody molecule, and as long as it has the above sugar structure, it can be composed of antibody molecules having a single or plural different sugar chains.

In other words, the antibody composition of the present invention means a composition that is composed of antibody molecules having a single or plural different sugar chains, and specifically, a antibody, in which the sugar chain with no fucose bound to N-acetylglucosamine at the reducing end of the sugar chain among the total N-glycoside linked sugar chains bound to the Fc region included in the antibody is 50% or more. In another embodiment of the antibody composition, an antibody composition that is composed of antibody molecules having fucosylated sugar chain in the Fc region is 80% or more.

The ratio of the sugar chain with no core fucose can be any ratio in the antibody composition, as long as ADCC activity of the antibody is increased or decreased. The ratio for increased ADCC can be preferably 50% or more, more preferably 51% to 100%0/, much more preferably 80% to 100%, particularly preferably 90%, 91%, 92%, 93%, 94%, 95%, 96%, 97%, 98%, 99%, and most preferably 100%. The ratio for deceased ADCC can be preferably 20% or less.

The antibody composition having 50% of the ratio of the sugar chain with no core fucose can be any of a antibody composition comprising 100% of molecules with no fucose at one sugar chain of the N-glycoside linked sugar chains bound to the first and second polypeptides of the antibody molecule, and a antibody composition comprising 50% of molecules with no fucose at both sugar chains of the N-glycoside linked sugar chains bound to the first and second polypeptides of the antibody molecule and 50% of molecules with fucose at both sugar chains of the N-glycoside linked sugar chains bound to the first and second polypeptides of the antibody molecule.

In the present invention, the sugar chain with no fucose can have any structure of the sugar chain at the non-reducing end, as long as fucose does not bind to N-acetylglucosamine at the reducing end in the above Chemical Formula.

In the present invention, no fucose (no core fucose) bound to N-acetylglucosamine at the reducing end of the sugar chain means that fucose is not substantially bound. The antibody composition in which “fucose is not substantially bound” means a antibody composition in which fucose cannot be substantially detected in the sugar chain analysis described below. The “fucose cannot be substantially detected” means that it is below the detection limit. The antibody composition with no core fucose in all of the sugar chains has the highest ADCC activity.

The ratio of antibody molecules having sugar chains with no fucose contained in the composition which is composed of a antibody molecule having the Fc region bound with complex-type N-glycoside-linked sugar chains can be determined by releasing the sugar chains from the antibody molecule using a known method such as hydrazinolysis or enzyme digestion [Biochemical Experimentation Methods 23-Method for Studying Glycoprotein Sugar Chain (Japan Scientific Societies Press), edited by Reiko Takahashi (1989)], carrying out fluorescence labeling or radioisotope labeling of the released sugar chains and then separating the labeled sugar chains by chromatography.

Also, the ratio of antibody molecules bound with sugar chains with no fucose contained in the composition which is composed of a antibody molecule having the Fc region bound with complex-type sugar chains can be determined by analyzing the released sugar chains with the HPAED-PAD method [J. Liq. Chromatogr., 6, 1577 (1983)].

2) Regulation by Substitution of Amino Acid Residues

The ADCC, ADCP, and CDC activities of the antibody or the antibody fragment of the present invention can be increased or decreased by changing antibody subclass of Fc constituting the antibody or by substituting the amino acid residues of Fc region.

For example, CDC activity of the antibody can be increased by using the amino acid sequence of the Fe region, which is described in US Patent Application Publication No. 2007/0148165. Also, ADCC activity or CDC activity of the antibody can be increased or decreased by carrying out substitution of the amino acid residues, which is described in U.S. Pat. Nos. 6,737,056, 7,297,775, and 7,317,091.

Specific substitution for increasing ADCC activity can include S239D, P247I, F243L, R292P, S298A, Y300L, A330L, 1332E, E333A, K334A, A339D, T393A, P396L, H433P, or the like. Meanwhile, specific substitution for reducing ADCC activity can include L235E, P238A, N297A, K322A, P331S or the like.

CDC activity can be increased in combinations of two or more of amino acid residue substitutions, and amino acid residues to be substituted can be increased depending on the purpose. Preferably, the amino acid residue substitution for increasing CDC activity can include at least one substitution selected from N276K, A339T, T394F and T394Y, amino acid residue substitutions of N276K and A339T, and amino acid residue substitutions of K274Q, N276K, Y296F, Y300F, A339T, D356E, L358M, N384S, V397M and V422I, or the like. Meanwhile, specific amino acid residue substitution for reducing CDC activity can include at least one substitution selected from L235E, N297A, K322A, P329A and P331S or the like.

The blood half-life can be also prolonged by adding at least one substitution selected from T250Q, M428L, M252Y, S254T, T256E, or the like, into Fc of human IgG subclass. Cell cytotoxicity such as ADCC activity, ADCP activity, CDC activity can be also reduced by using Fc in which N-linked sugar chain is removed by introduction of amino acid mutation at position N297, Fc of human IgG2 or IgG4 subclass, chimeric Fc of IgG2 and IgG4, or the like.

In one embodiment of the present invention, antibodies or antibody fragments can be stabilized in low pH condition, by adding at least a substitution selected from F300Y, V309L and T339A into human IgG antibody.

In a preferable embodiment, antibodies or antibody fragments of the present invention comprise at least one amino acid substitution selected from 234, 235, 237, 250, 300, 309, 331, 339, 409 and 428 positions in the Fc region of IgG antibody. In more preferable embodiment, antibodies of the present invention include an antibody comprising at least one amino acid substitution selected from V234A, L235E, G237A, T250Q, F300Y, V309L, P331S, T339A and M428L in the Fc region of human IgG2 antibody, an antibody comprising amino acid substitutions of T250Q and M428L in the Fc region of human IgG2 antibody, an antibody comprising amino acid substitutions of V234A, G237A and P331S in the Fc region of human IgG2 antibody, an antibody comprising amino acid substitutions of F300Y, V309L and T339A in the Fc region of human IgG2 antibody, and an antibody comprising amino acid substitutions of V234A, L235E, G237A, T250Q, F300Y, V309L, T339A, P331S and M428L in the Fc region of human IgG2 antibody or the like.

In one embodiment, antibody fragments of the present invention comprise as following;

(i) at least one amino acid substitution selected from 214, 220, 435 and 436 positions in the Fc region of IgG1 antibody.
(ii) at least one amino acid substitution selected from 234, 235, 237, 250, 300, 309, 339, 331, 409, 428, 435 and 436 positions in the Fc region of IgG2 antibody, or
(iii) at least one amino acid substitution selected from 131, 133, 228, 235, 409, 435 and 436 positions in the Fc region of IgG4 antibody

In one preferable embodiment, antibody fragments of the present invention include the antibody fragment comprising as following;

(i) at least one amino acid substitution selected from C214S, C220S, H435R and Y436F in the Fc region of IgG1 antibody.
(ii) at least one amino acid substitution selected from C220S, V234A, L235E, G237A, T250Q, F300Y, V309L, P331S, T339A, M428L, H435Y and Y436F positions in the Fc region of IgG2 antibody, or
(iii) at least one amino acid substitution selected from C131S, R133K, C214S, S228P, L235E, R409K, H435R and Y436F in the Fc region of IgG4 antibody

In the most preferable embodiment, monovalent antibodies of the present invention include as following;

(i) a monovalent antibody comprising C220S substitution in a H chain and C214S, H435R, and Y436F substitutions in FL chain of IgG1,
(ii) a monovalent antibody comprising C220S substitution in a H chain, C214S, H435R and Y436F substitutions and a deletion of EPKSC of 216-220 in FL chain of IgG1,
(iii) a monovalent antibody comprising C220S, L235E, G237A and P331S substitutions in a H chain and C214S, C220S, L235E, G237A, P331S. H435R, and Y436F substitutions in FL chain of IgG1,
(iv) a monovalent antibody comprising C220S, L235E, G237A and P331S substitutions in a H chain, C214S, L235E, G237A, P331S, H435R, and Y436F substitutions and a deletion of EPKSC of 216-220 in FL chain of IgG1,
(v) a monovalent antibody comprising C220S and F300Y substitutions in a H chain, C214S, F300Y, H435R, and Y436F in FL chain of IgG2,
(vi) a monovalent antibody comprising C220S, T250Q and M428L substitutions in a H chain, C214S, T250Q, M428L, H435R, and Y436F in FL chain of IgG2,
(vii) a monovalent antibody comprising C220S, V234A, L235E, G237A, T250Q, F300Y, V309L, P331S, T339A and M428L substitution in a H chain, C214S, V234A, L235E, G237A, T250Q, F300Y, V309L, T339A, P331S, M428L, H435R, and Y436F in FL chain of IgG2,
(viii) a monovalent antibody comprising C220S, V234A, L235E, G237A, T250Q, F300Y, V309L, P331S, T339A and M428L substitution in a H chain, C214S, V234A, L235E, G237A, T250Q, F300Y, V309L, T339A, P331S, M428L, H435R, and Y436F in FL chain of IgG2 and
(ix) a monovalent antibody comprising C131S and R409K substitutions in a H chain and C214S, R409K, H435R and Y436F substitutions in FL chain of IgG4.
(x) a monovalent antibody comprising C131S, R133K, S228P, L235E and R409K substitutions in a H chain and C214S, S228P, L235E, R409K, H435R and Y436F substitutions in FL chain of IgG4.

The present invention also includes a method of producing antibodies and antibody fragment thereof of the present invention comprising a step a) vectors comprising a nucleic acid sequence encoding amino acid sequences of the antibody are introduced into a host cell, b) culturing the cell and recovering a culture supernatant, and c) purifying the antibody from the culture supernatant.

In preferable embodiments of the present invention, the method includes:

a method of producing an antibody comprising steps a) replacing to a part of a CH1 domain and/or a hinge domain derived from IgG2 subclass in a constant region of an antibody, b) vectors comprising a nucleic acid sequence encoding amino acid sequences of an antibody are introduced into a host cell, c) culturing the cell and recovering a culture supematant, and d) purifying the antibody from the culture supernatant,

a method of producing an antibody comprising steps a) replacing to IgG2 subclass in a Fc region of an antibody, b) vectors comprising a nucleic acid sequence encoding amino acid sequences of an antibody are introduced into a host cell, c) culturing the cell and recovering a culture supernatant, and d) purifying the antibody from the culture supernatant,

a method producing an IgG2 antibody or an IgG2 antibody variant comprising a step a) vectors comprising a nucleic acid sequence encoding amino acid sequences of the antibody are introduced into a host cell, b) culturing the cell and recovering a culture supernatant, and c) purifying the antibody from the culture supernatant,

a method of producing an IgG2 antibody or an IgG2 antibody variant comprising a step a) vectors comprising a nucleic acid sequence encoding amino acid sequences of the antibody are introduced into a host cell, b) culturing the cell and recovering a culture supernatant, and c) purifying the antibody from the culture supernatant, and

a method of producing an IgG2 antibody or an IgG2 antibody variant comprising a step a) vectors comprising a nucleic acid sequence encoding amino acid sequences of the antibody comprising at least one amino acid substitution selected from 234, 235, 237, 250, 300, 309, 331, 339, 409, 428, 435 and 436 positions in the Fc region of IgG antibody, or IgG2 antibody variant comprising a hinge domain of IgG2 antibody and CH1, CH2, CH3 domains of IgG4, wherein an amino acid residue is Lys at position 409 in the CH3 domain, are introduced into a host cell, b) culturing the cell and recovering a culture supernatant, and c) purifying the antibody from the culture supernatant.

The present invention also includes:

a method of producing antibody fragments of the present invention comprising a step a) a vector encoding amino acid sequences of an antibody fragment of IgG1, IgG2 or IgG4 antibody, is introduced into a host cell, b) culturing the cell and recovering a culture supernatant, and c) purifying the antibody from the culture supernatant,

a method of producing antibody fragments of the present invention comprising a step a) a vector encoding amino acid sequences of an antibody fragment comprising Fc region of IgG1, IgG2 or IgG4 antibody, is introduced into a host cell, b) culturing the cell and recovering a culture supernatant, and c) purifying the antibody from the culture supernatant and

a method of producing antibody fragments of the present invention comprising a step a) a vector encoding amino acid sequences of a monovalent antibody comprising Fc region of IgG1, IgG2 or IgG4 antibody, is introduced into a host cell, b) culturing the cell and recovering a culture supernatant, and c) purifying the antibody from the culture supernatant. Further any method of producing antibody fragments in the above described can be included in the method of the present invention.

In one embodiment of the present invention, the method includes:

a method of decreasing or deleting an agonism of an antibody comprising step a) replacing to a part of a CH1 domain and/or a hinge domain derived from IgG2 subclass in a constant region of an antibody, b) producing a recombinant antibody and c) detecting an agonistic activity of the antibody, and

a method of decreasing or deleting an agonism of an antibody comprising step a) replacing to IgG2 subclass or IgG2 antibody variant in a Fc region of an antibody, b) producing a recombinant antibody and c) detecting an agonistic activity of the antibody.

In preferable embodiment, the method of the invention includes a method of decreasing or deleting an agonism of an antibody comprising step a) replacing to IgG2 subclass or IgG2 antibody variant in an Fc region of an antibody, b) producing a recombinant antibody and c) detecting an agonistic activity of the antibody in at least one assay selected from (i) phosphorylation of DR3, (ii) phosphorylation of NF-κB, (iii) the proliferation of T cells, (iv) apoptosis of DR3 positive cells and (v) cytokine releases from T cells such as interleukin (IL)-13, IL-17, GM-CSF and IFN-γ.

In one embodiment of the present invention, the method includes:

a method of decreasing or deleting an agonism of an antibody fragment comprising step a) constructing a vector encoding amino acid sequences of an antibody fragment of IgG1, IgG2, IgG4 or each antibody variant, b) producing a recombinant antibody and c) detecting an agonistic activity of the antibody,

a method of decreasing or deleting an agonism of an antibody fragment comprising a) constructing a vector encoding amino acid sequences of an antibody fragment comprising Fc region of IgG1, IgG2 or IgG4 antibody, b) producing a recombinant antibody and c) detecting an agonistic activity of the antibody, and

a method of decreasing or deleting an agonism of an antibody fragment comprising a) constructing a vector encoding amino acid sequences of a monovalent antibody comprising Fc region of IgG1, IgG2, IgG4 or each antibody variant, b) producing a recombinant antibody and c) detecting an agonistic activity of the antibody.

In preferable embodiment, the method of the invention includes a method of decreasing or deleting an agonism of an antibody comprising step a) reconstructing a monovalent antibody comprising a Fc region of IgG1, IgG2, IgG4 or each antibody variant, b) producing a recombinant monovalent antibody and c) detecting an agonistic activity of the monovalent antibody in at least one assay selected from (i) phosphorylation of DR3, (ii) phosphorylation of NF-κB, (iii) the proliferation of T cells, (iv) apoptosis of DR3 positive cells and (v) cytokine releases from T cells such as interleukin (IL)-13, IL-17, GM-CSF and IFN-γ.

A process for producing the antibody of the present invention, a method for treating the disease and a method for diagnosing the disease are specifically described below.

### 1. Preparation Method of Monoclonal Antibody

### (1) Preparation of Antigen

DR3 polypeptide or a cell expressing DR3 protein as an antigen can be obtained by introducing an expression vector comprising cDNA encoding a full length of DR3 or a partial length thereof is introduced into Escherichia coli, yeast, an insect cell, an animal cell or the like. In addition, DR3 can be purified from various human tumor cell lines, human tissue and the like which express a large amount of DR3. The tumor cell line and the tissue can be allowed to use as antigens. Furthermore, a synthetic peptide having a partial sequence of the DR3 can be prepared by a chemical synthesis method such as Fmoc method or tBoc method and used as an antigen. Further any species of DR3 protein such as human, monkey, rat, mouse and the like, are expressed and prepared.

DR3 used in the present invention can be produced, for example, by expressing a DNA encoding DR3 in a host cell using a method described in Molecular Cloning, A Laboratory Manual, Second Edition, Cold Spring Harbor Laboratory Press (1989), Current Protocols in Molecular Biology, John Wiley & Sons (1987-1997) or the like according to the following method.

Firstly, a recombinant vector is prepared by inserting a full length cDNA comprising the region encoding DR3 or a fragment thereof into downstream of a promoter of an appropriate expression vector. At this time, if necessary, a DNA fragment having an appropriate length containing a region encoding the polypeptide based on the full length cDNA, and the DNA fragment may be used instead of the above full length cDNA. Next, a transformant producing DR3 can be obtained by introducing the recombinant vector into a host cell suitable for the expression vector.

The expression vector includes vectors which can replicate autonomously in the host cell to be used or vectors which can be integrated into a chromosome comprising an appropriate promoter at such a position that the DNA encoding the polypeptide can be transcribed.

The host cell may be any one, so long as it can express the objective gene. Examples include a microorganism which belongs to the genera Escherichia, such as Escherichia coli, yeast, an insect cell, an animal cell and the like.

When a prokaryote such as Escherichia coli is used as the host cell, it is preferred that the recombinant vector used in the present invention is autonomously replicable in the prokaryote and comprises a promoter, a ribosome binding sequence, the DNA comprising the portion encoding DR3 and a transcription termination sequence. The recombinant vector is not necessary to have a transcription termination sequence, but a transcription termination sequence is preferably set just below the structural gene. The recombinant vector may further comprise a gene regulating the promoter.

Also, the above recombinant vector is preferably a plasmid in which the space between Shine-Dalgarno sequence (also referred to as SD sequence), which is the ribosome binding sequence, and the initiation codon is adjusted to an appropriate distance (for example, 6 to 18 nucleotides).

Furthermore, the nucleotide sequence of the DNA encoding DR3 can be substituted with another base so as to be a suitable codon for expressing in a host cell, thereby improve the productivity of the objective DR3.

Any expression vector can be used, so long as it can function in the host cell to be used. Examples of the expression vector includes pBTrp2, pBTac1, pBTac2 (all manufactured by Roche Diagnostics), pKK233-2 (manufactured by Pharmacia), pSE280 (manufactured by Invitrogen), pGEMEX-1 (manufactured by Promega), pQE-8 (manufactured by QIAGEN), pKYP10 (Japanese Published Unexamined Patent Application No. 110600/83), pKYP200 [Agricultural Biological Chemistry, 48, 669 (1984)], pLSA1 [Agric. Biol. Chem., 53, 277 (1989)], pGEL1 [Proc. Natl. Acad. Sci. USA, 82, 4306 (1985)], pBluescript II SK(−) (manufactured by Stratagene), pTrs30 [prepared from Escherichia coli JM109/pTrS30 (FERM BP-5407)], pTrs32 [prepared from Escherichia coli JM109/pTrS32 (FERM BP-5408)], pGHA2 [prepared from Escherichia coli IGHA2 (FERM BP-400), Japanese Published Unexamined Patent Application No. 221091/85], pGKA2 [prepared from Escherichia coli IGKA2 (FERM BP-6798), Japanese Published Unexamined Patent Application No. 221091/85], pTerm2 (U.S. Pat. No. 4,686,191, U.S. Pat. No. 4,939,094, U.S. Pat. No. 5,160,735), pSupex, pUB110, pTP5, pC194, pEG400 [J. Bacteriol., 172, 2392 (1990)], pGEX (manufactured by Pharmacia), pET system (manufactured by Novagen), pME18SFL, and the like.

Any promoter can be used, so long as it can function in the host cell to be used. Examples include promoters derived from Escherichia coli, phage and the like, such as trp promoter (Ptrp), lac promoter, PL promoter, PR promoter and T7 promoter. Also, artificially designed and modified promoters, such as a promoter in which two Ptrp are linked in tandem, tac promoter, lacT7 promoter and letI promoter, can be used.

Examples of the host cell include Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli DH5a and the like.

Any introduction method of the recombinant vector can be used, so long as it is a method for introducing DNA into the host cell, and examples include a method using a calcium ion [Proc. Natl. Acad. Sci. USA, 69, 2110 (1972), methods described in Gene, 17, 107 (1982) and Molecular & General Genetics, 168, 111 (1979)].

When an animal cell is used as a host, any expression vector may can be used as long as it exhibits its functions in animal cells, and examples thereof may can include pcDNAI, pCDM8 (manufactured by Funakoshi co.), pAGE107 [Japanese Patent Publication No. H3-22979; Cytotechnology, 3, 133 (1990)], pAS3-3 (Japanese Patent Publication No. H2-227075), pcDNAI/Amp (manufactured by Invitrogen), pcDNA 3.1 (manufactured by Invitrogen), pREP4 (manufactured by Invitrogen), pAGE103 [J. Biochemistry, 101, 1307 (1987)], pAGE210, pME18SFL3, pKANTEX93 (WO 97/10354), N5KG1val (U.S. Pat. No. 6,001,358), Tol2 transposon vector (WO 2010/143698, WO02013/005649) or the like.

Any promoter can be used, so long as it can function in an animal cell. Examples include a promoter of immediate early (IE) gene of cytomegalovirus (CMV), SV40 early promoter, a promoter of retrovirus, a metallothionein promoter, a heat shock promoter, SR α promoter, Molony murine leukemia virus promoter or enhancer, and the like. Also, the enhancer of the IE gene of human CMV can be used together with the promoter. Examples of the host cell include human leukemia cell Namalwa, monkey COS cell, human leukemia cell PER.C6, Chinese hamster ovary cell CHO cell (Journal of Experimental Medicine, 108, 945 (1958); Proc. Natl. Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Natl. Acad. Sci. USA, 77, 4216 (1980); Proc. Natl. Acad. Sci., 60, 1275 (1968); Cell, 6, 121 (1975); Molecular Cell Genetics, Appendix I, II (pp. 883-900)), CHO/DG44, CHO-K1 (ATCC CCL-61), DUkXB11 (ATCC CCL-9096), Pro-5 (ATCC CCL-1781), CHO-S(Life Technologies, Cat no. 11619), Pro-3 cell, rat myeloma YB2/3HL.P2.G11.16Ag.20 (also known as YB2/0), mouse myeloma cell NSO, mouse myeloma cell SP2/0-Ag14, Syrian hamster cells BHK or HBT5637 (Japanese Published Unexamined Patent Application No. 000299/88) and the like.

Any introduction method of the recombinant vector can be used, so long as it is a method for introducing DNA into an animal cell, and examples include electroporation [Cytotechnology, 3, 133 (1990)], the calcium phosphate method (Japanese Published Unexamined Patent Application No. 227075/90), the lipofection method [Proc. Natl. Acad. Sci. USA, 84, 7413 (1987)], and the like.

DR3 can be produced by culturing the transformant derived from a microorganism, an animal cell or the like having a recombinant vector comprising the DNA encoding DR3 in a medium to form and accumulate DR3 in the culture, and recovering it from the culture. The method for culturing the transformant in the medium is carried out according to the usual method used in culturing of hosts. When DR3 is expressed in a cell derived from eukaryote, DR3 bound to sugars or sugar chains can be obtained. When a microorganism transformed with a recombinant vector containing an inducible promoter is cultured, an inducer can be added to the medium, if necessary. For example, isopropyl-β-D-thiogalactopyranoside or the like can be added to the medium when a microorganism transformed with a recombinant vector using lac promoter is cultured; or indoleacrylic acid or the like can be added thereto when a microorganism transformed with a recombinant vector using trp promoter is cultured.

When a transformant obtained using an animal cell as the host cell is cultured, the medium includes generally used RPMI 1640 medium [The Journal of the American Medical Association, 199, 519 (1967)], Eagle's MEM medium [Science, 122, 501 (1952)], Dulbecco's modified MEM medium [Virology, 8, 396 (1959)] and 199 medium [Proceeding of the Society for the Biological Medicine, 73, 1 (1950)], Iscoove's modified Dulbecco's medium (IMDM), the media to which fetal calf serum, etc. is added, and the like. The culturing is carried out generally at a pH of 6 to 8 and 30 to 40° C. for 1 to 7 days in the presence of 5% CO₂. If necessary, an antibiotic such as kanamycin, penicillin or gentamicine can be added to the medium during the culturing.

Regarding the expression method of the gene encoding DR3, in addition to direct expression, secretory production, fusion protein expression and the like can be carried out according to the method described in Molecular Cloning, A Laboratory Manual, Second Edition, Cold Spring Harbor Laboratory Press (1989). In one embodiment, DR3 fusion protein includes a DR3 Fc fusion protein, an extracellular region of DR3 Fc fusion protein, a CRD domain Fc fusion protein, DR3 histidine tag (abbreviated as His-tag) fusion protein, DR3 Flag fusion protein and the like. In the any event, any DR3, DR3 variants and a fragment thereof can be fused to Fc, His-tag, Flag-tag, glutathione-S transferase (GST)-tag, or the like, and be produced.

The process for producing DR3 includes a method of intracellular expression in a host cell, a method of extracellular secretion from a host cell, a method of producing on a host cell membrane outer envelope, and the like. The appropriate method can be selected by changing the host cell used and the structure of the DR3 produced.

When the DR3 is produced in a host cell or on a host cell membrane outer envelope, DR3 can be positively secreted extracellularly in accordance with the method of Paulson et al. [J. Biol. Chem., 264, 17619 (1989)], the method of Lowe et al. [Proc. Natl. Acad. Sci. USA, 86, 8227 (1989), Genes Develop., 4, 1288 (1990)], the methods described in Japanese Published Unexamined Patent Application No. 336963/93 and WO 94/23021, and the like. Also, the production amount of DR3 can be increased in accordance with the method described in Japanese Published Unexamined Patent Application No. 227075/90 utilizing a gene amplification system using a dihydrofolate reductase (DHFR) gene.

The resulting DR3 can be isolated and purified, for example, as follows. When DR3 is intracellularly expressed in a dissolved state, the cells after culturing are recovered by centrifugation, suspended in an aqueous buffer and then disrupted using ultrasonicator, French press, Manton Gaulin homogenizer, dynomill or the like to obtain a cell-free extract. The cell-free extract is centrifuged to obtain a supematant, and a purified preparation can be obtained by subjecting the supernatant to a general enzyme isolation and purification techniques such as solvent extraction; salting out with ammonium sulfate etc.; desalting; precipitation with an organic solvent; anion exchange chromatography using a resin such as diethylaminoethyl (DEAE)-sepharose, DIAION HPA-75 (manufactured by Mitsubishi Chemical); cation exchange chromatography using a resin such as S-Sepharose FF (manufactured by Pharmacia); hydrophobic chromatography using a resin such as butyl-Sepharose or phenyl-Sepharose; gel filtration using a molecular sieve; affinity chromatography; chromatofocusing; electrophoresis such as isoelectric focusing; and the like which may be used alone or in combination.

When DR3 is expressed intracellularly by forming an inclusion body, the cells are recovered, disrupted and centrifuged in the same manner, and the inclusion body of DR3 are recovered as a precipitation fraction. The recovered inclusion body of the protein is solubilized with a protein denaturing agent. The protein is made into a normal three-dimensional structure by diluting or dialyzing the solubilized solution, and then a purified preparation of DR3 is obtained by the same isolation purification method as above.

When DR3, its variants or the fragment thereof such as a glycosylated product is secreted extracellularly, DR3 or the derivative such as a glycosylated product can be recovered from the culture supernatant. That is, the culture is treated by a method such as centrifugation in the same manner as above to obtain a culture supernatant, a purified preparation of DR3 can be obtained from the culture supernatant by the same isolation purification method as above.

Also, DR3 used in the present invention can be produced by a chemical synthesis method, such as Fmoc method or tBoc method. Also, it can be chemically synthesized using a peptide synthesizer manufactured by Advanced ChemTech, Perkin-Elmer, Pharmacia, Protein Technology Instrument, Synthecell-Vega, PerSeptive, Shimadzu Corporation, or the like.

### (2) Immunization of Animal and Preparation of Antibody-Producing Cell for Fusion

A mouse, rat or hamster 3 to 20 weeks old is immunized with the antigen prepared in the above (1), and antibody-producing cells are collected from the spleen, lymph node or peripheral blood of the animal. Also, when the increase of a sufficient titer in the above animal is recognized due to low immunogenicity, a DR3 knockout mouse may by used as an animal to be immunized.

The immunization is carried out by administering the antigen to the animal through subcutaneous, intravenous, intraperitoneal or intra lympho nodal injection together with an appropriate adjuvant (for example, complete Freund's adjuvant, combination of aluminum hydroxide gel with pertussis vaccine, or the like). When the antigen is a partial peptide, a conjugate is produced with a carrier protein such as BSA (bovine serum albumin), KLH (keyhole limpet hemocyanin) or the like, which is used as the antigen.

The administration of the antigen is carried out 5 to 10 times every one week or every two weeks after the first administration. On the 3rd to 7th day after each administration, a blood sample is collected from the fundus of the eye or tail vein, the reactivity of the serum with the antigen is tested, for example, by enzyme immunoassay [Antibodies-A Laboratory Manual, Cold Spring Harbor Laboratory (1988)] or the like. An animal showing a sufficient antibody titer in their sera against the antigen used for the immunization is used as the supply source of antibody-producing cells for fusion.

Three to seven days after final administration of the antigen, tissue containing the antibody-producing cells such as the spleen, or lympho nodes from the immunized animal is excised to collect the antibody-producing cells. When the spleen cells/lympho nodal cells are used, the tissue is excided out and loosened, followed by centrifuged. Then, antibody-producing cells for fusion are obtained by removing erythrocytes.

### (3) Preparation of Myeloma Cell

An established cell line obtained from mouse is used as myeloma cells. Examples include 8 azaguanine-resistant mouse (derived from BALB/c) myeloma cell line P3-X63Ag8-U1 (P3-U1) [Current Topics in Microbiology and Immunology, 18, 1 (1978)], P3-NS1/1-Ag41 (NS-1) [European J. Immunology, 6, 511 (1976)], SP2/0-Ag14 (SP-2) [Nature, 276, 269 (1978)], P3-X63-Ag8653 (653) [J. Immunology, 123, 1548 (1979)], P3-X63-Ag8 (X63) [Nature, 256, 495 (1975)] and the like.

These cells lines are subcultured in an appropriate medium such as a 8-azaguanine medium [a medium obtained by adding 8-azaguanine to an RPMI-1640 medium supplemented with glutamine, 2-mercaptoethanol, gentamycin, and fetal calf serum (hereinbelow, referred to as “FCS”)], the Iscove's Modified Dulbecco's Medium (hereinafter, referred to as “IMDM”), or the Dulbecco's Modified Eagle Medium (hereinafter, referred to as “DMEM”). The cells are subcultured 3 to 4 days before cell fusion in a normal medium (for example, a DMEM containing 10% FCS) to secure cells in number of 2×10⁷ or more on the day of fusion.

### (4) Cell Fusion and Preparation of Hybridoma for Producing Monoclonal Antibody

The antibody-producing cells for fusion obtained by the above (2) and myeloma cells obtained by the above (3) were sufficiently washed with a minimum essential medium (MEM) or PBS (1.83 g of disodium hydrogen phosphate, 0.21 g of potassium dihydrogen phosphate, 7.65 g of sodium chloride, 1 liter of distilled water, pH 7.2) and mixed to give a ratio of the antibody-producing cells:the myeloma cells=5 to 10:1, followed by centrifugation. Then, the supematant is discarded. The precipitated cell group is sufficiently loosened. After loosening the precipitated cell, the mixture of polyethylene glycol-1000 (PEG-1000), MEM and dimethylsulfoxide is added to the cell under stirring at 37° C. In addition, 1 to 2 mL of MEM medium is added several times every one or two minutes, and MEM is added to give a total amount of 50 mL. After centrifugation, the supernatant is discarded. After the cells are gently loosen, the cells are gently suspended in HAT medium [a medium in which hypoxanthine, thymidine and aminopterin is added to the normal medium]. The suspension is cultured in a 5% CO₂ incubator for 7 to 14 days at 37° C.

When the myeloma cells described above are a 8-azaguanine-resistant line, that is, a hypoxanthine/guanine/phosphoribosyltransferase (HGPRT)-deficient line, the myeloma cells not fused and the fusion cells of the myeloma cells itself can't survive in the HAT-containing medium. On the other hand, the fusion cells of the antibody-producing cells each other and the hybridoma of the antibody-producing cells and the myeloma cells can survive, but life of the fusion cells of the antibody-producing cells is limited. Accordingly, if these cells are continuously cultured in the HAT-containing medium, only the hybridoma of the antibody-producing cells and the myeloma cells can survive, and as a result, it is possible to select the hybridoma.

The medium of the hybridoma grown in a colony shape is replaced with a medium (hereinbelow, referred to as an “HT medium”) obtained by removing aminopterin from the HAT medium. Thereafter, a portion of the supernatant is collected, and then an antibody-producing hybridoma can be selected using the antibody titer measurement method described later.

Examples of the method of measuring antibody titer include various known techniques such as radioimmunoassay (hereinbelow, referred to as an “RIA”), enzyme-linked immunosorbent assay (hereinbelow, referred to as an “ELISA”), a fluorescent antibody method such as flow cytometry, and passive hemagglutination. Among these, in view of detection sensitivity, rapidity, accuracy, possibility of operation automation, and the like, the RIA or ELISA is preferable.

The hybridoma which is confirmed to produce specific antibodies by the antibody titer measurement is transferred to another plate and cloned. Examples of the cloning method include limiting dilution method in which the hybridoma is cultured by being diluted such that one hybridoma is contained in each well of the plate, a soft agar method in which the hybridoma is cultured in a soft agar medium to recover the colony, a method of taking out cells one by one by using a micromanipulator and culturing the cells, and “sorter cloning” in which a single cell is separated by a cell sorter, and the like. Limiting dilution method is widely used due to its simplicity.

Cloning is repeated 2 to 4 times by, for example, limiting dilution for the wells in which the antibody titer is confirmed, and a hybridoma in which the antibody titer is stably confirmed is selected as an anti-human DR3 monoclonal antibody-producing hybridoma line.

### (5) Preparation of Purified Monoclonal Antibody

The hybridoma cells producing a monoclonal antibody obtained by the above (4) are administered by intraperitoneal injection into 8- to 10-week-old mice or nude mice treated with 0.5 mL of pristane (2,6,10,14-tetramethylpentadecane (pristane) is intraperitoneally administered, followed by feeding for 2 weeks). The hybridoma develops ascites tumor in 10 to 21 days. The ascitic fluid is collected from the mice, centrifuged to remove solids, subjected to salting out with 40 to 50% ammonium sulfate and then precipitated by caprylic acid, passed through a DEAE-Sepharose column, a protein A column or a gel filtration column to collect an IgG or IgM fraction as a purified monoclonal antibody.

Furthermore, a monoclonal antibody-producing hybridoma obtained by the above (4) is cultured in RPMI1640 medium containing FBS or the like and the supernatant is removed by centrifugation. The precipitated cells are suspended in Hybridoma SFM medium containing 5% DIGO GF21 and cultured for 3 to 7 days. The purified monoclonal antibody can be obtained by centrifusing the obtained cell suspension, followed by purifying the resulting supernatant with Protein A column or Protein G column to collect the IgG fractions.

The subclass of the antibody can be determined using a subclass typing kit by enzyme immunoassay. The amount of the protein can be determined by the Lowry method or from the absorbance at 280 nm [1.4 (OD₂₈₀)=immunoglobulin 1 mg/mL].

### (6) Selection of Monoclonal Antibody

Binding activity of the anti-DR3 monoclonal antibody of the present invention can be confirmed by a binding assay system such as the Ouchterlony method, ELISA, RIA, a flow cytometry (FCM), or a surface plasmon resonance (SPR) method. Though simple, the Ouchterlony method requires concentration operation when antibody concentration is low.

When the ELISA or RIA is used, the culture supernatant is bound with an antigen-adsorbed solid phase as is, and an antibody corresponding to various immunoglobulin isotypes and subclasses is used as a second antibody, whereby the isotype and subclass of the antibody can be identified.

The purified or partially purified recombinant human DR3 is adsorbed onto a solid phase surface of a 96-well plate for ELISA or the like, and a solid phase surface onto which an antigen is not adsorbed is blocked with a protein irrelevant with an antigen, such as bovine serum albumin (hereinafter, described as “BSA”).

The ELISA plate is washed with phosphate buffer saline (hereinafter, described as “PBS”) containing 0.05% Tween 20 (hereinafter, abbreviated to Tween-PBS) or the like and then bound with a serially diluted first antibody (for example, mouse serum, culture supernatant, or the like), thereby binding the antibody to the antigen immobilized onto the plate.

Thereafter, as a second antibody, an anti-immunoglobulin antibody labeled with biotin, an enzyme (horse radish peroxidase; HRP, alkaline phosphatase; ALP, or the like), a chemiluminescent substance, a radioactive compound, or the like is dispensed to the plate, thereby reacting the second antibody with the first antibody having bound to the plate. After the plate is sufficiently washed with Tween-PBS, a reaction caused by the labeling substance of the second antibody is performed, thereby selecting a monoclonal antibody binding specifically with the immunogen.

Binding activity of a target antibody with respect to an antigen-expressing cell can be measured by the FCM [Cancer Immunol. Immunother., 36, 373 (1993)]. If a target antibody binds to a membrane protein expressed on a cell membrane, this can be mentioned that the target antibody is an antibody which recognizes the three-dimensional structure of a naturally occurring antigen.

Examples of the SPR include kinetics analysis using Biacore®. For example, by using Biacore®T100, kinetics in binding of an antigen to a subject substance is measured, and the resultant thereof is analyzed by analysis software attached to the instrument. After the anti-mouse IgG antibody is immobilized onto a sensor chip CM5 by an amine coupling method, a subject substance such as hybridoma culture supernatant or a purified monoclonal antibody is allowed to flow such that an appropriate amount of the substance binds to the antibody, and then an antigen of different levels of known concentration is allowed to flow, thereby measuring binding and dissociation. The kinetics analysis is performed on the obtained data by using software attached to the instrument by a 1:1 binding model, thereby obtaining various parameters. Alternatively, the human DR3 protein is immobilized onto a sensor chip by, for example, the amine coupling method, and then a purified monoclonal antibody with different levels of known concentration is allowed to flow, thereby measuring binding and dissociation. The kinetic analysis is performed on the obtained data by using software attached to the instrument by a bivalent binding model, thereby obtaining various parameters.

The antibody which competes with the anti-DR3 antibody of the present invention to bind to DR3 can be obtained by adding a subject antibody to the above binding assay system and binding the antibody. That is, by screening an antibody of which the binding activity is inhibited when the subject antibody is added, it is possible to obtain an antibody which competes with the obtained antibody to bind to the extracellular domain of DR3.

### (7) Identification of Epitope of Anti-DR3 Monoclonal Antibody

In the present invention, a recognition epitope of an antibody can be identified in the following manner. For example, a partially deficient antigen, an amino acid substituted antigen obtained by amino acid substitution using different heterogeneous amino acid residues, or a modified antigen obtained by modifying domains is prepared, and when the reactivity of the target antibody with respect to the deficient antigen or the amino acid-substituted antigen is lowered, this clearly shows that the deficient site and the amino acid-substituted site is the epitope recognized by the target antibody. The partially deficient antigen or the amino acid-substituted antigen can be obtained as a protein secreted from an appropriate host cell (Escherichia coli, yeast, a plant cell, a mammal cell such as Chinese Hamster Ovary Cell, or the like). It is also possible to prepare an antigen-expressing cell by expressing the antigen on host cell surface. In a case of a membrane-type antigen, it is preferable to express the antigen on host cell surface so as to express the antigen while retains the conformational structure of the antigen (seems to be a naturally occurring conformation). It is also possible to confirm the reactivity of the target antibody by preparing a synthetic peptide which mimics the primary structure or three-dimensional structure of the antigen. Examples of methods of preparing the synthetic peptide include a method of preparing partial peptides having various molecules by using a known peptide synthesis technique.

Regarding the anti-DR3 antibody of the present invention, chimeric proteins obtained by combining the respective PLAD domain, CRD domains 1 to 4 of the extracellular domain of the human and mouse DR3 are prepared so as to confirm the reactivity of the target antibody, whereby the epitope of the antibody can be identified. For example, the chimeric DR3 protein consist of CRD1 from mouse DR3 and CRD2 to 4 from human DR3 is expressed on CHO cells and the chimeric DR3 can be produced as a soluble Fc fusion protein. If the tested antibody can bind to the human DR3 protein/human DR3 expressed cells, but the antibody can't bind to the CRD1 chimeric DR3 protein/the CRD1 chimeric DR3 expressed cells, it is found that the antibody has the epitope comprising at least one amino acid residue that exists in CRD1 of the extracellular region of DR3 and is the different amino acid residue between human CRD1 and mouse CRD1.

Thereafter, various oligopeptides of the corresponding portions, mutants of the peptides, and the like are synthesized in more detail by using an oligopeptide synthesis technique known to a skilled person in the art, and the reactivity of the target antibody with respect to the peptide is confirmed to identify the epitope. As a simple method of obtaining various oligopeptides, it is possible to use a commercially available kit [for example, SPOTs kit (manufactured by Genosys Biotechnologies), a series of multipin/peptide synthesis kit (manufactured by Chiron) using a multipin synthesis method, or the like].

The antibody which binds to an epitope which is the same as the epitope which the antibody of the present invention binding to the extracellular domain of DR3 recognizes can be obtained by identifying the epitope of the antibody obtained in the binding assay system described above; preparing a partial synthetic peptide, a synthetic peptide which has a three-dimensional structure which mimics that of the epitope, a recombinant protein, or the like of the identified epitope; and performing immunization.

For example, in a case of a membrane protein, a recombinant protein which the entire extracellular domain or a portion of the extracellular domain is fused to an appropriate tag (FLAG tag, His-tag, GST protein, Fc region of an antibody, or the like) is prepared, and the recombinant protein is immunized, whereby an epitope-specific antibody can be prepared more efficiently.

### 2. Preparation of Recombinant Antibody

As production examples of recombinant antibodies, processes for producing a human chimeric antibody and a humanized antibody are shown below.

### (1) Construction of Vector for Expression of Recombinant Antibody

A vector for expression of recombinant antibody is an expression vector for animal cell into which DNAs encoding CH and CL/H and L chains of a human antibody have been inserted, and is constructed by cloning each of DNAs encoding CH and CL/H and L chains of a human antibody into an expression vector for animal cell.

The C region of a human antibody may be CH and CL of any human antibody. Examples include CH belonging to γ1, γ2 or γ4 subclass, CL belonging to κ class, and the like. As the DNAs encoding CH and CL of a human antibody, the cDNA may be generally used and a chromosomal DNA comprising an exon and an intron can be also used. As the expression vector for animal cell, any expression vector can be used, so long as a gene encoding the C region of a human antibody can be inserted thereinto and expressed therein. Examples include pAGE107 [Cytotechnol., 3, 133 (1990)], pAGE103 [J. Biochem., 101, 1307 (1987)], pHSG274 [Gene, 27, 223 (1984)], pKCR [Proc. Natl. Acad. Sci. USA, 78, 1527 (1981)], pSG1bd2-4 [Cytotechnol., 4, 173 (1990)], pSE1UK1Sed1-3 [Cytotechnol., 13, 79 (1993)] and the like. Examples of a promoter and enhancer used for an expression vector for animal cell include an SV40 early promoter [J. Biochem., 101, 1307 (1987)], a Moloney mouse leukemia vitus LTR [Biochem. Biophys. Res. Commun., 149, 960 (1987)], an immunoglobulin H chain promoter [Cell, 41, 479 (1985)] and enhancer [Cell, 33, 717 (1983)] and the like.

The vector for expression of recombinant antibody may be either of a type in which a gene encoding an antibody H chain and a gene encoding an antibody L chain exist on separate vectors (separate type) or of a type in which both genes exist on the same vector (tandem type). Examples of the tandem type of the vector for expression of recombinant antibody include pKANTEX93 (WO 97/10354), pEE18 [Hybridoma, 17, 559 (1998)], Tol2 transposon vector (WO 2010/143698, WO2013/005649) and the like. Further these vectors can be used for separate expression of H and L chains by each gene encoding H or L chain is inserted into a vector, one by one.

(2) Obtaining of cDNA Encoding V Region of Antibody Derived from Non-Human Animal and Analysis of Amino Acid Sequence

Obtaining of cDNAs encoding VH and VL of a non-human animal antibody and analysis of amino acid sequence are carried out as follows.

mRNA is extracted from hybridoma cells producing an antibody derived from a non-human animal to synthesize cDNA. The synthesized cDNA is cloned into a vector such as a phage or a plasmid, to prepare a cDNA library. Each of a recombinant phage or recombinant plasmid containing cDNA encoding VH or VL is isolated from the library using DNA encoding a part of the C region or V region of a mouse antibody as the probe. The full length of the nucleotide sequences of VH and VL of a mouse antibody derived from a non-human animal of interest on the recombinant phage or recombinant plasmid are determined, and the full length of the amino acid sequences of VH and VL are deduced from the nucleotide sequences, respectively.

Examples of the non-human animal for preparing a hybridoma cell which produces a non-human antibody include mouse, rat, hamster, rabbit or the like. Any animals can be used so long as a hybridoma cell can be produced therefrom.

Examples of the method for preparing total RNA from a hybridoma cell include a guanidine thiocyanate-cesium trifluoroacetate method [Methods in Enzymol., 154, 3 (1987)], the use of a kit such as RNA easy kit (manufactured by Qiagen) and the like.

Examples of the method for preparing mRNA from total RNA include an oligo (dT) immobilized cellulose column method [Molecular Cloning, A Laboratory Manual, Second Edition, Cold Spring Harbor Laboratory Press (1989)], a method using a kit such as Oligo-dT30 <Super> mRNA Purification Kit (manufactured by Takara Bio) and the like. Also, examples of a kit for preparing mRNA from a hybridoma cell include Fast Track mRNA Isolation Kit (manufactured by Invitrogen), Quick Prep mRNA Purification Kit (manufactured by Pharmacia) and the like.

Examples of the method for synthesizing cDNA and preparing a cDNA library include known methods [Molecular Cloning, A Laboratory Manual, Cold Spring Harbor Lab. Press (1989); Current Protocols in Molecular Biology, Supplement 1, John Wiley & Sons (1987-1997)]; a method using a kit such as Super Script Plasmid System for cDNA Synthesis and Plasmid Cloning (manufactured by GIBCO BRL), ZAP-cDNA Kit (manufactured by Stratagene), etc.; and the like.

The vector into which the synthesized cDNA using mRNA extracted from a hybridoma cell as the template is inserted for preparing a cDNA library may be any vector, so long as the cDNA can be inserted. Examples include ZAP Express [Strategies, 5, 58 (1992)], pBluescript II SK(+) [Nucleic Acids Research, 17, 9494 (1989)], λzapII (manufactured by Stratagene), λgt10 and λgt11 [DNA Cloning: A Practical Approach, I, 49 (1985)], Lambda BlueMid (manufactured by Clontech), λExCell and pT7T3 18U (manufactured by Pharmacia), pcD2 [Mol. Cell. Biol., 3, 280 (1983)], pUC18 [Gene, 33, 103 (1985)], and the like.

Any Escherichia coli for introducing the cDNA library constructed by a phage or plasmid vector may be used, so long as the cDNA library can be introduced, expressed and maintained. Examples include XL1-Blue MRF′ [Strategies, 5, 81 (1992)], C600 [Genetics, 39, 440 (1954)], Y1088 and Y1090 [Science, 222: 778 (1983)], NM522 [J. Mol. Biol., 166, 1 (1983)], K802 [J. Mol. Biol., 16, 118 (1966)], JM105 [Gene, 38, 275 (1985)], and the like.

A colony hybridization or plaque hybridization method using an isotope- or fluorescence-labeled probe may be used for selecting cDNA clones encoding VH and VL of a non-human antibody or the like from the cDNA library [Molecular Cloning, A Laboratory Manual, Second Edition, Cold Spring Harbor Laboratory Press (1989)].

Also, the cDNAs encoding VH and VL can be prepared through polymerase chain reaction (hereinafter referred to as “PCR”; Molecular Cloning, A Laboratory Manual, Second Edition, Cold Spring Harbor Laboratory Press (1989); Current Protocols in Molecular Biology, Supplement 1, John Wiley & Sons (1987-1997)) by preparing primers and using cDNA prepared from mRNA or a cDNA library as the template.

The nucleotide sequence of the cDNA can be determined by digesting the cDNA selected with appropriate restriction enzymes and the like, cloning the fragments into a plasmid such as pBluescript SK(−) (manufactured by Stratagene), carrying out the reaction by a usually used nucleotide analyzing method. For example, a nucleotide analyze is carried out by using an automatic nucleotide sequence analyzer such as ABI PRISM3700 (manufactured by PE Biosystems) and A.L.F. DNA sequencer (manufactured by Pharmacia) after a reaction such as the dideoxy method [Proc. Natl. Acad. Sci. USA, 74, 5463 (1977)].

Whether the obtained cDNAs encode the full amino acid sequences of VH and VL of the antibody containing a secretory signal sequence can be confirmed by estimating the full length of the amino acid sequences of VH and VL from the determined nucleotide sequence and comparing them with the full length of the amino acid sequences of VH and VL of known antibodies [Sequences of Proteins of Immunological Interest, US Dept. Health and Human Services (1991)]. The length of the secretory signal sequence and N-terminal amino acid sequence can be deduced by comparing the full length of the amino acid sequences of VH and VL of the antibody comprising a secretory signal sequence with full length of the amino acid sequences of VH and VL of known antibodies [Sequences of Proteins of Immunological Interest, US Dept Health and Human Services (1991)], and the subgroup to which they belong can also be known. Furthermore, the amino acid sequence of each of CDRs of VH and VL can be found by comparing the obtained amino acid sequences with amino acid sequences of VH and VL of known antibodies [Sequences of Proteins of Immunological Interest, US Dept Health and Human Services (1991)].

Moreover, the novelty of the full length of the amino acid sequence of VH and VL can be examined by carrying out a homology search with sequences in any database, for example, SWISS-PROT, PIR-Protein or the like using the obtained full length of the amino acid sequences of VH and VL, for example, according to the BLAST method [J. Mol. Biol., 215, 403 (1990)] or the like.

### (3) Construction of Vector for Expression of Human Chimeric Antibody

cDNA encoding each of VH and VL of antibody of non-human animal is cloned in the upstream of genes encoding CH or CL of human antibody of vector for expression of recombinant antibody mentioned in the above (1) to thereby construct a vector for expression of human chimeric antibody.

For example, in order to ligate cDNA comprising a nucleotide sequence of 3′-terminal of VH or VL of antibody of non-human animal and a nucleotide sequence of 5′-terminal of CH or CL of human antibody, each cDNA encoding VH and VL of antibody of non-human animal is prepared so as to encodes appropriate amino acids encoded by a nucleotide sequence of a linkage portion and designed to have an appropriate recognition sequence of a restriction enzyme. The obtained cDNAs encoding VH and VL of antibody are respectively cloned so that each of them is expressed in an appropriate form in the upstream of gene encoding CH or CL of human antibody of the vector for expression of humanized antibody mentioned in the above (1) to construct a vector for expression of human chimeric antibody.

In addition, cDNA encoding VII or VL of a non-human animal antibody is amplified by PCR using a synthetic DNA having a recognition sequence of an appropriate restriction enzyme at both ends and each of them is cloned to the vector for expression of recombinant antibody obtained in the above (1). Furthermore, cDNA encoding H chain or L chain of a recombinant antibody is synthesized or amplified by PCR, and each cDNA is cloned into a vector for expression of recombinant antibody obtained in the above (1).

(4) Construction of cDNA Encoding V Region of Humanized Antibody

cDNAs encoding VH or VL of a humanized antibody can be obtained as follows. Amino acid sequences of framework region (hereinafter referred to as “FR”) in VH or VL of a human antibody to which amino acid sequences of CDRs in VH or VL of an antibody derived from a non-human animal antibody are transplanted are respectively selected. Any amino acid sequences of FR of a human antibody can be used, so long as they are derived from human. Examples include amino acid sequences of FRs of human antibodies registered in database such as Protein Data Bank or the like, and amino acid sequences common to subgroups of FRs of human antibodies [Sequences of Proteins of Immunological Interest, US Dept. Health and Human Services (1991)], and the like. In order to inhibit the decrease in the binding activity of the antibody, amino acid sequences having high homology (at least 60% or more) with the amino acid sequence of FR in VH or VL of the original antibody is selected.

Then, amino acid sequences of CDRs of the original antibody are grafted to the selected amino acid sequence of FR in VH or VL of the human antibody, respectively, to design each amino acid sequence of VH or VL of a humanized antibody. The designed amino acid sequences are converted to DNA sequences by considering the frequency of codon usage found in nucleotide sequences of genes of antibodies [Kabat et al, Sequence of Proteins of Immunological Interest, US Dept. Health and Human Services (1991)], and the DNA sequence encoding the amino acid sequence of VH or VL of a humanized antibody is designed. Based on the designed nucleotide sequences, cDNAs of VH and VL are synthesized, the cDNA encoding VH or VL of a humanized antibody can be easily cloned into the vector for expression of humanized antibody constructed in (1) by introducing the recognition sequence of an appropriate restriction enzyme to the 5′ terminal of the synthetic DNAs existing on the both ends. Otherwise, it can be carried out using a synthetic DNA as one DNA encoding each of the full-length H chain and the full-length L chain based on the designed DNA sequence.

### (5) Modification of Amino Acid Sequence of V Region of Humanized Antibody

It is known that when a humanized antibody is produced by simply grafting only CDRs in VH and VL of an antibody derived from a non-human animal into FRs of VH and VL of a human antibody, its antigen binding activity is lower than that of the original antibody derived from a non-human animal [BIO/TECHNOLOGY, 9, 266 (1991)]. In humanized antibodies, among the amino acid sequences of FRs in VH and VL of a human antibody, an amino acid residue which directly relates to binding to an antigen, an amino acid residue which interacts with an amino acid residue in CDR, and an amino acid residue which maintains the three-dimensional structure of an antibody and indirectly relates to binding to an antigen are identified and modified to an amino acid residue which is found in the parent antibody (original antibody) to thereby increase the decreased antigen binding activity.

In order to identify the amino acid residues relating to the antigen binding activity in FR, the three-dimensional structure of an antibody is constructed and analyzed by X-ray crystallography [J. Mol. Biol., 112, 535 (1977)], computer-modeling [Protein Engineering, 7, 1501 (1994)] or the like. In addition, various attempts must be currently be necessary, for example, several modified antibodies of each antibody are produced and the correlation between each of the modified antibodies and its antibody binding activity is examined. The modification of the amino acid sequence of FR in VH and VL of a human antibody can be accomplished using various synthetic DNA for modification according to PCR as described in (4). With regard to the amplified product obtained by the PCR, the nucleotide sequence is determined according to the method as described in (2) so that whether the objective modification has been carried out is confirmed.

### (6) Construction of Vector for Expression of Humanized Antibody

A vector for expression of humanized antibody can be constructed by cloning each cDNA encoding VH or VL of a constructed recombinant antibody into upstream of each gene encoding CH or CL of the human antibody in the vector for expression of recombinant antibody as described in (1). Further it is also constructed by cloning of cDNA encoding H chain and L chain of the humanized antibody into a appropriate vector. For example, when recognizing sequences of an appropriate restriction enzymes are introduced to the 5′-terminal of synthetic DNAs positioned at both ends among synthetic DNAs used in the construction of VH or VL of the humanized antibody in (4) and (5), cloning can be carried out so that they are expressed in an appropriate form in the upstream of each gene encoding CH or CL of the human antibody in the vector for expression of a humanized antibody as described in (1).

### (7) Transient Expression of Recombinant Antibody

In order to efficiently evaluate the antigen binding activity of various humanized antibodies produced, the recombinant antibodies can be expressed transiently using the vector for expression of humanized antibody as described in (3) and (6) or the modified expression vector thereof. Any cell can be used as a host cell, so long as the host cell can express a recombinant antibody. Generally, COS-7 cell (ATCC CRL1651), CHO-K1 (ATCC CCL-61), CHO-S(Life Technologies, cat. no. 11619) are used in view of its high expression amount [Methods in Nucleic Acids Res., CRC Press, 283 (1991)]. After introduction of the expression vector, the expression amount and antigen binding activity of the recombinant antibody in the culture supernatant can be determined by ELISA, RIA, FCM and the like.

(8) Obtaining Transformant which Stably Expresses Recombinant Antibody and Preparation of Recombinant Antibody

A transformant which stably expresses a recombinant antibody can be obtained by introducing the vector for expression of recombinant antibody described in (3) and (6) into an appropriate host cell. Examples of the method for introducing the expression vector into a host cell include electroporation [Japanese Published Unexamined Patent Application No. 257891/90, Cytotechnology, 3, 133 (1990)] and the like. As the host cell into which a vector for expression of a recombinant antibody is introduced, any cell can be used, so long as it is a host cell which can produce the recombinant antibody. Examples include CHO-K1 (ATCC CCL-61), DUkXB11 (ATCC CCL-9096), Pro-5 (ATCC CCL-1781), CHO-S (Life Technologies, cat. no. 11619), rat myeloma cell YB2/3HL.P2.G11.16Ag.20 (also referred to as YB2/0), mouse myeloma cell NSO, mouse myeloma cell SP2/0-Ag14 (ATCC No. CRL1581), mouse P3X63-Ag8.653 cell (ATCC No. CRL1580), CHO cell in which a dihydrofolate reductase gene (hereinafter referred to as “dhfr”) is defective [Proc. Natl. Acad. Sci. U.S.A., 77, 4216 (1980)], lection resistance-acquired Lec13 [Somatic Cell and Molecular genetics, 12, 55 (1986)], CHO cell in which α1,6-fucosyltransaferse gene is defected (WO 02/31140, WO 2005/35586), rat YB2/3HL.P2.G11.16Ag.20 cell (ATCC No. CRL1662), and the like.

Specific examples thereof can include PER.C6, CHO-K1 (ATCC CCL-61), DUKXB11 (ATCC CCL-9096), Pro-5 (ATCC CCL-1781), CHO-S (Life Technologies, cat. no. 11619), Lec13 cell, rat myeloma cell YB2/3HL.P2.G11.16Ag. 20 (ATCC NO. CRL1662, or also called YB2/0), mouse myeloma cell NS0, mouse myeloma cell SP2/0-Ag14 (ATCC NO. CRL1581), mouse P3X63-Ag8.653 cell (ATCC NO. CRL1580), dihydroforate reductase gene (hereinafter, referred to as dhfr)-deficient CHO cell (CHO/DG44) [Proc. Natl. Acad. Sci. USA, 77, 4216 (1980)], Syrian Hamster cell BHK, HBT563 cell, substrains of the above cell lines and cells prepared by adapting the above cell lines, in serum free medium or under non-adhesion culture conditions, or the like.

In addition, host cells in which activity of a protein such as an enzyme relating to synthesis of an intracellular sugar nucleotide, GDP-fucose, a protein such as an enzyme relating to the modification of a sugar chain in which 1-position of fucose is bound to 6-position of N-acetylglucosamine in the reducing end through α-bond in a complex type N-glycoside-linked sugar chain, or a protein relating to transport of an intracellular sugar nucleotide, GDP-fucose, to the Golgi body are introduced is decreased or deleted, preferably CHO cell in which α1,6-fucosyltransferase gene is defected as described in WO05/35586, WO02/31140 or the like, can also be used.

After introduction of the expression vector, transformants which express a recombinant antibody stably are selected by culturing in a medium for animal cell culture containing an agent such as G418 sulfate (hereinafter referred to as “G418”), cycloheximide (herein after referred to CHX) or the like.

Examples of the medium for animal cell culture include RPMI1640 medium (manufactured by Invitrogen), GIT medium (manufactured by Nihon Pharmaceutical), EX-CELL301 medium, EX-302 medium, EX-325PF (manufactured by JRH), IMDM medium (manufactured by Invitrogen), Hybridoma-SFM medium (manufactured by Invitrogen), media obtained by adding various additives such as fetal calf serum (hereinafter referred to as “FCS”) to these media, and the like. The recombinant antibody can be produced and accumulated in a culture supernatant by culturing the selected transformants in a medium. The expression amount and antigen binding activity of the recombinant antibody in the culture supernatant can be measured by ELISA or the like. Also, in the transformant, the expression amount of the recombinant antibody can be increased by using DHFR amplification system or the like according to the method disclosed in Japanese Published Unexamined Patent Application No. 257891/90.

The recombinant antibody can be purified from the culture supernatant of the transformant by using a protein A column [Monoclonal Antibodies-Principles and practice, Third edition, Academic Press (1996), Antibodies-A Laboratory Manual, Cold Spring Harbor Laboratory (1988)]. For example, the recombinant antibody can be purified by a combination of gel filtration, ion-exchange chromatography, ultrafiltration, protein G column and the like. The molecular weight of the H chain or the L chain of the purified recombinant antibody or the antibody molecule as a whole is determined by polyacrylamide gel electrophoresis (hereinafter referred to as “SDS-PAGE”) [Nature, 227, 680 (1970)], Western blotting [Monoclonal Antibodies-Principles and practice, Third edition, Academic Press (1996), Antibodies-A Laboratory Manual, Cold Spring Harbor Laboratory (1988)], and the like.

### 3. Evaluation of Activity of Purified Monoclonal Antibody or the Antibody Fragment Thereof

The activity of the purified monoclonal antibody of the present invention or the antibody fragment thereof can be evaluated in the following manner. The binding activity with respect to the DR3-expressing cell line can be measured using the binding assay system described in the above section 1 (6) and (7). The CDC activity or the ADCC activity with respect to an antigen positive cell line can be measured by known measurement methods [Cancer Immunol. Immunother., 36, 373 (1993)].

The specific ligand TL1A dependent phosphorylation, activation of DR3 or NF-κB signal and TL1A neutralizing activity by antibodies of the present invention can be measured in the following manner. DR3 expressing cells or human peripheral blood mononuclear cells (PBMC) are sub-cultured in typical medium as RPMI1640 (no FCS) and stabilized for DR3 and NF-κB phosphorylation assay. Cells are incubated with several ng/mL to μg/mL of TL1A in presence or absence of anti-DR3 antibody among several ten minutes, at 37° C., and then cells are lysed or permeabilized. After that, cells are analyzed in phosphorylation level of DR3 and/or NF-κB by western blotting for phosphorylated DR3 and NF-κB. Subsequently extract of the cell is prepared, and the respective proteins are immunoprecipitated using anti-DR3-specific antibody, anti-NF-κB-specific antibody and a house keeping gene (actin or the like)-specific antibody. The precipitated proteins are subjected to electrophoresis using SDS-PAGE, followed by Western blotting by using the DR3-specific antibody and the phosphorylated tyrosine-specific antibody, whereby it is possible to measure the inhibitory activity against phosphorylation of DR3.

Alternatively, the cultured cells to which the antibodies have been added are subjected to protein immobilization and cell membrane permeation treatment by using formaldehyde and saponin, and FCM analysis is carried out using the DR3-specific antibody, the phosphorylated NF-κB specific antibody, or the phosphorylated tyrosine-specific antibody. The phosphorylation of DR3 can also be confirmed in this manner. In addition, regarding trimerization of DR3, culturing and preparation of cell lysate are performed in the same manner as in the test for detecting phosphorylation described above, and then the DR3 proteins are immunoprecipitated using the anti-DR3 antibody so as to detect the precipitated proteins, whereby it is possible to detect trimerization of DR3.

Moreover, TL1A dependent T cell proliferation, a release of inflammatory cytokines as IL-13, GM-CSF, and IFN-γ and the like, are also analyzed in the same manner.

### 4. Method for Treating Disease Using the Anti-DR3 Monoclonal Antibody or Antibody Fragment of the Present Invention

An antibody which specifically recognizes a native conformational structure (three-dimensional structure) of DR3 and binds to the extracellular region, or an antibody fragment thereof of the present invention can be used for treating a disease relating to DR3.

In one embodiment, the antibodies or the antibody composition of the invention can be used to treat, ameliorate or prevent diseases, disorders or symptoms described herein. In preferred embodiments, antibodies of the invention binds to the extracellular domain of DR3, neutralizes the activity of DR3, and doesn't activate DR3 by own binding, are used to treat, prevent or ameliorate inflammatory diseases, autoimmune diseases, cancer diseases and symptoms associated with their diseases.

In preferred embodiments, antibodies, antibody variants or fragments thereof, of the invention that bind the extracellular region of DR3 can be used to treat inflammatory including diseases, Crohn's disease (CD), ulcerative colitis (UC), inflammatory bowel disease (IBD), allergy, acute or chronic reactive airway disease, allergic rhinitis, allergic dermatitis, atopic diseases, atopic asthma, atopic dermatitis, bronchial asthma, eosinophil invasive asthma, chronic obstructive pulmonary diseases (COPD), arthritis, rheumatoid arthritis, systemic lupus erhythematosus (SLE), psoriasis, atherosclerosis, osteoporosis, multiple sclerosis (MS), cancer bone metastasis, blood cancers, solid cancers such as, head and neck cancer, brain cancer, lung cancer, esophageal cancer, gastrointestinal cancer, stomach cancer, intestinal cancer, colorectal cancer, breast cancer, ovary cancer, prostate cancer, uterus cancer, urinary cancer, hepatic cancer, gall bladder cancer, bone cancer and metastasis of cancers.

Autoimmune diseases may be treated by antibodies or antibody composition of the present invention include at least one disease selected from the following: multiple sclerosis, rheumatoid arthritis (RA), autoimmune hemolytic anemia, autoimmune neonatal thrombocytopenia, idiopathic thrombocytopenia purpura (ITP), autoimmunocytopenia, hemolytic anemia, antiphospholipid syndrome, dermatitis, allergic encephalomyelitis, myocarditis, relapsing polychondritis, rheumatic heart disease, neuritis, uveitis ophthalmia, Polyendocrinopathies, purpura (e.g., Henloch-Scoenlein purpura), Reiter's Disease, Stiff-Man Syndrome, autoimmune pulmonary inflammation, Autism, Guillain-Barre Syndrome, insulin dependent diabetes mellitis (IDDM), and autoimmune inflammatory eye disorders, autoimmune thyroiditis, hypothyroidism (i.e., Hashimoto's thyroiditis), systemic lupus erhythematosus (SLE), Goodpasture's syndrome, Pemphigus, Receptor autoimmunities such as, for example, (a) Graves' Disease, (b) Myasthenia Gravis, and (c) insulin resistance, autoimmune thrombocytopenic purpura, rheumatoid arthritis, scleroderma with anti-collagen antibodies, mixed connective tissue disease, polymyositis/dermatomyositis, pernicious anemia, idiopathic Addison's disease, infertility, chronic renal failure (CRF), glomerulonephritis (GN), nephrosis, IgA nephropathy, bullous pemphigoid, Sjogren's syndrome, adrenergic drug resistance (asthma or cystic fibrosis), chronic active hepatitis, primary biliary cirrhosis, other endocrine gland failure, vitiligo, vasculitis, post-MI, cardiotomy syndrome, urticaria, atopic dermatitis, inflammatory myopathies, and other inflammatory, granulamatous, degenerative, and atrophic diseases. Also any complications related to the above identified diseases are also included in the symptoms which the pharmaceuticals of the present invention can treat.

Examples of a route of administration include oral administration and parenteral administration, such as buccal, tracheal, rectal, subcutaneous, intramuscular or intravenous administration. In the case of an antibody or peptide formulation, intravenous administration is preferred. Examples of the dosage form includes sprays, capsules, tablets, powder, granules, syrups, emulsions, suppositories, injections, ointments, tapes and the like.

The pharmaceutical preparation suitable for oral administration includes emulsions, syrups, capsules, tablets, powders, granules and the like. Liquid preparations such as emulsions and syrups can be produced using, as additives, water; sugars such as sucrose, sorbitol and fructose; glycols such as polyethylene glycol and propylene glycol; oils such as sesame oil, olive oil and soybean oil; antiseptics such as p-hydroxybenzoic acid esters; flavors such as strawberry flavor and peppermint; and the like. Capsules, tablets, powders, granules and the like can be produced using, as additives, excipients such as lactose, glucose, sucrose and mannitol; disintegrating agents such as starch and sodium alginate; lubricants such as magnesium stearate and talc; binders such as polyvinyl alcohol, hydroxypropylcellulose and gelatin; surfactants such as fatty acid ester; plasticizers such as glycerin; and the like. The pharmaceutical preparation suitable for parenteral administration includes injections, suppositories, sprays and the like. Injections can be prepared using a carrier such as a salt solution, a glucose solution or a mixture of both thereof. Suppositories can be prepared using a carrier such as cacao butter, hydrogenated fat or carboxylic acid. Sprays can be prepared using the antibody or antibody fragment as such or using it together with a carrier which does not stimulate the buccal or airway mucous membrane of the patient and can facilitate absorption of the compound by dispersing it as fine particles. The carrier includes lactose, glycerol and the like. It is possible to produce pharmaceutical preparations such as aerosols and dry powders. In addition, the components exemplified as additives for oral preparations can also be added to the parenteral preparations.

### 5. Method for Diagnosing Disease Using the Anti-DR3 Monoclonal Antibody or Antibody Fragment of the Present Invention

A disease relating to DR3 can be diagnosed by detecting or determining DR3 or a cell expressing DR3 using the monoclonal antibody or antibody fragment of the present invention. A diagnosis of inflammatory diseases, one of the diseases relating to DR3, can be carried out by, for example, the detection or measurement of DR3 as follows.

The diagnosis of inflammatory diseases can be carried out by detecting DR3 expressing on the cell in a patient's body by an immunological method such as a flow cytometer (FCM). An immunological method is a method in which an antibody amount or an antigen amount is detected or determined using a labeled antigen or antibody. Examples of the immunological method include radioactive substance-labeled immunoantibody method, enzyme immunoassay, fluorescent immunoassay, luminescent immunoassay, Western blotting method, physico-chemical means and the like.

Examples of the radioactive substance-labeled immunoantibody method include a method, in which the antibody or antibody fragment of the present invention is allowed to react with an antigen, a cell expressing an antigen or the like, then anti-immunoglobulin antibody subjected to a radioactive labeling or a binding fragment thereof is allowed to react therewith, followed by determination using a scintillation counter or the like.

Examples of the enzyme immunoassay include a method, in which the antibody or antibody fragment of the present invention is allowed to react with an antigen, a cell expressing an antigen or the like, then an anti-immunoglobulin antibody or an binding fragment thereof subjected to antibody labeling is allowed to react therewith and the colored pigment is measured by a spectrophotometer, and, for example, sandwich ELISA may be used. As a label used in the enzyme immunoassay, any known enzyme label (Enzyme Immunoassay, published by Igaku Shoin, 1987) can be used as described already. Examples include alkaline phosphatase labeling, peroxidase labeling, luciferase labeling, biotin labeling and the like.

Sandwich ELISA is a method in which an antibody is bound to a solid phase, antigen to be detected or measured is trapped and another antibody is allowed to react with the trapped antigen. In the ELISA, two kinds of antibody which recognizes the antigen to be detected or measured or the antibody fragment thereof in which antigen recognizing site is different are prepared and the first antibody or antibody fragments is previously adsorbed on a plate (such as a 96-well plate) and the second antibody or antibody fragment is labeled with a fluorescent substance such as FITC, an enzyme such as peroxidase, or biotin.

The plate to which the above antibody is adsorbed is allowed to react with the cell separated from living body or disrupted cell suspension thereof, tissue or disintegrated solution thereof, cultured cells, serum, pleural effusion, ascites, eye solution or the like, then allowed to react with a labeled monoclonal antibody or an antibody fragment and a detection reaction corresponding to the labeled substance is carried out. The antigen concentration in the sample to be tested can be calculated from a calibration curve prepared by a stepwise dilution of antigen of known concentration. As antibody used for sandwich ELISA, any of polyclonal antibody and monoclonal antibody may be used or antibody fragments such as Fab, Fab′ and F(ab)₂ may be used. As a combination of 2 kinds of antibodies used in sandwich ELISA, a combination of monoclonal antibodies or antibody fragments recognizing different epitopes may be used or a combination of polyclonal antibody with monoclonal antibody or antibody fragments may be used.

A fluorescent immunoassay includes a method described in the literatures [Monoclonal Antibodies—Principles and practice, Third Edition, Academic Press (1996); Manual for Monoclonal Antibody Experiments, Kodansha Scientific (1987)] and the like. As a label for the fluorescent immunoassay, any of known fluorescent labels [Fluorescent Immunoassay, by Akira Kawao, Soft Science, (1983)] may be used as described already. Examples of the label include FITC, RITC and the like.

The luminescent immunoassay can be carried out using the methods described in the literature [Bioluminescence and Chemical Luminescence, Rinsho Kensa, 42, Hirokawa Shoten (1998)] and the like. As a label used for luminescent immunoassay, any of known luminescent labels can be included. Examples include acridinium ester, lophine or the like may be used.

Western blotting is a method in which an antigen or a cell expressing an antigen is fractionated by SDS-polyacrylamide gel electrophoresis [Antibodies-A Laboratory Manual (Cold Spring Harbor Laboratory, 1988)], the gel is blotted onto PVDF membrane or nitrocellulose membrane, the membrane is allowed to react with antigen-recognizing antibody or antibody fragment, further allowed to react with an anti-mouse IgG antibody or antibody fragment which is labeled with a fluorescent substance such as FITC, an enzyme label such as peroxidase, a biotin labeling, or the like, and the label is visualized to confirm the reaction. An example thereof is described below.

Cells or tissues in which a polypeptide having the amino acid sequence represented by SEQ ID NO:2 is expressed are dissolved in a solution and, under reducing conditions, 0.1 to 30 μg as a protein amount per lane is electrophoresed by an SDS-PAGE method. The electrophoresed protein is transferred to a PVDF membrane and allowed to react with PBS containing 1 to 10% of BSA (hereinafter referred to as “BSA-PBS”) at room temperature for 30 minutes for blocking. Here, the monoclonal antibody of the present invention is allowed to react therewith, washed with PBS containing 0.05 to 0.1% Tween 20 (hereinafter referred to as “Tween-PBS”) and allowed to react with goat anti-mouse IgG labeled with peroxidase at room temperature for 2 hours.

It is washed with Tween-PBS and a band to which the monoclonal antibody is bound is detected using ECL Western Blotting Detection Reagents (manufactured by Amersham) or the like to thereby detect a polypeptide having the amino acid sequence represented by SEQ ID NO:2. As an antibody used for the detection in Western blotting, an antibody which can be bound to a polypeptide having no three-dimensional structure of a natural type is used.

The physicochemical method is specifically carried out by reacting DR3 as the antigen with the antibody or antibody fragment of the present invention to form an aggregate, and detecting this aggregate. Other examples of the physicochemical methods include a capillary method, a one-dimensional immunodiffusion method, an immunoturbidimetry, a latex immunoturbidimetry [Handbook of Clinical Test Methods, Kanehara Shuppan, (1988)] and the like. For example, in a latex immunodiffusion method, a carrier such as polystyrene latex having a particle size of about of 0.1 to 1 μm sensitized with antibody or antigen may be used and when an antigen-antibody reaction is carried out using the corresponding antigen or antibody, scattered light in the reaction solution increases while transmitted light decreases. When such a change is detected as absorbance or integral sphere turbidity, it is now possible to measure antigen concentration, etc. in the sample to be tested.

For the detection of the cell expressing DR3, known immunological detection methods can be used, and an immunoprecipitation method, a immuno cell staining method, an immune tissue staining method, a fluorescent antibody staining method and the like are preferably used.

An immunoprecipitation method is a method in which a cell expressing DR3 is allowed to react with the monoclonal antibody or antibody fragment of the present invention and then a carrier having specific binding ability to immunoglobulin such as protein G-Sepharose is added so that an antigen-antibody complex is precipitated. Also, the following method can be carried out.

The above-described antibody or antibody fragment of the present invention is solid-phased on a 96-well plate for ELISA and then blocked with BSA-PBS. When the antibody is in a non-purified state such as a culture supernatant of hybridoma cell, anti-mouse immunoglobulin or rat immunoglobulin or protein A or Protein G or the like is previously adsorbed on a 96-well plate for ELISA and blocked with BSA-PBS and a culture supernatant of hybridoma cell is dispensed thereto for binding. After BSA-PBS is discarded and the residue is sufficiently washed with PBS, reaction is carried out with a dissolved solution of cells or tissues expressing DR3. An immune precipitate is extracted from the well-washed plate with a sample buffer for SDS-PAGE and detected by the above-described Western blotting.

An immune cell staining method or an immune tissue staining method are a method where cells or tissues in which antigen is expressed are treated, if necessary, with a surfactant, methanol or the like to make an antibody easily permeate to the cells or tissues, then the monoclonal antibody of the present invention is allowed to react therewith, then further allowed to react with an anti-immunoglobulin antibody or binding fragment thereof subjected to fluorescent labeling such as FITC, enzyme label such as peroxidase or biotin labeling and the label is visualized and observed under a microscope.

In addition, cells of tissues can be detected by an immunofluorescent staining method where cells are allowed to react with a fluorescence-labeled antibody and analyzed by a flow cytometer [Monoclonal Antibodies—Principles and practice, Third Edition, Academic Press (1996), Manual for Experiments of Monoclonal Antibodies, Kodansha Scientific (1987)] in which cells are allowed to react with a fluorescence-labeled antibody and analyzed by a flow cytometer. Particularly, the monoclonal antibody or antibody fragment of the present invention which binds to an extracellular region of the DR3 can detect a cell expressing the polypeptide maintaining a natural type three-dimensional structure.

In addition, in the case of using FMAT8100HTS system (manufactured by Applied Biosystems) and the like among fluorescent antibody staining methods, the antigen quantity or antibody quantity can be measured without separating the formed antibody-antigen complex and the free antibody or antigen which is not concerned in the formation of the antibody-antigen complex.

The present invention is described below by Examples; however, the present invention is not limited to the following Examples.

### EXAMPLES

### Example 1 Vector Construction

Hereinafter a number of amino acid residue in an antibody or an antibody fragment is defined by EU numbering system Kabat et al (Sequences of Proteins of Immunological Interest, US Dept. Health and Human Services (1991)).

### (1) Expression Vector for Soluble Human Flag-TL1A(EC)

A recombinant soluble human TL1A protein (TNFSF15) (amino acid residues from 71 to 251 of accession NM_005118.2) consisting Flag® Tag (amino acid sequence of DYKDDDDK, hereinafter, in case, solely described as “Flag”) fused to an amino terminal of the extracellular domain (EC) of human TL1A (SEQ ID NO:2) was produced as described following. An “overlapping polymerase chain reaction (PCR)” method was used to produce the cDNA encoding VCAM signal peptide and Flag® Tag sequence fused to hTL1A (EC) (SEQ ID NO:1), while adding flanking restriction sites subsequently used to insert the chimeric cDNA fragment into a mammalian expression vector.

The cDNA coding for human TL1A was isolated from human lymphoma U-937 cell line (ATCC CRL-1593.2). For that, total RNA was isolated from U-937 cells using the RNeasy@ Mini Kit (Qiagen, cat. no. 74104) following manufacturer's instructions. Total cDNA was generated by reverse transcriptase (RT) reaction using the VILO cDNA synthesis kit (Life technologies cat. no. 11754-050), following manufacturer's instructions. Human TL1A cDNA was amplified by PCR using the following primers: hTL1A_F (SEQ ID NO:41) and hTL1A-3′_R (SEQ ID NO:42) with a KOD Hot Start DNA Polymerase kit (Novagen/Toyobo, cat. no. 71086-3) following manufacturer's instructions.

For secretion of the protein from mammalian cells, a signal peptide from the human vascular cell adhesion molecule 1 (VCAM-1) immediately followed by a Flag sequence was introduced to the 5′ end of hTL1A by “overlapping PCR”. A PCR fragment was generated coding for VCAM-Flag with a SalI restriction site added at the 5′ end and 13 nucleotides encoding hTL1A added at the 3′ end with forward and reverse primers VCAM-Flag-SalI-F (SEQ ID NO:43) and VCAM-Flag_hTL1A_R (SEQ ID NO:46), respectively. A second PCR fragment was generated coding for amino acid residues form 71 to 251 of hTL1A (accession NM_005118.2) with 13 nucleotides identical to the end of the Flag-tag added to the 3′ end, and a BamHI site added after at the 3′ end with forward and reverse primers VCAM-Flag®_hTL1A_F (SEQ ID NO:45) and hTL1A-EC_BamHI-R (SEQ ID NO:44), respectively.

The two above PCR products were isolated and added to a third PCR reaction with the forward primer from PCR reaction one (VCAM-Flag-SalI-F (SEQ ID NO:43)) and the reverse primer from reaction two (hTL1A-EC_BamHI-R (SEQ ID NO:44)), resulting in a full length cDNA coding for VCAM-Flag-hTL1A protein (SEQ ID NO:2). This PCR fragment was cloned at SalI and BamHI restriction sites of pKTABEX-TC26 expression vector (WO2013/005649) using DNA Ligase Mighty Mix (Takara, cat. no. 6023) as per the manufacturer's instructions. DNA sequence was confirmed by Sanger sequencing (GENEWIZ, San Diego Calif.). The vector comprising cDNA encoding VCAM-Flag-hTL1A protein was constructed.

### (2) Expression Vector for Soluble Human DR3(EC)-hG1Fc

A soluble chimeric Fc-fusion protein was created that was composed of the amino terminal (N-term) extracellular (EC) domain of human DR3 (TNFRSF25) (NM_003790.2) fused to the Fc domain of human IgG1. A cDNA coding for the extracellular domain of human DR3 was isolated from U-937 cells, as described above for human TL1A, and cDNA encoding the DR3(EC)-hG1Fc protein was produced by overlapping PCR as described in the above Example 1.1 using primers of hDR3_F5′ (SEQ ID NO:47) and hDR3-EC_R (SEQ ID NO:48).

The DNA sequence coding for hDR3-EC (1 to 603 of SEQ ID NO:3, coding for amino acid residues 1 to 201 of SEQ ID NO:4) was amplified by PCR using cDNA as template (described above), the forward primer hDR3-SalI_F (SEQ ID NO:49) and the reverse primer, hDR3_hG1Fc_R (SEQ ID NO:60). The SalI restriction site and the number of nucleotides for the 5′ end of human IgG Fc are introduced to the 5′ end and the 3′ end of the hDR3, respectively. A cDNA for human IgG Fc coding for amino acid residues of 216 to 447 (EU numbering, Kabat et al)(SEQ ID 5 and 6) was amplified by PCR as above, adding 5′ nucleotides homologous to the 3′ end of hDR3(EC), and a 3′ BamHI restriction site using primers hDR3_hG1Fc_F (SEQ ID NO:51) and hG1Fc_BamHI-R (SEQ ID NO:42), respectively. The complete hDR3(EC)-hG1Fc cDNA was generated in a third PCR reaction using combined PCR products from hDR3(EC) and human IgG1 Fc as template and forward primers hDR3-SalI_F (SEQ ID NO:49) and hG1Fc_BamHI-R (SEQ ID NO:52). The resulting PCR fragment was cloned into the SalI and BamHI restrictions sites of pKTABEX-TC26, and the vector comprising cDNA encoding DR3(EC)-hG1Fc protein was produced.

### (3) Expression Vector for Soluble Human DR3(CRD1)-mouseG1Fc

A soluble chimeric Fc-fusion protein was generated comprised of a shorter N-terminal region of human DR3 containing the “cysteine-rich domain (CRD) 1” (amino acid residues of 1 to 71 of SEQ ID NO:8, the full fusion-protein sequence including signal peptide) of human DR3 fused to a mouse IgG1 Fc domain through an intervening short poly-glycine linker peptide (amino acid sequence ASGGGSGGGSGGGS) (SEQ ID NO:8). An “overlapping PCR” method similar to above was used.

The signal peptide and CRD1 of human DR3 (nucleotide sequence of 1 to 213 of SEQ ID NO:7, coding for amino acids 1-71 of SEQ ID NO:8), was PCR amplified from cDNA isolated from U-937 cells, as described above, using forward primer, hDR3-SalI_F (SEQ ID NO:49), and reverse primer, hDR3-pG-NheI_R (SEQ ID NO:53), which adds a 5′ SalI site and a flanking 3′ polyglycine cDNA, respectively. Mouse IgG1 Fc fragment (nucleotide sequence of 256 to 936 of SEQ ID NO:7) was amplified with forward primer, mG1Fc-pG-NheI_F (SEQ ID NO:54) and reverse primer, mG1Fc_BamHI-R (SEQ ID NO:55) which add flanking 5′ polyglycine cDNA and a 3′ BamHI site, respectively. The hDR3(CRD1) and mG1Fc cDNAs were fused in a third PCR, as described above, using primers hDR3-SalI_F (SEQ ID NO:49) and mG1Fc_BamHI-R (SEQ ID NO:55). The PCR fragment was cloned at SalI and BamHI restrictions sites of pKTABEX-TC26 and confirmed by sequencing (GENEWIZ, San Diego Calif.), as described above.

### (4) Vectors for Expressing Surface-Bound Human or Mouse DR3 ΔDD

Vectors were constructed for cell surface expression of human or mouse DR3 protein lacking much of the cytoplasmic “death domain”. The “death domain”, defined variously as amino acid residues from 346 to 417 (Migone et al., Immunity, 2002; 16: 479-492) or from 332 to 412 (Wang et al., Immunogenetics, 2001, 3553:59-63) within the membrane-distal carboxyl-terminal (C-term) region of the cytoplasmic domain of human DR3, is common to other TNFR superfamily members including TNFR1, Fas, DR4, DR5, and DR6, has been shown to be responsible for the observed cytotoxicity of these proteins when DR3 is exogenously over-expressed. This death domain-deleted human DR3, named hDR3ΔDD, (nucleotide sequence of SEQ ID NO:9, coding for the amino acid residues from 1 to 345 of SEQ ID NO:10) was amplified by PCR using forward primers, hDR3-SalI_F (SEQ ID NO:49) and reverse primer hDR3ΔDD_BamHI_R (SEQ ID NO:56), and cDNA of the DR3(EC) previously prepared as a template. The PCR fragment was inserted into pKTABEX-TC26 vector in that the cycloheximide (CHX) resistant gene is replaced with the neomycin resistant gene (NEO), and the vector comprising the cDNA encoding hDR3-ΔDD protein was produced.

Moreover, for the mouse DR3 protein lacking DD, cDNA of mouse DR3 was prepared from a spleen of C57/BL6 mouse using primers, mDR3_Fwd (SEQ ID NO:57) and mDR3_3′_R (SEQ ID NO:58), cDNA encoding mDR3-ΔDD protein was prepared by PCR using forward primer mDR3-SalI_F (SEQ ID NO:59), reverse primer mDR3(1-324)_BamHI_R (SEQ ID NO:60) and mDR3 cDNA as a template. Finally, the vector comprising the cDNA encoding mDR3-ΔDD protein was produced same as the previous method.

### Example 2 Stable Cell Line Generation

### (1) Stable Mouse EL4 Cell Lines Expressing Surface Human DR3ΔDD

A stable mouse cell line expressing surface recombinant human DR3 was created by introducing an expression vector into parental lymphoma cell line, EL4 (ATCC, cat. no. TIB-39). To do this, 1×10⁷ EL4 cells were washed with ice-cold PBS and suspended in 400 μL ice-cold PBS, then to the cell suspension, 10 μg of expression vector for hDR3-ΔDD, and 25 μg Tol2 transposase expression vector (WO2013/005649) were mixed and transferred to a 0.4 cm gap cuvette (Bio-Rad, cat. no 165-2088) and electroporated using a Gene Pulser Apparatus (Bio-Rad, cat. no. 165-2075) [settings: 300 V, 960 μF]. After 24 hours post-transfection, the cells were put under selection with 1 mg/mL geneticin (also described as “G418”, Life Technologies, cat. no. 10131-035) for 19 days. Cells with high cell-surface expression of human DR3 were sorted using FACSAria (BD Biosciences); for that, the cells were stained with biotin anti-human DR3 (TRAMP) antibody (clone JD3 Biolegend, cat. no. 307104) followed by streptavidin-phycoerythrin (Jackson Immunoresearch, cat. no. 016-110-084). The resulting cell line was confirmed at subsequent time-points for stable high surface expression of hDR3 by flow cytometry.

### (2) Stable CHO-K1 Cell Lines Expressing Surface Human or Mouse DR3ΔDD

Suspension CHO-K1 stable cell lines expressing recombinant surface human or mouse DR3 were also generated as described for the EL4-DR3 cell line. Briefly, electroporation of same vectors in the above example 2 (1) was performed using 350V and 500 μF, and the selection at 0.5 to 1 mg/mL G418, was performed for 17 days before a high expressing clone was selected using limiting dilution.

### (3) Soluble Recombinant Protein and Antibody Production by Mammalian Transient Expression

To produce most antibodies and proteins, suspension cultures of FreeStyle™ 293-F cells (Life technologies, cat. no. R790-07) or of FreeStyle™CHO-S cells (Life Technologies, cat. no. R800-07) were transfected with the desired expression vector using FreeStyle™MAX transfection reagent (Life Technologies, cat. no. 16447-100) following manufacturer's instructions and using 1.25 μg vector DNA/μL culture, with a 1:1 (w/v) ratio of DNA to FreeStyle™MAX tratsfection reagent diluted in Opti-MEM medium (Life Technologies, cat. no. 31985-070). Transfectants were subsequently cultured in FreeStyle™ 293 Expression Medium (Life Technologies, cat. no. 12338-018), if using 293-F cells, or FreeStyle™ CHO Expression Medium (Life Technologies, Cat. no. 1265-014), if using CHO-S cells, for six days. Supernatant was clarified by centrifugation followed by 0.22 μm filtration (Millipore, SteriCup® Filter Units). This material was then used directly for assays, or the recombinant protein such as TL1A-flag, DR3-hG1Fc, DR3-mG1Fc, or antibody was purified as described elsewhere in this specification.

(4) Soluble Human DR3(EC)-hG1Fc Production from Stable CHO-K1 Cells

A stable CHO-K1 cell line was generated for larger scale production of soluble human DR3-hG1Fc proteins. 1×10⁷ CHO-K1 cells in 400 μL ice-cold PBS, 10 μg of expression vector pKTABEXTC26-hDR3-hG1Fc and 25 μg Tol2 transposase vector, were transferred to a 0.4 cm gap cuvette and electroporated using a Gene Pulser Apparatus [settings: 350 V, 500 μF]. Cells were immediately transferred to 96-well plates and selection with 3 μg/mL cycloheximide (CHX) (Sigma, cat. no C4859) was started four days later. After 14 to 21 days of selection, wells were screened for soluble IgG Fc by sandwich ELISA. High titer wells were expanded and the cells put through further titer testing until final high-expressing line was selected.

Production of human DR3 (EC)-hG1Fc from the CHO-K1 stable cell line was performed using a fed-batch production method using CHO CD Efficient Feed A and Feed B (Life Technologies cat. no. A10234-01 and cat. no. A10240-01 respectively). The cells were scaled-up to the desired volume, and fed with a mixture of Feed A and Feed B. Culture supernatant was harvested when cell's viability fell below 80%, and was clarified by centrifugation followed by 0.22 μm filtration.

### Example 3 Establishment of Anti-DR3 Monoclonal Antibody

### (1) Mouse Immunization

Kirin-Medarex (KM) mice (containing human Ig gene loci) (WO 02/43478, WO 02/092812, Ishida, et al., IBC's 11.sup.th Antibody Engineering Meeting. Abstract (2000); Kataoka, S. IBC's 13.sup.th Antibody Engineering Meeting. Abstract (2002)) were immunized with 50 μg human DR3-Fc fusion protein or DR3-expressing EL4 cells in Sigma Adjuvant System (SAS) (cat. no. S6322) on day 0, 14, and 21. Serum anti-hDR3 titers were typically evaluated via flow cytometric binding to human DR3-expressing CHO cells. Mice with appropriate titers (typically displaying endpoint dilutions of 1:10,000) were immunized one final time with DR3-Fc or DR3-EL4 cells. Mice were sacrificed after 3 days following final boost.

### (2) Hybridoma Generation

Spleens from immunized mice were excised, and single-cell splenocyte suspensions were prepared. Splenocytes were mixed 1:5 with Sp2/0-Ag14 (ATCC CRL-1581) fusion partner cells, and washed 3 times with 20 mL warmed DMEM (Invitrogen). Medium was aspirated from the final wash, and 1 mL warmed PEG1500 (Boehringer Mannheim) was added dropwise to cells over 1 minute with gentle mixing. Resulting PEG1500 cell suspensions were gently mixed for an additional 2 minutes, followed by a series of dropwise warmed DMEM additions (1 mL over 1 min., then 3 mL over 3 min., then 10 mL over 1 min.), all with gentle swirling.

Fusion mixtures were incubated for 5 minutes at 37° C. Cells were pelleted via centrifugation and resuspended at 10⁶ cells/mL in DMEM supplemented with 10% fetal bovine serum (FBS), 50 pg/mL recombinant IL-6 (R&D Systems 206-IL-050). Fusion mixtures were distributed at 100 UL/well in 96-well tissue culture plates and cultured overnight. The following day, 100 ILL DMEM 2×HAT-supplement (Sigma Aldrich) containing medium (hereinafter described as DMEM/HAT) was added, and plates were cultured an additional 3 days. 75% of culture medium was replaced with 1×DMEM/HAT, and hybridoma cells were cultured an additional 2 to 4 days.

### (3) Antibody Selection

Supernatants of multiwell plates were screened by flow cytometric binding to human DR3-expressing CHO cells (hereinafter described as CHO/DR3, in case). Cells were incubated at 10,000/well in a 96-well round-bottom assay plate for 15 minutes at 4° C. with a neat supernatant of hybridoma. Cells were pelleted via centrifugation, and washed with 200 μL PBS/0.5% fetal calf serum. After subsequent centrifugation, cells were resuspended in 200 μL PBS/0.5% fetal calf serum containing 10 μg/mL anti-human IgG-phycoerytherin (Jackson Immuno Research, 109-115-098), and incubated for 15 minutes at 4° C. Cells were washed as described above, and resuspended in 200 μL PBS/0.5% fetal calf serum; >5000 events acquired on an LSR Fortessa flow cytometer (Beckton Dickinson). Results were analyzed using FlowJo (Treestar Inc.). As a result, hybridoma clones including 142A2 and 142S38B which produce anti-DR3 monoclonal antibodies, were established.

DR3 binding antibody-producing hybridoma lines were expanded and an antibody of supematant was purified according to conventional methodology. Antibody protein purification: Human monoclonal antibodies were purified from culture media using recombinant MabSelect SuRe Protein A affinity resin (GE Healthcare Life Sciences, Pittsburgh, Pa.). The conditioned medium was filtered with a 0.22 μm vacuum filter unit (Millipore, Bedford, Mass.) and loaded onto a Protein A column (GE Healthcare Life Sciences, Pittsburgh, Pa.) of appropriate capacity to match the amount of human antibody in the medium. The column was washed thoroughly with 5 column volumes of PBS, the antibody was eluted with 0.1 M Gly-HCl, pH 3.6, and neutralized with 1 M Tris-HCl, pH 8.0.

The fractions were analyzed by SDS-PAGE and the positive fractions were pooled and dialyzed against PBS pH 7.4 (Gibco, Life Technologies, Grand Island, N.Y.). Following dialysis, antibody samples were concentrated with a centrifugal concentrator (Vivaspin, 50,000 MWCO: Sartorius, Gettingen, Germany). Finally, the antibody was filter sterilized using syringe filters with 0.22 m pore diameter and the antibody concentration was determined by the Lowry method. Pyrogen content was determined using FDA-licensed Endosafe-PTS Limulus Amebocyte Lysate (LAL) assay (Charles River Laboratories). The limits of detection of this assay are 0.01 EU/mL. If the test was negative, the samples were considered endotoxin free.

Purified antibodies were evaluated for DR3 agonism/antagonism as described below. Through several fusions, we established anti-human DR3 monoclonal antibodies including 142A2 and 142S38B and these two antibodies were conducted to gene cloning and further characterization.

### Example 4 Gene Cloning of Anti-DR3 Monoclonal Antibodies

(1) Gene Cloning and Sequencing of VH and VL Genes from Hybridomas 142A2 and 142S38B

The cDNA coding for a signal peptide and rearranged immunoglobulin heavy chain variable (VH) domains (VH herein referring to the combined variable gene (V)+diversity gene (D)+joining gene (J) regions from human genome), and a signal peptide and immunoglobulin light chain variable (VL) domains (VL herein referring to the combined variable gene (V)+joining gene (J) regions from human genome) from selected hybridoma cell lines 142A2 and 142S38B were extracted using 5′-SMART-RACE-PCR (Switching Mechanism at 5′ End of RNA Template-Rapid Amplification of cDNA Ends-Polymerase Chain Reaction) and Sanger-sequenced.

To do this, total RNA was purified from each hybridoma cells using an RNeasy® kit (QIAGEN Inc., cat. no. 74104) following the manufacturer's instructions. Using the isolated RNA as a template, a SMART RACE cDNA Amplification Kit (Clontech, cat. no. 634914), for 142A2, or SMARTer RACE cDNA Amplification Kit (Clontech, cat. no. 634923) combined with the reverse transcriptase SuperScript™II (Invitrogen, cat. no. 18064-014), for 142S38B, were used to amplify the VH and VL domains. Following the manufacturer's instructions, first strand cDNA was generated with the kit-derived primers and reverse transcriptase from 2 μg of hybridoma-derived total RNA.

This cDNA was used as a template for PCR amplification of the VH or VL region and a short part of the constant region of heavy or light chain, using gene-specific reverse primers annealing within the constant domain of all human gamma isotypes (primer IgG1, SEQ ID NO:61) or human kappa (primer hk-5, SEQ ID NO:62), in combination with the kit-provided universal forward primer. KOD Hot Start DNA polymerase (Novagen/Toyobo, cat. no. 71086-3) was used within following thermal-cycling program: 94° C. 4 min; 5 cycles of 94° C. 30 sec., 68° C. 2 min; 5 cycles of 94° C. 30 sec., 66° C. 30 sec., 68° C. 1 min.; 25 cycles of 94° C. 30 sec., 64° C. 30 sec., 68° C. 1 min.; once cycles of 68° C. 5 min. Insufficient 142A2 VH cDNA was isolated from this reaction, so a second round of partial-nested PCR was performed using DNA from the first PCR reaction as template. The same kit-proved forward primer was paired with a nested reverse primer annealing within the heavy constant domain (primer IgG2, SEQ ID NO:71) and the PCR was repeated as before. All other antibody fragment did not require a second round PCR. Amplified VH and VL cDNA fragments were cloned into PCR Blunt II-TOPO plasmid using the Zero Blunt TOPO PCR Cloning Kit (Invitrogen, cat. no. K2820) following the manufacturer's instructions.

Multiple plasmids containing VH or VL cDNA inserts were sequenced by Sanger-sequencing (performed by GENEWIZ Inc.), aligned using the program Sequencher (manufactured by Gene Codes Corp.). Consensus alignments for VH and VL cDNA and analyze using IMGT/V-quest program (Brochet, Lefranc et al. 2008), BLAST and IgBLAST. Complementarity determining regions (CDRs) of an antibody were identified using the Kabat's definition (Kabat et al, Sequences of Proteins of Immunological Interest, US Dept. Health and Human Services (1991)).

The identified sequences were as follows: 142A2 VH (the nucleotide sequence of SEQ ID NO:13, the amino acid sequence of VH region including the signal peptide of SEQ ID NO:14, the amino acid sequence of VH region of SEQ ID NO:15 and HCDRs 1 to 3 of SEQ ID NOs: 16 to 18), 142A2 VL (the nucleotide sequence of SEQ ID NO:19, the amino acid sequence of VL region including the signal peptide of SEQ ID NO:20, the amino acid sequence of VL of SEQ ID NO:21, and LCDRs 1 to 3 of SEQ ID NOs:22 to 24); 142S38B VH (the nucleotide sequence of SEQ ID NO:25, the amino acid sequence of VH region including the signal peptide of SEQ ID NO:26, the amino acid sequence of VH region of SEQ ID NO:27 and HCDRs 1 to 3 of SEQ ID NOs:28 to 30), 142S38B VL (the nucleotide sequence of SEQ ID NO:31, the amino acid sequence of VL region including the signal peptide of SEQ ID NO:32, the amino acid sequence of VL of SEQ ID NO:33, and LCDRs 1 to 3 of SEQ ID NOs:34 to 36).

(2) Cloning of VH and VL into IgG1, IgG2 and IgG4 Expression Vectors.

Vectors for expression of full length human IgG1, IgG2 or IgG4 variant antibody versions of both 142A2 and 142S38B were constructed by ligating the identified cDNA coding for the VH or VL domains, including endogenous signal peptide, into expression vectors containing each human γ constant region (IgG1, IgG2 or IgG4 variant) or human kappa constant domain (SEQ ID NO:37), respectively. At this example, IgG4 variant refers as Nullbody® that comprises amino acid substitutions of S228P, L235E and R409K in the human IgG4 constant region (the number of amino acid position defined by EU index) (US2008-0063635). In the case, Nullbody® type antibody is referred to IgG4-null. The IgG4 antibody variant is the antibody that shows an increased stability in lower pH or acidic condition with keeping an original binding property of an IgG4 antibody, and as a result, the antibody is not almost aggregated through antibody purification processes. The ligation of cDNA was done such that the resulting translated amino acids at the variable-to-constant junction were unchanged from the hybridoma-derived sequence.

To do this, the signal peptide and the VH domains were amplified using a forward primer adding a 5′ flanking SalI restriction site [primer 142A2-VH_SalI_F (SEQ ID NO:67) for 142A2, and primer 142S38B_VHF_SalI (SEQ ID NO:69) for 142S38B)], and reverse primer adding a 3′ flanking NheI restriction site [(primer 142A2-VH_NheI_R (SEQ ID NO:68) for 142A2, and primer F429_VH_R1 (SEQ ID NO:70) for 142S38B)]. The PCR product was cloned into SalI and NheI restriction sites within expression vector pKTABEX-TC26 which contained cDNA for the immunoglobulin constant domain of desired isotype [amino acid sequence for human IgG1 (SEQ ID NO:38), IgG2 (SEQ ID NO:39), or IgG4-null (SEQ ID NO:40)]. Similarly, VL domains for each antibody were amplified and inserted into BglII and BsiWI restriction sites of the same, or another, vector containing cDNA for the constant domain of human kappa. PCR primers used for amplifying the signal peptide and VL and adding 5′ and 3′ flanking BglII and BsiWI restriction sites were as follows: for 142A2, forward primer 142A2_BglII_F (SEQ ID NO:65) and reverse primer 142A2_BsiWI_R (SEQ ID NO:66); for 142S38B, forward primer 142S38B_VLF_Bglll (SEQ ID NO:63) and reverse primer: 142P20_VL1_BsiWI_R (SEQ ID NO:64). Transient production of antibodies was performed using CHO-S cells as described in the above example 2 (3).

### Example 5 Biacore-Based Evaluation of Binding Activity of Anti-DR3 Antibodies to Recombinant Human DR3-Human IgG1-Fc (huDR3-huFc)

In order to kinetically analyze the binding activity of anti-DR3 human antibodies 142A2 and 142S38B to the recombinant human DR3 protein, the binding activity of the antibody was measured by surface plasmon resonance method (SPR). Fab antibody fragments of the above mentioned antibodies were obtained following the Pierce Fab Preparation Kit (Thermo Scientific Pierce No. 44985). All of the following manipulations were carried out using a Biacore® 3000 (manufactured by GE Healthcare Bio-Sciences). Recombinant huDR3-huFc protein was immobilized on a CM5 sensor chip (manufactured by GE Healthcare Bio-Sciences) by an amine coupling chemistry.

In particular, the kinetic assay was carried out by immobilizing on the chip approximately 2000 resonance unit (RU) of recombinant protein, in order to achieve a medium protein immobilization level. Thereafter, the anti-DR3 antibody Fab fragments, diluted from 12 nM concentration in five steps, were allowed to run at a flow rate of 30 μL/min onto the chip for 120 seconds. The dissociation time was 300 seconds and the binding curves were measured at 25° C.

Regeneration was performed with glycine-HCl, pH 1.5, 60 seconds. The raw data were double referenced by subtraction of the signals from a reference flow cell without captured ligand and a buffer blank. The sensorgram corresponding to each concentration was obtained. The analysis was carried out using a 1:1 Langmuir fit model, using the analysis software attached to the apparatus, Biacore® 3000 Evaluation software (manufactured by Biacore), thereby calculating an association rate constant ka [1/Ms] and a dissociation rate constant kd [1/s] for the recombinant huDR3-huFc protein.

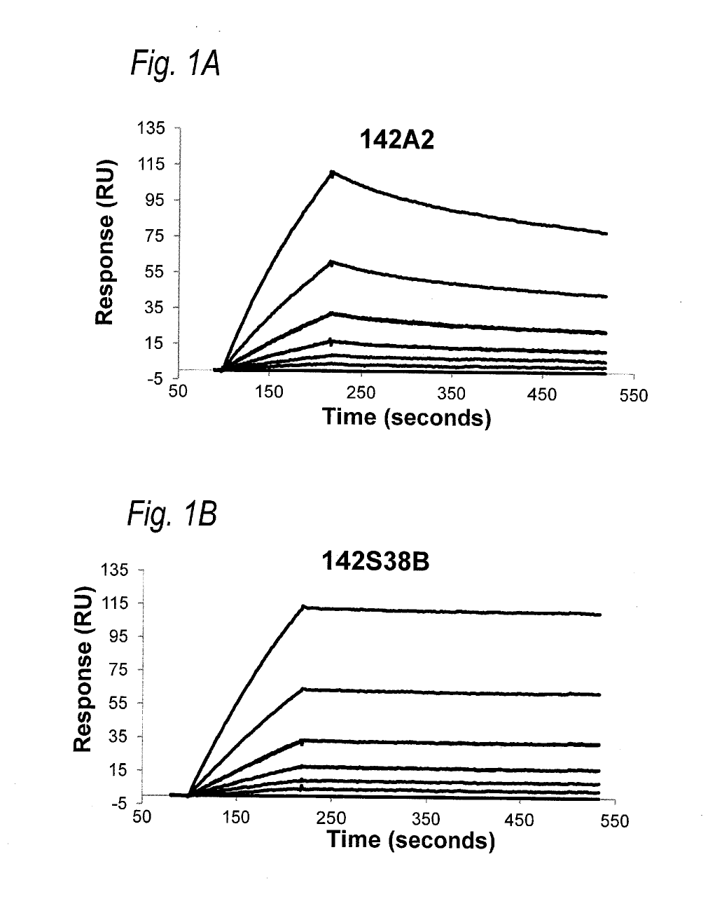

As a result, an equilibrium dissociation constant K_{D} (kd/ka) of individual antibodies thus obtained is given in Table 1. A typical sensorgram of each antibody is depicted in FIG. 1A and FIG. 1B. According to the analysis, anti-DR3 monoclonal antibodies 142A2 and 142S38B had significantly higher affinity for the human DR3 protein at the magnitude of K_{D} value more than 10⁹ orders. Furthermore, the 142A2 antibody had a higher association rate constant (ka) than the 142S38B antibody, on the other hand the 142S38B antibody had a higher dissociation rate constant (kd) than the 142A2 antibody.

TABLE 1
Affinity analysis of anti-DR3 monoclonal antibodies

| Antibody type | 142A2 Fab | | 142S38B Fab | |
|---|---|---|---|---|
| Concentrations (nM) | 12 to 0.375 | | | |
| ka (1/Ms) | 2.88e5 | 7.39e5 | 2.39e5 | 3.37e5 |
| kd (1/s) | 1.09e−3 | 1.09e−3 | 5.37e−5 | 5.46e−5 |
| K_{D} (M) | 3.79e−9 | 1.5e−9 | 2.25e−10 | 1.62e−10 |
| Average K_{D} | 2.65e−09 | | 1.94e−10 | |
| (M) n = 2 | | | | |
| Rmax (RU) | 334 | 142 | 427 | 297 |
| Chi² | 0.441 | 0.956 | 0.0471 | 0.118 |

### Example 6 DR3 Agonism and Antagonism of Anti-DR3 Monoclonal Antibodies

In order to evaluate agonism and antagonism of anti-DR3 monoclonal antibodies established in the present invention, a level of phosphorylation of p65 (RelA) NF-κB subunit in human peripheral blood mononuclear cells (PBMC) is analyzed by FCM method. PBMCs were purified from healthy volunteers by differential density centrifugation using Ficoll. To stabilize basal NF-κB activation levels in PBMCs, PBMCs were rested at 37° C. for 30 minutes in serum-free RPMI1640 medium (Invitrogen) (hereinafter described as RPMI).

For the comparison of isotype effects on antagonist and agonist activity of anti-DR3 monoclonal antibody. The effects of indicated 142A2 IgG1, IgG2 and IgG4 variant versions on PBMC NF-κB activation were determined as described in the below. In agonist assay, cells were treated with 6.67 nM anti-DR3 monoclonal antibody 142A2-IgG1, 142-A2-IgG2 or 142A2-IgG4v alone, or 40 nM TL1A-flag alone as a positive control, for 30 minutes at 37° C. in RPMI. In antagonist assay, cells were pretreated with presence or absence of 6.67 nM anti-DR3 monoclonal antibody 142A2-IgG1, 142-A2-IgG2 or 142A2-IgG4v, for 30 minutes at 37° C. in RPMI, and then 40 nM TL1A-flag was added and cells were incubated for 10 minutes at 37° C. in RPMI. After each incubation, cells were stained with anti-phospho p65 (Becton, Dickinson, cat. no. 558423), and analyzed using a Becton Dickinson LSR Fortessa flow cytometer.

For antagonist assays on dose dependency of antibodies, cells were then treated with presence or absence of anti-DR3 monoclonal antibodies at each concentration for 30 minutes at 37° C. in RPMI. After that, 1 μg/mL (40 nM) exogenous TL1A-flag was added into the medium for 10 minutes in presence of antibodies, followed by fixation with Becton Dickinson (BD) Cytofix and permeabilization with BD PhosFlow Permeablization Buffer III (Becton Dickinson, cat. 554714).

For agonist assays on dose dependency of antibodies, cells were treated with antibodies alone, the negative control of RPMI alone, or the positive control of 40 nM recombinant TL1A for 10 minutes, followed by fixation and permeabilization as described above. Cells were stained with anti-phospho p65 (Becton, Dickinson, 558423), and analyzed using a Becton Dickinson LSR Fortessa flow cytometer.

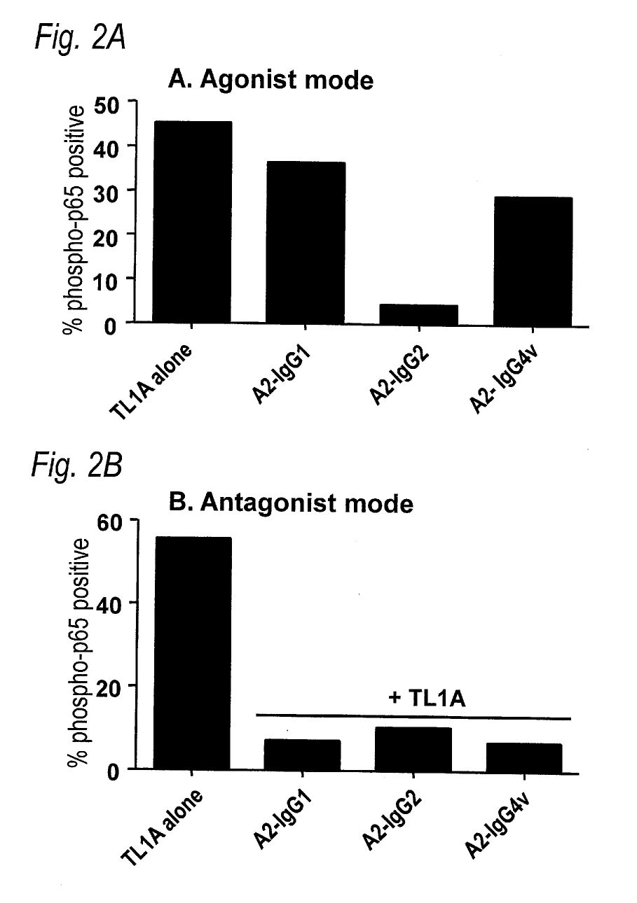

As shown in FIG. 2A, TL1A alone as the positive control, increased the phosphorylated p65 positive cells in PBMCs to approximately 45%, on the other hand, the anti-DR3 monoclonal antibody 142A2-IgG1 and anti-DR3 monoclonal antibody 142A2-IgG4v also increased phosphorylated p65-positive cells up to about 80% or 70% of the control. However anti-DR3 monoclonal antibody 142A2-IgG2 merely increased the phosphorylated p65-positive cells less than one tenth of the control. Therefore, for the bivalent IgG antibody format, it was found that a rearrangement of IgG2 class can decrease or delete the agonism potency by anti-DR3 monoclonal antibody binding. Thus anti-DR3 monoclonal antibody of IgG2 class can be useful for pure antagonism of DR3 antigen in a treatment for diseases caused by aberrant DR3 activation.

Moreover, as shown in FIG. 2B, comparing to TL1A increased phosphorylated p65 positive cells in PBMCs at approximately 55%, all anti-DR3 monoclonal antibodies of IgG1, IgG2 and IgG4 almost completely inhibited the phosphorylation of p65 induced on TL1A binding. Therefore it was shown that the anti-DR3 monoclonal antibody of IgG2 class has an unexpected property which shows the antagonism of DR3 with the lowered or no agonism. These experimental results clearly show that anti-DR3 monoclonal antibody of IgG2 class can be selected as one candidate of pure antagonist for diseases related to aberrant DR3 activation.

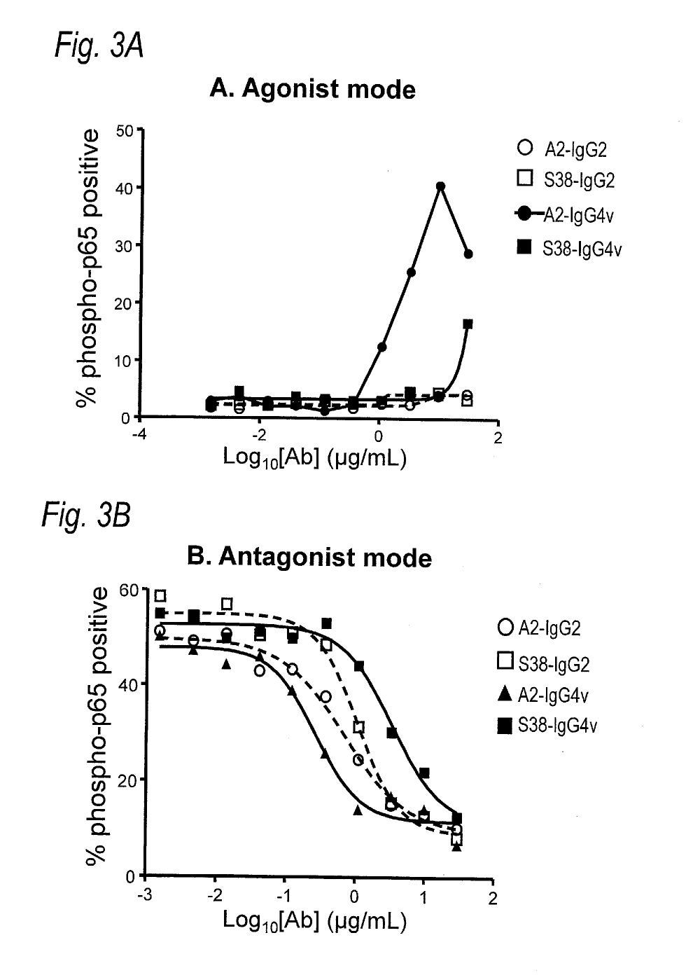

On the other hand, as shown in FIG. 3A, anti-DR3 IgG4 antibody variants 142A2 and 142S38B increased phosphorylation of p65 in PBMCs, on the antibody dose dependent manner, thus these antibodies showed agonistic activity for DR3 and the agonistic activity of the A2-IgG4v was higher activity than S38-IgG4v. On the other A2-IgG2 and S38-IgG2 that have the constant region of IgG2 didn't increase the phosphorylation of p65 in PBMCs, even if in the highest antibody concentration. Thus these IgG2 antibodies lacked an agonistic activity.

Moreover, as shown in FIG. 3B, all anti-DR3 monoclonal antibodies 142A2 and 142S38B that have IgG2 or IgG4 variant constant region decreased TL1A-induced p65 phosphorylation, and thereby demonstrated antagonistic activity for DR3. The antagonistic activity of 142A2 antibodies showed higher antagonistic activity compared to that of 142S38B antibodies. The rearrangement to IgG2 subclass from IgG4 subclass slightly decreased the antagonist potency of 142A2 antibody, on the other hand it slightly increased the antagonist potency of 142S38B antibody.

In contrast to previously known bivalent anti-DR3 antagonistic antibodies, the IgG2 rearranged anti-DR3 antibodies retains DR3 antagonism, but decreased or lacked significant agonist activity. Thus, bivalent IgG2 would be useful for inhibiting DR3 function without significantly stimulating DR3.

### Example 7 Antagonism of TL1A-Mediated IL-13 Production Inhibition

In order to analyze a secretion of inflammatory cytokines from T cells in the presence or absence of anti-DR3 monoclonal antibodies, the secretion of IL-13 as one of inflammatory cytokines was assayed. T cells were purified from human PBMCs (isolated via Ficoll as described above) by magnetic activated cell sorting using Miltenyi Pan-T Cell Isolation Kit II (Miltenyi Biotech 130-095-130). Isolated T cells were seeded onto 96-well anti-CD3-coated plates (2×10⁵ cells/well; eBioscience cat. no. 16-0037-85) with 1 μg/mL anti-CD28 antibody (Becton Dickinson, cat. no. 556620) and 1 μg/mL recombinant TL1A-flag prepared in the above example 2 (4). Various concentrations of anti-DR3 antibodies were added, and cells were cultured for 72 hrs at 37° C. Supernatant IL-13 levels were assessed by ELISA (R&D Systems, cat No. D1300B).

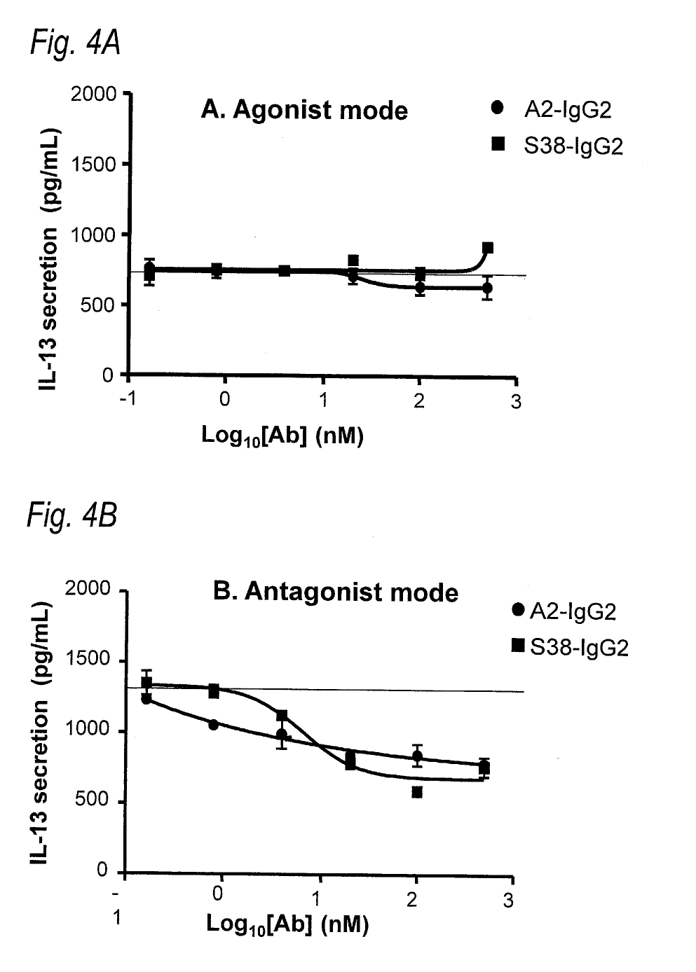

As shown in FIG. 4A, anti-DR3 monoclonal antibodies 142A2-IgG2 and 142S38B-IgG2 didn't increase the secretion of IL-13 from PBMCs, amount secreted was the same as IL-13 secretion in the medium alone. Therefore, in accordance with the results in analysis of p65 phosphorylation, it was shown that the anti-DR3 monoclonal antibodies of IgG2 class did not have any agonistic potency for the inflammatory cytokine releases. Furthermore, as shown in the FIG. 4B, anti-DR3 monoclonal antibodies 142A2-IgG2 and 142S38B-IgG2 inhibited TL1A-induced secretion of IL-13 from PBMCs in does dependent manner.

Therefore, in accordance with the above analysis of anti-DR3 monoclonal antibodies of IgG2 class, the rearrangement of IgG2 class completely canceled agonism potency of anti-DR3 monoclonal antibodies. Thus, the anti-DR3 monoclonal antibody of IgG2 class can be one pure antagonist for diseases related to inflammatory cytokine releases by aberrant DR3 activation.

### Example 8 Preparation of Anti-DR3 Monoclonal Antibody Fragments

(1) Construction of the Anti-DR3 monovalent Antibodies

In order to produce the antibody fragment comprising the Fc region of the antibody, the monovalent antibody (hereinafter described as mvAb, in case) composed of a H chain and a Fc is fused to L chain (FL fusion polypeptide) was designed as one antibody fragment. The anti-DR3 monoclonal antibodies 142A2 and 142S38B are used as a parent antibody clones for constructing an anti-DR3 monovalent antibodies, the amino acid sequence of the VH of the antibody was linked to an amino acid sequence of a constant region of human IgG4 antibody in which C131S R133K, S228P, L235E and R409K substitutions are included, and thereby the H chain for mvAb was constructed.

Then the amino acid sequence of the VL of the antibody was linked to the amino acid sequence of a constant region of human kappa light chain following the Fc region of human IgG4 antibody comprising the hinge, CH2 and CH3 domains in which C214S, S228P, L235E, R409K, H435R and Y436F substitutions are included, and thereby FL fusion polypeptide was constructed.

Each amino acid sequence of VH and VL derived from 142A2 antibody or 142S38B antibody (SEQ ID NOs: 15 and 21, or 27 and 33) was conjugated to the amino acid sequence of IgG4 heavy chain constant region comprising C131S, R133K, S228P, L235E and R409K substitutions (142A2/mvG4_HC; SEQ ID NO:72 and 142S38B/mvG4_HC; SEQ ID NO:73) or the amino acid sequence of a constant region of human kappa light chain following the Fc region of human IgG4 heavy chain consist of the hinge, CH2 and CH3 domains comprising C214S, S228P, L235E, R409K, H435R and Y436F substitutions (142A2/mvG4_LC; SEQ ID NO:74 and 142S38B/mvG4_LC; SEQ ID NO:75), respectively. Each pair of H chain and FL-fusion polypeptide composes an anti-DR3 monovalent antibody mv142A2 and an anti-DR3 monovalent antibody mv142S38BA.

A DNA sequence encoding the amino acid sequence of 142A2/mvG4 HC (SEQ ID NO:72) or 142S38B/mvG4_HC (SEQ ID NO:73) in which each sequence recognized by a restriction enzyme NotI and BamHI are introduced at a 5′-terminal and a 3′-terminal, was respectively inserted into the NotI and BamHI sites on an expression vector pKANTEX93 (WO 97/10354).

Further a DNA sequence encoding the amino acid sequence of 142A2/mvG4_LC (SEQ ID NO:74) or 142S38B/mvG4_LC(SEQ ID NO:75) in which each sequence recognized by a restriction enzyme EcoRI and KpnI are introduced at a 5′-terminal and 3′-terminal, was respectively inserted at the expression vector pKANTEX93 comprising FL chain polypeptide. As the result, expression vectors of anti-DR3 monovalent antibody, pKANTEX93/142A2/mvG4 and pKANTEX93/142S38B/mvG4 were constructed.

### (2) Production and Purification of the Anti-DR3 Monovalent Antibodies

In order to produce the antibody fragment comprising the Fc region of the antibody, a Chinese hamster ovary CHO/DG44 cell (Somatic Cell Mol. Genet., 12, 555, 1986) was used. Cell culture was performed at 37° C. in a 5% CO₂ incubator. In order to express a variety of monovalent antibodies, introduction of the expression vectors was performed in the following manner.

8 μg of various monovalent antibody expression vectors were added to 2.0×10⁷ of CHO/DG44 cells, and gene was introduced by electroporation method [Cytotechnology, 3,133(1990)]. A cuvette of 2 mm gap was kept on ice for 5 min, then the electric pulse under condition of 350 V, 250 μF was conducted. After the pulse, cells were recovered with 15 mL of IMDM medium (manufactured by GIBCO) [hereinafter, abbreviated to IMDM-(10)] containing 10% dialyzed fetal bovine serum (hereinafter, abbreviated to dFBS) and each cell was cultured in an IMDM-(10) for 2 days, and then the medium was replaced with IMDM-(10) [hereinafter, abbreviated to IMDM-(10G)] containing 0.5 mg/mL G418 sulfate (NACALAI TESQUE, INC.) to continue the culture, thereby obtaining a G418 resistant cell line.

G418 resistant cell line obtained under previous cultivation was suspended in IMDM-(10G) and cultured for a while of being confluent in the 175 cm² flask, and then cells were moved to the HYPERflask™ (Corning, cat. no. 10020) and cultured in Excell302 (manufactured by SAFC Biosciences) supplemented with 0.5 mg/mL G418 and 10 μg/mL gentamicin for 7 to 11 days and then culture supernatant was recovered. For the anti-DR3 monovalent antibody 142S38B/mvG4, the transient protein expression system similarly described in the section (3) of Example 2 was carried out. As the result of cultivation of transfected cells, culture supernatants of 142A2mvG4 and 142S38BmvG4 were obtained.

For purification of mvAbs, the culture supernatants of the various monovalent antibodies in the above were passed through a 0.5 mL volume column packed with Mab Select SuRe (manufactured by MILLIPORE) carrier at a flow rate of 0.5 to 1.0 mL/min. The column was washed with phosphate buffer saline (PBS) twice, and for the 142A2/mvG4, 0.1 M citrate buffer (pH4.0), for the 142S38B/mvG4, 0.1M citrate buffer (pH3.9) was respectively used for each elution, and the each eluted fraction was immediately neutralized with 2 M Tris-hydrochloric acid buffer (pH 8.0).

The elution fraction showing a high protein concentration was dialyzed twice against a buffer (hereinafter, abbreviated to citrate buffer) containing 10 mM citric acid, of which pH was adjusted to 6.0 with sodium hydroxide, and 150 mM sodium chloride. The sample was recovered, and a low-concentration sample was concentrated by an ultrafiltration filter (manufactured by MILLIPORE), and sterilized using a 0.22 μm filter (manufactured by MILLIPORE).

The sample was further purified by gel filtration chromatography, and used in the in vitro activity test. A Superdex 200 10/300 GL column (GE Healthcare) was connected to a High speed liquid chromatography system AKTA explore 10S (GE Healthcare), and the aqueous solution in the column was changed with the citrate buffer, and the buffer was used as a running buffer. 550 μL of prepared volume of antibody solution was applied to the column in a manual, and the buffer of 1.5 column volume was passed through the column at a flow rate of 0.5 mL/min (tolerated pressure setting at 1.5 MPa), then each fraction of 0.5 mL was recovered.

The fractions detected as main peaks around 25 to 30 minutes were recovered, and used for analysis. The purified samples were filtrated with the 0.22 μm filter and preserved at 4° C. The protein concentration was calculated from absorbance at 280 nm (OD₂₈₀). As the result, the purified anti-DR3 monovalent antibodies mv142A2 and mv142S38B were obtained.

### Example 9 DR3 Agonism and Antagonism of Anti-DR3 Monoclonal Antibody Fragments

In order to evaluate agonism and antagonism of anti-DR3 monovalent antibodies mv142A2 and mv142S38B established as antibody fragments comprising the Fc region of the antibody in the present invention, the level of phosphorylation of p65 (RelA) NF-κB subunit in human PBMC is analyzed by FCM method same as described in the above Example 6.

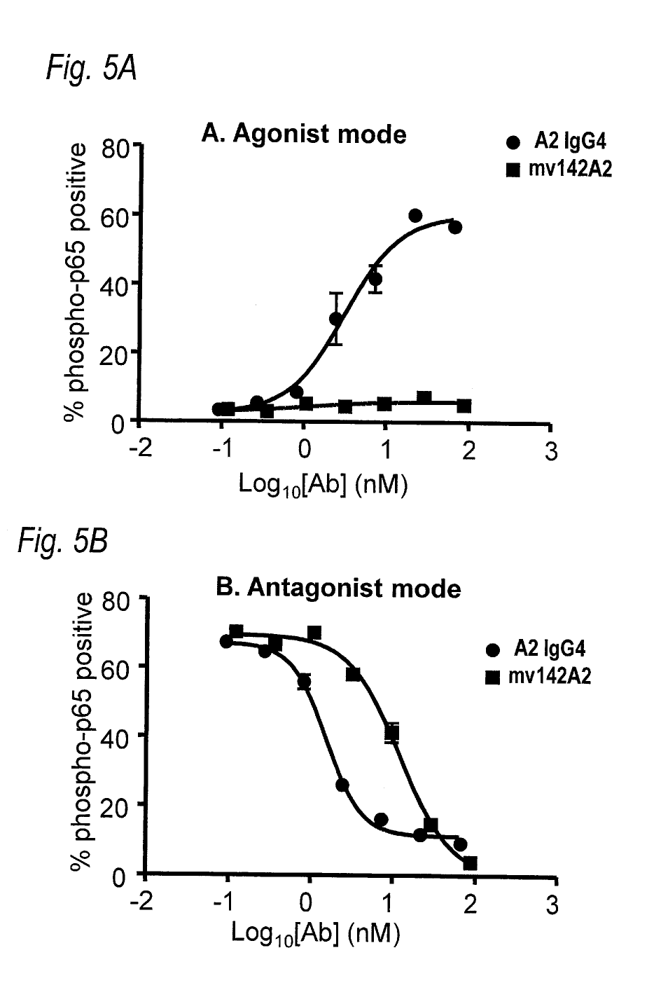

As a result, comparing to TL1A-alone (positive control, and medium-alone (negative control) values were 69.5 and 2.3% respectively in the phosphorylated p65 positive cells in PBMCs, the anti-DR3 monoclonal IgG4 antibody 142A2 IgG4 clearly increased the population of the cell in antibody dose dependent manner, on the other hand, the anti-DR3 monovalent antibody mv142A2 almost never increased the population of the cell in any antibody dose (FIG. 5A).

Thus it was found that the anti-DR3 monovalent antibody mv142A2 clearly showed a lowered or almost no agonistic activity for DR3 compared to anti-DR3 monoclonal antibody 142A2 IgG4. On the other hand, comparing to positive control and negative control, the anti-DR3 monovalent antibody mv142A2 and anti-DR3 monoclonal antibody 142A2 IgG4 decreased the TL1A ligand induced population of phosphorylated p65 positive cells in PBMCs in an antibody dose dependent manner, and the inhibition of mv142A2 was slightly weakened than that of 142A2 IgG4 (FIG. 5B).

Accordingly the anti-DR3 monovalent antibody mv142A2 showed antagonistic activity for TL1A-induced DR3 activation in human PBMC, wherein the antibody has decreased or no agonistic potency for DR3 activity. The antagonistic activity of mv142A2 was slightly decreased than 142A2 IgG4 antibody.

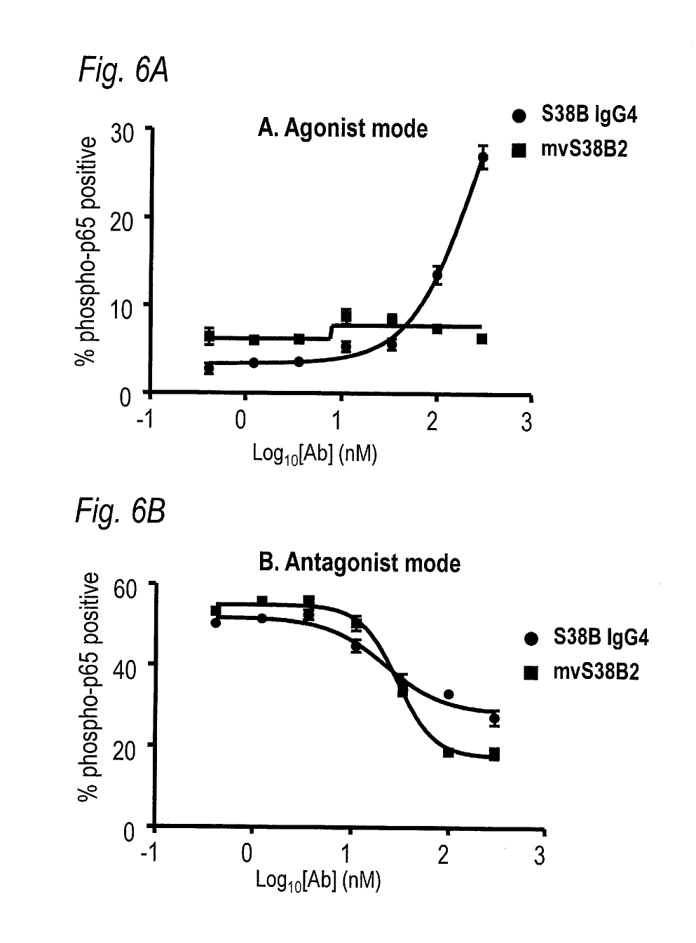

Regarding to the anti-DR3 monovalent antibody mv142S38B, compared to the positive (TL1A alone, 45%) and negative (medium alone, 3%) control values in this experiment, t, the anti-DR3 monoclonal IgG4 antibody 142S38B-IgG4 clearly increased the phosphorylated p65 positive cells in PBMCs in an antibody dose dependent manner, the same as 142A2-IgG4, on the other hand, the anti-DR3 monovalent antibody mv142S38B didn't increase the population of the phosphorylated-p65 positive cells in any antibody dose (FIG. 6A).

On the other hand, the anti-DR3 monovalent antibody mvS38B and the anti-DR3 monoclonal antibody 142S38B IgG4 equivalently decreased the population of the phosphorylated-p65 positive cells in PBMCs, the same as 142A2 antibody clone (FIG. 6B). Thus the anti-DR3 monovalent antibody mv142S38B showed antagonistic activity for TL1A-induced DR3 activation in human PBMC, wherein the antibody has decreased or no agonistic potency.

Therefore, the anti-DR3 monovalent antibody can antagonize TL1A-induced DR3 activation, without significant agonist potency demonstrated Furthermore, the antibody fragments comprising the Fc region of the antibody seem to have a longer half-life and a higher stability in human serum by containing the Fc region of the antibody, compared to previously known anti-DR3 antibody fragment such as mere Fabs. Thus, the antibody fragment including the monovalent antibody composed of H chain and Fc fused L chain of the present invention can be selected as one candidate of pure antagonist for diseases related to aberrant DR3 activation.

### Example 10 Generation of Hinge Region Swapped IgG Variants

In order to determine an essential region derived from an IgG2 antibody in the agonistic activity cancellation, hinge region swapped IgG variants between IgG2 and IgG4 subclasses were generated. Regarding the IgG4 antibody, although Ser residue at position 228 (Eu numbering) in a hinge region has been known to be related to an instability of IgG4 molecule such as half-body, an IgG4 hinge region comprising S228P substitution included in previously described “Nullbody” was used. The IgG variant comprising CH1, CH2 and CH3 domains derived from IgG4 antibody and hinge domain derived from IgG2 antibody was designated as “4244” variant, and the IgG variant comprising CH1, CH2 and CH3 domains derived from IgG2 antibody and hinge domain derived from IgG4 antibody was designated as “2422” variant.

An expression vector was created for production of 142A2-IgG4244 variant heavy chain constant region in which the hinge of IgG4-null antibody was replaced with the hinge of human IgG2. The cDNA encoding for the hinge domain within human “IgG4-null” constant heavy (ESKYGPPCPPCP) was replaced with cDNA encoding for the hinge domain of human IgG2 constant heavy (ERKCCVECPPCP), and the cDNA encoding 142A2-IgG4244 variant was inserted into an appropriate restriction sites on an expression vector, as previously described. As same as the IgG4244 variant, an expression vector including cDNA encoding 142A2-IgG2422 variant was produced. Finally purified each antibody was obtained same as the above procedure described in Examples 1 and 2.

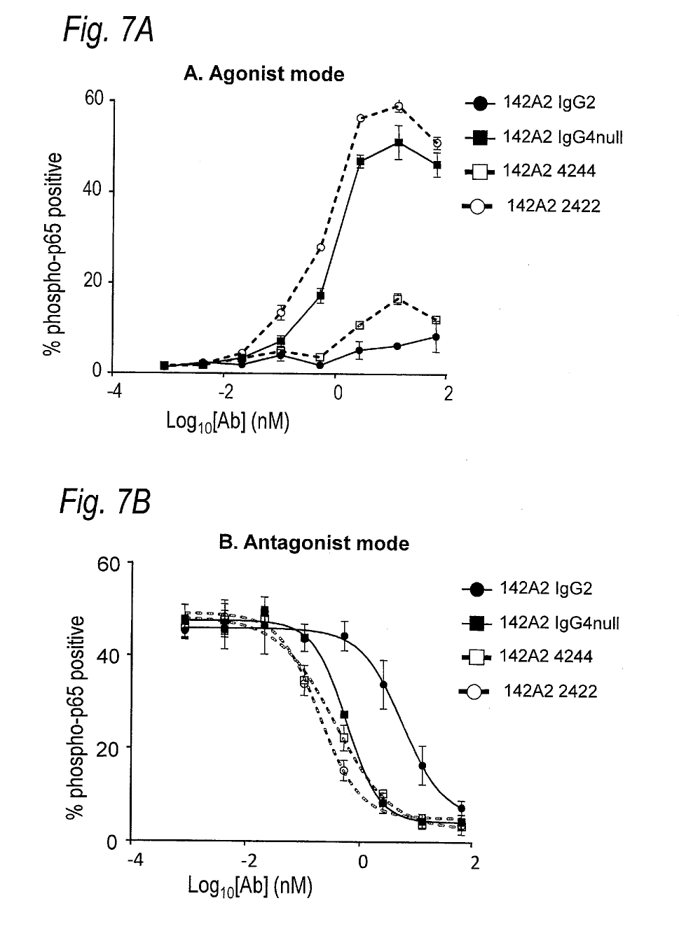

These obtained 142A2 antibody variants were assessed in the same antagonist and agonist assay as described in Example 6. As shown in FIG. 7, while all 142A2 antibodies and their variants displayed robust antagonist activity on p65 phosphorylation induced by TL1A ligand, and IgG4244 and IgG2422 variants especially displayed higher antagonistic activity compared to each parent IgG2 or IgG4-antibody variant (FIG. 7B). On the other hand, although IgG4 and IgG2422 antibody variants displayed significant agonistic activity, IgG2 antibody and IgG4244 antibody variant displayed dramatically reduced agonist activity (FIG. 7A).

These results clearly show that the rearrangement of an IgG constant region to IgG2 subclass reduces agonistic activity of an anti-DR3 antibody 142A2 with no reduction of antagonistic activity. Furthermore, the hinge region of IgG2 class antibody contains essential parts of a rearrangement in order to reduce agonistic activity of an anti-DR3 antibody. Therefore it was found that anti-DR3 antibody which has a hinge region originated from IgG2 subclass is very useful antibody to exhibit a pure antagonistic activity with a significant reduced agonistic activity.

### Example 11 Establishment of 142A2 Antibody Variants

In order to establish antibody variants with an increased affinity, a 142A2 variant single chain Fv (scFv) combinatorial phage-display library was constructed using a standard degenerate oligonucleotide approach. Mutations were preferentially targeted to all 6 CDR regions of the 142A2 antibody. Phage variants with DR3-binding activity were initially identified by panning on immobilized recombinant DR3-Fc fusion protein obtained in the above embodiment.

A dissociation constant (K_{D}) of a screened scFv phage clone were assessed by competitive binding AlphaScreen as described in Proc Natl Acad Sci USA. 2006 Mar. 14; 103(11):4005-4010. Mutants with increased K_{D} over parental 142A2 scFv were subcloned into bacterial expression vectors and scFvs proteins were purified using standard molecular biology techniques. All mutations outside of CDRs in antibody variants were reverted back to parental 142A2 amino acid sequence in order to maintain the amino acid sequence derived from the parent antibody clone, while CDR mutations were retained for further testing. Furthermore, for the VH, Gly at position 112 was substituted with Arg.

As a result, each amino acid sequence of VH and VL such as VH_G112R (SEQ ID NO:76), VL_A51E (SEQ ID NO:77), VL_L54Q (SEQ ID NO:78) and VL_A51E/L54Q (SEQ ID NO:79) are established. Each amino acid sequence of the mutated CDR is depicted as HCDR_G112R (SEQ ID NO:80), LCDR2_A51E (SEQ ID NO:81), LCDR2_L54Q (SEQ ID NO:82) and LCDR2_A51E/L54Q (SEQ ID NO:83) (Table 2).

TABLE 2
amino acid sequences of CDRs in 142A2 antibody variants.

| | | CDR1 | | CDR2 | | CDR3 | |
|---|---|---|---|---|---|---|---|
| | | a.a. # | | a.a. # | | a. a. # | |
| Variation | Domain | range | Sequence | range | Sequence | range | Sequence |
| Parental | VH | 31- | GYSAAWN | 52-69 | RTYYRSKWY | 102- | DYYGSESYYNG |
| | | 37 | | | NDYAVSVKS | 121 | GYYYYGMDV |
| G112R | VH | | GYSAAWN | | RTYYRSKWY | | DYYGSESYYNR |
| | | | | | NDYAVSVKS | | GYYYYGMDV |
| Parental | VL | 24- | RASPGISSA | 50-56 | DASSLES | 89- | QQFNDYPLT |
| | | 34 | LA | | | 97 | |
| A51E | VL | | RASPGISSA | | DESSLES | | QQFNDYPLT |
| | | | LA | | | | |
| L54Q | VL | | RASPGISSA | | DASSQES | | QQFNDYPLT |
| | | | LA | | | | |
| A51E, | VL | | RASPGISSA | | DESSQES | | QQFNDYPLT |
| L54Q | | | LA | | | | |

* Bold and underlined amino acid residues indicate the substituted one. “a.a. # range” indicates the amino acid number of CDR in each variable region.

Recombinant IgG1 versions comprising 142A2 VL_A51E, VL_L54Q and/or VH_G112R, or combinations thereof were constructed, and Fab fragments were generated as described above. The DR3 binding affinity of these 142A2 variants was determined via surface plasmon resonance (SPR) assay same as the above described. All generated 142A2 antibody variants exhibited an improved binding affinity to DR3 protein compared to parental 142A2 (Table 3). The each antibody variant (A2-E, Q, EQ, ER or EQR) comprising A51E, L54Q and/or G112R substitutions in the VH and VL particularly had the highest binding affinity to DR3 protein than the other variants and it had approximately 1.8, 1.2, 1.9, 2.0 or 2.2-fold increase, respectively. Therefore it is very hopeful candidate for DR3 antagonistic therapy.

TABLE 3
142A2 variant DR3-binding affinity
measured by surface plasmon resonance (SPR)

| | | K_{D} (STDEV) |
|---|---|---|
| Anti-DR3 Fab | Kd (nM) | (nM) |
| 142A2 n = 6 | 1.02e−3 | 3.99 (±0.34) |
| 142A2-A51E n = 6 | 8.03e−4 | 2.20 (±.20) |
| 142A2-A51E/L54Q n = 23 | 7.81e−4 | 2.13 (±.13) |
| 142A2-L54Q n = 3 | 9.09e−4 | 3.04 (±.04) |
| 142A2-G112R n = 3 | 1.03e−3 | 3.19 (±.19) |
| 142A2-G112R/A51E n = 6 | 7.45e−4 | 1.96 (±.96) |
| 142A2-G112R/L54Q n = 3 | 8.66e−4 | 2.37 (±.37) |
| 142A2-G112R/A51E/L54Q n = 3 | 7.45e−4 | 1.78 (±.78) |
| 142S38 n = 3 | 3.75e−5 | 0.13 (±.13) |

The number of experimentation was indicated in “n”. “Kd” indicates a dissociation rate constant (nM), and “KD” indicates a dissociation constant (nM).

Furthermore, the 142A2 variant A2-EQR was analyzed in the potency of antagonistic activity. These antagonistic activities of Fab antibody fragments are determined in the same assay in the above experiment. As a result these antibodies inhibited the IL-13 release in dose dependency and completely inhibited TL1A ligand induced IL-13 secretion at 10 nM under presence of anti-CD3 antibody and anti-CD28 antibody combination. Furthermore A2-EQR antibody variant had much higher antagonistic activity compared to the parent A2 antibody. Therefore it was found that anti-DR3 antagonistic antibody variant A2-EQR is very useful for blocking TL1A ligand induced T cell activation.

While the present invention has been described in detail and with reference to specific embodiments thereof, it will be apparent to one skilled in the art that various changes and modifications can be made therein without departing from the spirit and scope thereof. This application is based on U.S. Provisional Application No. 61/975,214 (filed Apr. 4, 2014), and the contents thereof are herein incorporated by reference.

### SEQUENCE LISTING FREE TEXT

SEQ ID NO:1—the nucleotide sequence of TL1A-flag
SEQ ID NO:2—synthetic construct
SEQ ID NO:5—the nucleotide sequence of hDR3-Fc
SEQ ID NO:6—synthetic construct
SEQ ID NO:7—the nucleotide sequence of hDR3-mG1Fc
SEQ ID NO:8—synthetic construct
SEQ ID NO:9—the nucleotide sequence of hDR3-DD
SEQ ID NO:10—synthetic construct
SEQ ID NO:11—the nucleotide sequence of mDR3-DD
SEQ ID NO:12—synthetic construct
SEQ ID NO:13—the nucleotide sequence of 142A2-VH full
SEQ ID NO:14—synthetic construct
SEQ ID NO:15—the amino acid sequence of 142A2-VH
SEQ ID NO:16—the amino acid sequence of 142A2-HCDR1
SEQ ID NO:17—the amino acid sequence of 142A2-HCDR2
SEQ ID NO:18—the amino acid sequence of 142A2-HCDR3
SEQ ID NO:19—the nucleotide sequence of 142A2-VL full
SEQ ID NO:20—synthetic construct
SEQ ID NO:21—the amino acid sequence of 142A2-VL
SEQ ID NO:22—the amino acid sequence of 142A2-LCDR1
SEQ ID NO:23—the amino acid sequence of 142A2-LCDR2
SEQ ID NO:24—the amino acid sequence of 142A2-LCDR3
SEQ ID NO:25—the nucleotide sequence of 142S38B-VH full
SEQ ID NO:26—synthetic construct
SEQ ID NO:27—the amino acid sequence of 142S38B-VH
SEQ ID NO:28—the amino acid sequence of 142S38B-HCDR1
SEQ ID NO:29—the amino acid sequence of 142S38B-HCDR2
SEQ ID NO:30—the amino acid sequence of 142S38B-HCDR3
SEQ ID NO:31—the nucleotide sequence of 142S38B-VL full
SEQ ID NO:32—synthetic construct
SEQ ID NO:33—the amino acid sequence of 142S38B-VL
SEQ ID NO:34—the amino acid sequence of 142S38B-LCDR1
SEQ ID NO:35—the amino acid sequence of 142S38B-LCDR2
SEQ ID NO:36—the amino acid sequence of 142S38B-LCDR3
SEQ ID NO:37—the amino acid sequence of hCL kappa
SEQ ID NO:38—the amino acid sequence of hIgG1_CH
SEQ ID NO:39—the amino acid sequence of hIgG2_CH
SEQ ID NO:40—the amino acid sequence of hIgG4 variant
SEQ ID NO:41—the nucleotide sequence of hTL1A-3′_F
SEQ ID NO:42—the nucleotide sequence of hTL1A-3′_R
SEQ ID NO:43—the nucleotide sequence of VCAM-FLAGR-SalI_F
SEQ ID NO:44—the nucleotide sequence of hTL1A-EC_BamHI-R
SEQ ID NO:45—the nucleotide sequence of VCAM-FLAG_hTL1A_F
SEQ ID NO:46—the nucleotide sequence of VCAM-FLAG_hTL1A_R
SEQ ID NO:47—the nucleotide sequence of hDR3_F5′
SEQ ID NO:48—the nucleotide sequence of hDR3-EC_R
SEQ ID NO:49—the nucleotide sequence of hDR3-SalI_F
SEQ ID NO:50—the nucleotide sequence of hDR3_hG1Fc_R
SEQ ID NO:51—the nucleotide sequence of hDR3_hG1Fc_F
SEQ ID NO:52—the nucleotide sequence of hDR3_hG1Fc_BamHI-R
SEQ ID NO:53—the nucleotide sequence of hDR3-pG-NheI_R
SEQ ID NO:54—the nucleotide sequence of mG1Fc-pG-NheI_F
SEQ ID NO:55—the nucleotide sequence of mG1Fc_BamHI-R
SEQ ID NO:56—the nucleotide sequence of hDR3ΔDD_BamHI_R
SEQ ID NO:57—the nucleotide sequence of mDR3_Fwd
SEQ ID NO:58—the nucleotide sequence of mDR3_3′_R
SEQ ID NO:59—the nucleotide sequence of mDR3-SalI_F
SEQ ID NO:60—the nucleotide sequence of mDR3(1-324)_BamHI
SEQ ID NO:61—the nucleotide sequence of IgG1 primer
SEQ ID NO:62—the nucleotide sequence of hk5 primer
SEQ ID NO:63—the nucleotide sequence of 142S38BA_VLF_Bgll
SEQ ID NO:64—the nucleotide sequence of 142P20_VL1_BsiWI
SEQ ID NO:65—the nucleotide sequence of 142A2_BglI_F
SEQ ID NO:66—the nucleotide sequence of 142A2_BsiWI_R
SEQ ID NO:67—the nucleotide sequence of 142A2-VH_SalI_F
SEQ ID NO:68—the nucleotide sequence of 142A2-VH_NheI_R
SEQ ID NO:69—the nucleotide sequence of 142S38BA_VHF_SalI
SEQ ID NO:70—the nucleotide sequence of F429_VH_R1
SEQ ID NO:71—the nucleotide sequence of IgG2 primer
SEQ ID NO:72—the amino acid sequence of 142A2_mvG4_HC
SEQ ID NO:73—the amino acid sequence of 142A2_mvG4_LC
SEQ ID NO:74—the amino acid sequence of 142S38B_mvG4_HC
SEQ ID NO:75—the amino acid sequence of 142S38B_mvG4_LC
SEQ ID NO:76—the amino acid sequence of 142A2-VH_G112R
SEQ ID NO:77—the amino acid sequence of 142A2-VL_A51E
SEQ ID NO:78—the amino acid sequence of 142A2-VL_L54Q
SEQ ID NO:79—the amino acid sequence of 142A2-VL_A51E/L54Q
SEQ ID NO:80—the amino acid sequence of 142A2-HCDR_G112R
SEQ ID NO:81—the amino acid sequence of 142A2-LCDR2_A51E
SEQ ID NO:82—the amino acid sequence of 142A2-LCDR2_L54Q
SEQ ID NO:83—the amino acid sequence of 142A2-LCDR2_A51E/L54Q

### Sequence Listing

1. An immunoglobulin G (hereinafter described as IgG) antibody which binds to death receptor 3 (DR3) and antagonizes TL1A induced DR3 activation, wherein the antibody has a decreased or no agonistic activity against DR3 through their binding, or an antibody fragment thereof.

2. The antibody or the antibody fragment thereof according to claim 1, which binds to an epitope presented in a cysteine-rich domain (hereinafter described as CRD) of DR3.

3. The antibody and the antibody fragment thereof according to claim 1, which binds to an epitope comprising at least one amino acid residue presented in CRD1 or CRD4 of DR3.

4. The antibody or the antibody fragment thereof according to claim 1, which is one selected from an IgG2 antibody, an IgG2 antibody variant comprising a hinge domain of IgG2, and a domain exchanged antibody between IgG2 and IgG4, wherein an amino acid residue is Lys at EU numbering position 409.

5. The antibody or the antibody fragment thereof according to claim 1, which neutralizes and/or antagonizes an activity of DR3 induced through TL1A ligand binding.

6. The antibody or the antibody fragment thereof according to claim 1, wherein the agonistic activity is at least one selected from the phosphorylation of p65 subunit of NF-kappa B, cytokine release from DR3 expressed cells, the proliferation of DR3 expressed cells, the apoptosis of DR3 expressed cells.

7. The antibody or the antibody fragment thereof according to claim 1, which is at least one antibody selected from (i) to (iii) as described following;
(i) an antibody which competitively binds to DR3 with the anti-DR3 monoclonal antibody 142A2 or 142S38B,
(ii) an antibody which binds to an epitope presented in the epitope recognized by the anti-DR3 monoclonal antibody 142A2 or 142S38B, and
(iii) an antibody which binds to same epitope recognized by the anti-DR3 monoclonal antibody 142A2 or 142S38B.

8. The antibody or the antibody fragment thereof according to claim 1, which comprises an amino acid sequence of VH of SEQ ID NO: 15 and an amino acid sequence of VL of SEQ ID NO:21, an amino acid sequence of VH of SEQ ID NO:76 and an amino acid sequence of VL of SEQ ID NO:21, an amino acid sequence of VH of SEQ ID NO:15 and an amino acid sequence of VL of SEQ ID NO:77, an amino acid sequence of VH of SEQ ID NO: 15 and an amino acid sequence of VL of SEQ ID NO:78, an amino acid sequence of VH of SEQ ID NO:15 and an amino acid sequence of VL of SEQ ID NO:79, an amino acid sequence of VH of SEQ ID NO:76 and an amino acid sequence of VL of SEQ ID NO:79, or an amino acid sequence of VH of SEQ ID NO:27 and an amino acid sequence of VL of SEQ ID NO:33.

9. The antibody or the antibody fragment thereof according to claim 1, which comprises an amino acid sequences of CDR1 to CDR3 of VH of SEQ ID NOs: 16 to 18, respectively, and an amino acid sequences of CDR1 to CDR3 of VL of SEQ ID NOs:22 to 24, respectively; an amino acid sequences of CDR1 to CDR3 of VH of SEQ ID NOs:16, 17, 80, respectively, and an amino acid sequences of CDR1 to CDR3 of VL of SEQ ID NOs:22-24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 81, 24, respectively; an amino acid sequences of CDR1 to CDR3 of VH of SEQ ID NOs: 16, 17, 80, respectively, and an amino acid sequences of CDR1 to CDR3 of VL of SEQ ID NOs:22, 82, 24, respectively; an amino acid sequences of CDR1 to CDR3 of VH of SEQ ID NOs: 16-18, respectively, and an amino acid sequences of CDR1 to CDR3 of VL of SEQ ID NOs:22, 81, 24, respectively; an amino acid sequences of CDR1 to CDR3 of VH of SEQ ID NOs: 16-18, respectively, and an amino acid sequences of CDR1 to CDR3 of VL of SEQ ID NOs:22, 82, 24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 83, 24, respectively; or an amino acid sequences of CDR1 to CDR3 of VH of SEQ ID NOs:28 to 30, respectively, and amino acid sequences of CDR1 to CDR3 of VL of SEQ ID NOs:34 to 36, respectively.

10. The antibody or the antibody fragment according to claim 1, wherein the antibody fragment is selected from Fab, Fab′, F(ab′)2, single chain Fv (scFv), diabody, disulfide stabilized Fv (dsFv), a peptide comprising six CDRs of the antibody and a Fc fusion proteins.

11. The antibody or the antibody fragment thereof according to claim 10, wherein the Fc fusion protein is an Fab or scFv fused to a Fc region selected from as following;
(i) a bivalent antibody in that two Fabs or scFvs are fused to Fc region of IgG class,
(ii) a monovalent antibody in that one Fab or scFv is fused to Fc region, and
(iii) a monovalent antibody comprising a H chain and a Fc-fused L-chain (hereinafter described as FL).

12. The antibody or the antibody fragment thereof according to claim 11, wherein the Fc region is selected from IgG1, IgG2, IgG4 and a variant thereof.

13. The antibody or the antibody fragment hereof according to claim 10, which comprises an amino acid sequence of VH of SEQ ID NO: 15 and an amino acid sequence of VL of SEQ ID NO:21, an amino acid sequence of VH of SEQ ID NO:76 and an amino acid sequence of VL of SEQ ID NO:21, an amino acid sequence of VH of SEQ ID NO:15 and an amino acid sequence of VL of SEQ ID NO:77, an amino acid sequence of VH of SEQ ID NO: 15 and an amino acid sequence of VL of SEQ ID NO:78, an amino acid sequence of VH of SEQ ID NO:15 and an amino acid sequence of VL of SEQ ID NO:79, an amino acid sequence of VH of SEQ ID NO:76 and an amino acid sequence of VL of SEQ ID NO:79, or an amino acid sequence of VH of SEQ ID NO:27 and an amino acid sequence of VL of SEQ ID NO:33.

14. The antibody or the antibody fragment hereof according to claim 10, which comprises an amino acid sequences of CDR1 to CDR3 of VH of SEQ ID NOs:16 to 18, respectively, and amino acid sequences of CDR1 to CDR3 of VL of SEQ ID NOs:22 to 24, respectively; an amino acid sequences of CDR1 to CDR3 of VH of SEQ ID NOs:16, 17, 80, respectively, and an amino acid sequences of CDR1 to CDR3 of VL of SEQ ID NOs:22-24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 81, 24, respectively; an amino acid sequences of CDR1 to CDR3 of VH of SEQ ID NOs: 16, 17, 80, respectively, and an amino acid sequences of CDR1 to CDR3 of VL of SEQ ID NOs:22, 82, 24, respectively; an amino acid sequences of CDR1 to CDR3 of VH of SEQ ID NOs: 16-18, respectively, and an amino acid sequences of CDR1 to CDR3 of VL of SEQ ID NOs:22, 81, 24, respectively; an amino acid sequences of CDR1 to CDR3 of VH of SEQ ID NOs: 16-18, respectively, and an amino acid sequences of CDR1 to CDR3 of VL of SEQ ID NOs:22, 82, 24, respectively; an amino acid sequences of CDR1 to CDR3 of VH of SEQ ID NOs:16, 17, 80, respectively, and an amino acid sequences of CDR1 to CDR3 of VL of SEQ ID NOs:22, 83, 24, respectively; or amino acid sequences of CDR1 to CDR3 of VH of SEQ ID NOs:28 to 30, respectively, and amino acid sequences of CDR1 to CDR3 of VL of SEQ ID NOs:34 to 36, respectively.


SEQUENCE LISTING (83 sequences)

SEQ ID NO: 1 - DNA, 639 bp, Artificial Sequence
  Synthetic construct
atgcctgggaagatggtcgtgatccttggagcctcaaatatactttggataatgtttgcagcttctcaagctgactacaaggacgacgatgacaagctaaaaggacaggagtttgcaccttcacatcagcaagtttatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactatag

SEQ ID NO: 2 - protein, 212 aa, Artificial Sequence
  Synthetic construct
MPGKMVVILGASNILWIMFAASQADYKDDDDKLKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 3 - DNA, 1254 bp, Homo sapiens
  CDS (1)..(1254)
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtga

SEQ ID NO: 4 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 5 - DNA, 1299 bp, Artificial Sequence
  Synthetic construct
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 432 aa, Artificial Sequence
  Synthetic construct
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 936 bp, Artificial Sequence
  Synthetic construct
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgctagcggaggaggatctggaggaggaagtggaggaggatctcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 8 - protein, 311 aa, Artificial Sequence
  Synthetic construct
MEQRPRGCAAVAAALLLVLLGARAQGGTRSPRCDCAGDFHKKIGLFCCRGCPAGHYLKAPCTEPCGNSTCLASGGGSGGGSGGGSPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - DNA, 1038 bp, Artificial Sequence
  Synthetic construct
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggtga

SEQ ID NO: 10 - protein, 345 aa, Artificial Sequence
  Synthetic construct
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPAR

SEQ ID NO: 11 - DNA, 975 bp, Artificial Sequence
  Synthetic construct
atggaggcacggctgctgcggggctgcgtggtggagcctctgttcctaccactgctgctgctgctgctgctgctgcttggtggccagggccagggcggcatgtctggcaggtgtgactgtgccagtgagtcccagaagaggtatggcccgttttgttgcaggggctgcccaaagggacactacatgaaggccccctgcgcagaaccctgtggcaactccacctgccttccctgtccctcggacaccttcttgaccagagacaaccactttaagactgactgtacccgctgccaagtctgtgatgaagaggcccttcaagtgacccttgagaactgctcggcaaagtcggacacccactgtggctgccagtcaggctggtgtgttgactgctccaccgagccatgtgggaaaagctcacctttctcttgtgtcccatgcggggctacaacaccagtccatgaggctccaaccccccggccctgcctgcctggcttctatatacgtggcaatgactgcacgtcctgccccacgggcttcagcagcgtttgccctaaggcttgcactgctgtctgtggctggaagcagatgttttgggtccaggtgcttctaggagtcgcgttcctttttggggctatcctgatctgtgcatattgtcgatggcagccttgtaaggccgtggtcactgcagacacagctgggacggagaccctggcctcaccacagactgcccatctctcagcctcagacagcgcccacaccctcctggcacctccaagcagtactgggaaaatctgtaccactgtccagttggtaggcaacaactggacccctggcttatcccagactcaggaggtggtctgcggacaggcctcacaaccctgggatcagctgccaaacagaactcttggaactcctctggcatctccgctctcgccagcgccccctgcgggctctccggctgctgtgctccagtga

SEQ ID NO: 12 - protein, 324 aa, Artificial Sequence
  Synthetic construct
MEARLLRGCVVEPLFLPLLLLLLLLLGGQGQGGMSGRCDCASESQKRYGPFCCRGCPKGHYMKAPCAEPCGNSTCLPCPSDTFLTRDNHFKTDCTRCQVCDEEALQVTLENCSAKSDTHCGCQSGWCVDCSTEPCGKSSPFSCVPCGATTPVHEAPTPRPCLPGFYIRGNDCTSCPTGFSSVCPKACTAVCGWKQMFWVQVLLGVAFLFGAILICAYCRWQPCKAVVTADTAGTETLASPQTAHLSASDSAHTLLAPPSSTGKICTTVQLVGNNWTPGLSQTQEVVCGQASQPWDQLPNRTLGTPLASPLSPAPPAGSPAAVLQ

SEQ ID NO: 13 - DNA, 456 bp, Artificial Sequence
  Synthetic construct
atgtctgtctccttcctcatcttcctgcccgtgctgggcctcccatggggtgtcctgtcacaggtacagctacagcagtcaggtccaggactggtgaagccctcgcagaccctctcactcacctgtgccatctccggggacagtgtctctgggtacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagattactatggttcggagagttattataacggggggtactactactacggtatggacgtctggggccaagggaccacggtcaccgtctcctca

SEQ ID NO: 14 - protein, 152 aa, Artificial Sequence
  Synthetic construct
MSVSFLIFLPVLGLPWGVLSQVQLQQSGPGLVKPSQTLSLTCAISGDSVSGYSAAWNWIRQSPSRGLEWLGRTYYRSKWYNDYAVSVKSRITINPDTSKNQFSLQLNSVTPEDTAVYYCARDYYGSESYYNGGYYYYGMDVWGQGTTVTVSS

SEQ ID NO: 15 - protein, 132 aa, Artificial Sequence
  Synthetic construct
QVQLQQSGPGLVKPSQTLSLTCAISGDSVSGYSAAWNWIRQSPSRGLEWLGRTYYRSKWYNDYAVSVKSRITINPDTSKNQFSLQLNSVTPEDTAVYYCARDYYGSESYYNGGYYYYGMDVWGQGTTVTVSS

SEQ ID NO: 16 - protein, 7 aa, Artificial Sequence
  Synthetic construct
GYSAAWN

SEQ ID NO: 17 - protein, 18 aa, Artificial Sequence
  Synthetic construct
RTYYRSKWYNDYAVSVKS

SEQ ID NO: 18 - protein, 20 aa, Artificial Sequence
  Synthetic construct
DYYGSESYYNGGYYYYGMDV

SEQ ID NO: 19 - DNA, 387 bp, Artificial Sequence
  Synthetic construct
atggacatgagggtccccgctcagctcctggggcttctgctgctctggctcccaggtgccagatgtgccatccagttgacccagtctccatcctccctgtctgcatctgtaggagacagagtcaccatcacttgccgggcaagtccgggcattagcagtgctttagcctggtatcagcagaaaccagggaaagctcctaagctcctgatgtatgatgcctccagtttggaaagtggggtcccatcaaggttcagcggcagtggatctgggacagatttcactctcaccatcagcagcctgcagcctgaagattttgcaacttattactgtcaacagtttaatgattacccgctcactttcggcggagggaccaaggtggagatcaaa

SEQ ID NO: 20 - protein, 129 aa, Artificial Sequence
  Synthetic construct
MDMRVPAQLLGLLLLWLPGARCAIQLTQSPSSLSASVGDRVTITCRASPGISSALAWYQQKPGKAPKLLMYDASSLESGVPSRFSGSGSGTDFTLTISSLQPEDFATYYCQQFNDYPLTFGGGTKVEIK

SEQ ID NO: 21 - protein, 107 aa, Artificial Sequence
  Synthetic construct
AIQLTQSPSSLSASVGDRVTITCRASPGISSALAWYQQKPGKAPKLLMYDASSLESGVPSRFSGSGSGTDFTLTISSLQPEDFATYYCQQFNDYPLTFGGGTKVEIK

SEQ ID NO: 22 - protein, 11 aa, Artificial Sequence
  Synthetic construct
RASPGISSALA

SEQ ID NO: 23 - protein, 7 aa, Artificial Sequence
  Synthetic construct
DASSLES

SEQ ID NO: 24 - protein, 9 aa, Artificial Sequence
  Synthetic construct
QQFNDYPLT

SEQ ID NO: 25 - DNA, 408 bp, Artificial Sequence
  Synthetic construct
atggagtttgggctgagctggctttttcttgtggctattttaaaaggtgtccagtgtgaggtgcagctgttggagtctgggggaggcttggtacagcctggggggtccctgagactctcctgtgcagcctctggattcaccgttagcagatatgccatgagctgggtccgccaggctccagggaaggggctggagtgggtctcagctattagtgatagtggtggtcgcacatactacgcagactccgtgatgggccggttcaccatctccagagacacttccaagaacacgctgtatctccaaatgaacagcctgagagccgaggacacggccgtatatttctgtgcgaaagagttcggtgactaccttgactactggggccagggaaccctggtcaccgtctcctca

SEQ ID NO: 26 - protein, 136 aa, Artificial Sequence
  Synthetic construct
MEFGLSWLFLVAILKGVQCEVQLLESGGGLVQPGGSLRLSCAASGFTVSRYAMSWVRQAPGKGLEWVSAISDSGGRTYYADSVMGRFTISRDTSKNTLYLQMNSLRAEDTAVYFCAKEFGDYLDYWGQGTLVTVSS

SEQ ID NO: 27 - protein, 117 aa, Artificial Sequence
  Synthetic construct
EVQLLESGGGLVQPGGSLRLSCAASGFTVSRYAMSWVRQAPGKGLEWVSAISDSGGRTYYADSVMGRFTISRDTSKNTLYLQMNSLRAEDTAVYFCAKEFGDYLDYWGQGTLVTVSS

SEQ ID NO: 28 - protein, 5 aa, Artificial Sequence
  Synthetic construct
RYAMS

SEQ ID NO: 29 - protein, 17 aa, Artificial Sequence
  Synthetic construct
AISDSGGRTYYADSVMG

SEQ ID NO: 30 - protein, 8 aa, Artificial Sequence
  Synthetic construct
EFGDYLDY

SEQ ID NO: 31 - DNA, 378 bp, Artificial Sequence
  Synthetic construct
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggtggacgttcggccaagggaccaaggtggaaatcaaa

SEQ ID NO: 32 - protein, 126 aa, Artificial Sequence
  Synthetic construct
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWWTFGQGTKVEIK

SEQ ID NO: 33 - protein, 106 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWWTFGQGTKVEIK

SEQ ID NO: 34 - protein, 11 aa, Artificial Sequence
  Synthetic construct
RASQSVSSYLA

SEQ ID NO: 35 - protein, 7 aa, Artificial Sequence
  Synthetic construct
DASNRAT

SEQ ID NO: 36 - protein, 8 aa, Artificial Sequence
  Synthetic construct
QQRSNWWT

SEQ ID NO: 37 - protein, 107 aa, Artificial Sequence [sequence omitted]

SEQ ID NO: 38 - protein, 330 aa, Artificial Sequence [sequence omitted]

SEQ ID NO: 39 - protein, 326 aa, Artificial Sequence
  Synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 327 aa, Artificial Sequence
  Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 41 - DNA, 28 bp, Artificial Sequence
  Synthetic construct
atggccgaggatctgggactgagctttg

SEQ ID NO: 42 - DNA, 27 bp, Artificial Sequence
  Synthetic construct
ccgttgtccctgtggaatgccccctac

SEQ ID NO: 43 - DNA, 41 bp, Artificial Sequence
  Synthetic construct
agagaggtcgaccaccatgcctgggaagatggtcgtgatcc

SEQ ID NO: 44 - DNA, 45 bp, Artificial Sequence
  Synthetic construct
agagagggatccctatagtaagaaggctccaaagaaggttttatc

SEQ ID NO: 45 - DNA, 41 bp, Artificial Sequence
  Synthetic construct
cgacgatgacaagctaaaaggacaggagtttgcaccttcac

SEQ ID NO: 46 - DNA, 43 bp, Artificial Sequence
  Synthetic construct
cctgtccttttagcttgtcatcgtcgtccttgtagtcagcttg

SEQ ID NO: 47 - DNA, 23 bp, Artificial Sequence
  Synthetic construct
gaaggcggaaccacgacgggcag

SEQ ID NO: 48 - DNA, 23 bp, Artificial Sequence
  Synthetic construct
tggtggggtcagagcctccatcc

SEQ ID NO: 49 - DNA, 40 bp, Artificial Sequence
  Synthetic construct
agagaggtcgaccaccatggagcagcggccgcggggctgc

SEQ ID NO: 50 - DNA, 40 bp, Artificial Sequence
  Synthetic construct
cacaagatttggggaacatctgcctccagccacagacagc

SEQ ID NO: 51 - DNA, 43 bp, Artificial Sequence
  Synthetic construct
gaggcagatgttccccaaatcttgtgacaaaactcacacatgc

SEQ ID NO: 52 - DNA, 40 bp, Artificial Sequence
  Synthetic construct
agagagggatcctcatttacccggagacagggagaggctc

SEQ ID NO: 53 - DNA, 42 bp, Artificial Sequence
  Synthetic construct
ctccagatcctcctccgctagcaaggcaggtggagttgccgc

SEQ ID NO: 54 - DNA, 64 bp, Artificial Sequence
  Synthetic construct
gctagcggaggaggatctggaggaggaagtggaggaggatctcccagggattgtggttgtaagc

SEQ ID NO: 55 - DNA, 43 bp, Artificial Sequence
  Synthetic construct
agagagggatcctcatttaccaggagagtgggagaggctcttc

SEQ ID NO: 56 - DNA, 39 bp, Artificial Sequence
  Synthetic construct
agagagggatcctcaccgcgctgggaccgcgtccatcac

SEQ ID NO: 57 - DNA, 26 bp, Artificial Sequence
  Synthetic construct
atggaggcacggctgctgcggggctg

SEQ ID NO: 58 - DNA, 30 bp, Artificial Sequence
  Synthetic construct
ctgctacgcaggaccacggcagggggttag

SEQ ID NO: 59 - DNA, 42 bp, Artificial Sequence
  Synthetic construct
agagaggtcgaccaccatggaggcacggctgctgcggggctg

SEQ ID NO: 60 - DNA, 38 bp, Artificial Sequence
  Synthetic construct
agagagggatcctcactggagcacagcagccggagagc

SEQ ID NO: 61 - DNA, 31 bp, Artificial Sequence
  Synthetic construct
tcttgtccaccttggtgttgctgggcttgtg

SEQ ID NO: 62 - DNA, 30 bp, Artificial Sequence
  Synthetic construct
aggcacacaacagaggcagttccagatttc

SEQ ID NO: 63 - DNA, 33 bp, Artificial Sequence
  Synthetic construct
atatagatctccaccatggaagccccagctcag

SEQ ID NO: 64 - DNA, 32 bp, Artificial Sequence
  Synthetic construct
tatacgtacgtttgatttccaccttggtccct

SEQ ID NO: 65 - DNA, 36 bp, Artificial Sequence
  Synthetic construct
atatagatctccaccatgagggtccccgctcagctc

SEQ ID NO: 66 - DNA, 36 bp, Artificial Sequence
  Synthetic construct
atatcgtacgtttgatctccaccttggtccctccgc

SEQ ID NO: 67 - DNA, 40 bp, Artificial Sequence
  Synthetic construct
atatgtcgacccaccatgtctgtctccttcctcatcttcc

SEQ ID NO: 68 - DNA, 30 bp, Artificial Sequence
  Synthetic construct
atatgctagctgaggagacggtgaccgtgg

SEQ ID NO: 69 - DNA, 32 bp, Artificial Sequence
  Synthetic construct
tatagtcgaccaccatggagtttgggctgagc

SEQ ID NO: 70 - DNA, 24 bp, Artificial Sequence
  Synthetic construct
ttgctagctgaggagacggtgacc

SEQ ID NO: 71 - DNA, 26 bp, Artificial Sequence
  Synthetic construct
gtgcacgccgctggtcagggcgcctg

SEQ ID NO: 72 - protein, 459 aa, Artificial Sequence
  Synthetic construct
QVQLQQSGPGLVKPSQTLSLTCAISGDSVSGYSAAWNWIRQSPSRGLEWLGRTYYRSKWYNDYAVSVKSRITINPDTSKNQFSLQLNSVTPEDTAVYYCARDYYGSESYYNGGYYYYGMDVWGQGTTVTVSSASTKGPSVFPLAPSSKSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 73 - protein, 443 aa, Artificial Sequence
  Synthetic construct
AIQLTQSPSSLSASVGDRVTITCRASPGISSALAWYQQKPGKAPKLLMYDASSLESGVPSRFSGSGSGTDFTLTISSLQPEDFATYYCQQFNDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRFTQKSLSLSLGK

SEQ ID NO: 74 - protein, 444 aa, Artificial Sequence
  Synthetic construct
EVQLLESGGGLVQPGGSLRLSCAASGFTVSRYAMSWVRQAPGKGLEWVSAISDSGGRTYYADSVMGRFTISRDTSKNTLYLQMNSLRAEDTAVYFCAKEFGDYLDYWGQGTLVTVSSASTKGPSVFPLAPSSKSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 75 - protein, 442 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRFTQKSLSLSLGK

SEQ ID NO: 76 - protein, 132 aa, Artificial Sequence
  Synthetic construct
QVQLQQSGPGLVKPSQTLSLTCAISGDSVSGYSAAWNWIRQSPSRGLEWLGRTYYRSKWYNDYAVSVKSRITINPDTSKNQFSLQLNSVTPEDTAVYYCARDYYGSESYYNRGYYYYGMDVWGQGTTVTVSS

SEQ ID NO: 77 - protein, 107 aa, Artificial Sequence
  Synthetic construct
AIQLTQSPSSLSASVGDRVTITCRASPGISSALAWYQQKPGKAPKLLMYDESSLESGVPSRFSGSGSGTDFTLTISSLQPEDFATYYCQQFNDYPLTFGGGTKVEIK

SEQ ID NO: 78 - protein, 107 aa, Artificial Sequence
  Synthetic construct
AIQLTQSPSSLSASVGDRVTITCRASPGISSALAWYQQKPGKAPKLLMYDASSQESGVPSRFSGSGSGTDFTLTISSLQPEDFATYYCQQFNDYPLTFGGGTKVEIK

SEQ ID NO: 79 - protein, 107 aa, Artificial Sequence
  Synthetic construct
AIQLTQSPSSLSASVGDRVTITCRASPGISSALAWYQQKPGKAPKLLMYDESSQESGVPSRFSGSGSGTDFTLTISSLQPEDFATYYCQQFNDYPLTFGGGTKVEIK

SEQ ID NO: 80 - protein, 20 aa, Artificial Sequence
  Synthetic construct
DYYGSESYYNRGYYYYGMDV

SEQ ID NO: 81 - protein, 7 aa, Artificial Sequence
  Synthetic construct
DESSLES

SEQ ID NO: 82 - protein, 7 aa, Artificial Sequence
  Synthetic construct
DASSQES

SEQ ID NO: 83 - protein, 7 aa, Artificial Sequence
  Synthetic construct
DESSQES